(19) United States
(12) Patent Application Publication
(10) Pub. No.: US 2010/0093624 A1
(43) Pub. Date: Apr. 15, 2010

(54) PEPTIDE THERAPEUTICS THAT BIND VEGF AND METHODS OF USE THEREOF

(75) Inventors: Don LOW, San Mateo, CA (US); Andreas Jungbluth, Neunkirchen (DE); Gregor Schuermann, Hannover (DE); Michael Mersmann, Braunschweig (DE); Tamas Blandl, San Carlos, CA (US); Katherine E. Hoover, Fremont, CA (US); Eberhard Schneider, Denkte (DE); Ying Hu, Mountain View, CA (US); Peter Wagner, Braunschweig (DE)

Correspondence Address: LAHIVE & COCKFIELD, LLP;FLOOR 30, SUITE 3000, ONE POST OFFICE SQUARE, BOSTON, MA, 02109, US

(73) Assignee: Cosmix Therapeutics LLC, Marborough, MA (US)

(21) Appl. No.: 12/512,892

(22) Filed: Jul. 30, 2009

Related U.S. Application Data
(60) Provisional application No. 61/084,901, filed on Jul. 30, 2008.

(51) Int. Cl. A61K 38/16; C07K 5/10; C07K 7/06; C07K 7/08; C07K 14/00; A61K 38/07; A61K 38/08; A61K 38/10; A61P 9/00; G01N 33/53

(52) U.S. Cl. 514/12; 530/330; 530/329; 530/328; 530/327; 530/326; 530/325; 530/324; 514/18; 514/17; 514/16; 514/15; 514/14; 514/13; 530/317; 436/501

(57) ABSTRACT

The present invention provides peptides and mimetics thereof that bind to VEGF. In preferred embodiments, the peptides of the invention are D type optical isomers which can bind VEGF and which can inhibit or reduce VEGF biological activity.

### RELATED APPLICATIONS

This application claims priority to U.S. Provisional Patent Application No. 61/084,901, filed on Jul. 30, 2008. The entire contents of the foregoing application is incorporated herein by this reference.

### BACKGROUND OF THE INVENTION

Angiogenesis, the growth of new capillary blood vessels, is one of the most pervasive and essential biological events (Jern (2001) Scrip Magazine; N. Wrighton et al. (1996) Science 273, p. 458-464; Edelson and Haan (2002) BioCentury 10, A1-A3; Osborne (2002) BioWorld Today 13, p. 1-3). A number of physiological and pathological processes, such as embryonic development, the formation of inflammatory granulation tissue during wound healing and the growth of malignant solid tumors, are strictly dependent upon the formation of new capillaries. Normally in the adult mammalian organism, physiological angiogenesis occurs infrequently, yet can be rapidly induced in response to diverse physiologic stimuli. One of the most extensively studied of these angiogenesis-dependent physiological processes is normal wound repair (Leibovich and Weisman. (1998) Prog Clin Biol. Res 266, 131-145).

An important feature of wound-associated angiogenesis is that it is locally transient and tightly controlled. The rate of capillary endothelial cell turnover in adult organisms is typically measured in months or years (Engerman et al. (1967) Lab Invest. 17, 738-743; Tannock et al. (1972) Cancer Res. 32, 77-82). However, when normally quiescent endothelial cells lining venules are stimulated, they will degrade their basement membrane and proximal extracellular matrix, migrate directionally, divide and organize into new functioning capillaries invested by a new basal lamina all within a matter of days. This dramatic amplification of the microvasculature is nevertheless temporary, because they virtually disappear as rapidly as they are formed, quickly returning the tissue vasculature to homeostasis. This demonstrates two key aspects of the angiogenic response: first, the formation of new capillary blood vessels is rapid and controlled; and second, it is transient, and characterized by regression to a physiologic steady-state level. The abrupt termination of angiogenesis that accompanies the resolution of the wound response suggests two possible mechanisms of control.

First, under circumstances not well understood, there is probably a marked reduction in the synthesis and/or elaboration of angiogenic mediators. Second, a simultaneous increase occurs in the level of substances that inhibit new vessel growth (Booth et al. (2001) J Natl Cancer Inst Monogr. 29, 16-20). While angiogenesis under conditions of normal wound repair appears to be under strict control and is self-limited, during neoplastic transformation, neovascularization is exaggerated. It appears that tumors are continually renewing and altering their vascular supply (Mallik, et al. (2002) The McKinsey Quarterly 2002).

Interestingly, normal vascular mass is approximately 20% of the total tissue mass, whereas, during tumorigenesis, tumor vascular mass may be as great as 50% of the total tumor (Mallik, et al. (2002) The McKinsey Quarterly 2002). These findings are consistent with the observations that neovascularization is both a marker of preneoplastic lesions as well as an event that perpetuates tumor growth (Mallik, et al. (2002) The McKinsey Quarterly 2002; Folkman et al. (1989) Nature 339, 58-61; Mairorana et al. (1978) Cancer Res. 38, 4409-4414). This is further exemplified by the fact that the magnitude of tumor-derived angiogenesis has been shown to correlate with metastasis of melanoma, prostate cancer, breast cancer, and non-small cell lung cancer (20-23). In addition, these studies would support the notion that tumor-associated angiogenesis is dysregulated, with a biological imbalance that favors the over-exuberant production of local angiogenic factors or the suppression of endogenous angiostatic factors (Mallik, et al. (2002) The McKinsey Quarterly 2002; Folkman et al. (1989) Nature 339, 58-61; Eisenstein et al. (1975) Am. J. Pathol. 81, 337-347).

Although most investigations studying angiogenesis have focused on the identification and mechanism of action of angiogenic factors, recent evidence suggests that factors that block angiogenesis may play an equally important role in the control of neovascularization (Eisenstein et al. (1975) Am. J Pathol. 81, 337-347; Sorgente et al. (1975) Lab Invest 32, 217-222; Brem et al. (1975) J Exp Med 141, 427-439; Lee et al. (1983) Science 221, 1185-1187; Langer et al. (1980) Proc Natl Acad Sci USA 77, 4331-4335; Brem et al. (1977) Am J Opthalmol 84, 323-328). These latter studies provide a lead for the development of potentially novel therapeutic approaches to the treatment of vascularized tumors.

Disruption of tumor vascularization by blockade of angiogenesis has been predicted to be a route to selective cancer chemotherapy for decades. In principle, blockade of tumor angiogenesis should result in loss of oxygen and nutrient supply, thereby inhibiting tumor growth. After extensive study by numerous groups, however, only recently have anti-angiogenic therapies begun to show promise in the clinic. Thus far, treatments based on the anti-angiogenesis strategy appear to be relatively free from side effects.

VEGF-1 is a potent pro-angiogenic cytokine which is involved in several normal and disease conditions. Binding of VEGF-1 to its cellular receptor initiates a signal transduction cascade that is critical to diverse processes such as wound healing, ovulation, tumor growth, and rheumatoid arthritis. A number of avenues to inhibition of VEGF signaling are being pursued, including decoy receptors (VEGF-Trap, Regeneron), modified nucleic acid aptamers (Macugen, Eyetech/Pfizer) antibodies directed against VEGF receptors, antisense induced downregulation of VEGF and its receptors and antiangiogenic ribozymes (Angiozyme, Ribozyme Pharmaceuticals) but the best characterized system remains direct blockade of VEGF signaling by monoclonal antibody (Avastin, Genentech).

There is a clear need to develop new anti-VEGF therapeutics which have lower manufacturing costs, better tissue penetration, and improved formulation stability than existing antibody based drugs. Such new approaches offer the promise of more effectively treating a variety of disease conditions.

Here, mirror image library screening is used to identify D-peptides that bind specifically to the highly pro-angiogenic cytokine VEGF and select D-peptides that can block cytokine-mediated signaling for development as therapeutics for AMD, oncological indications, and other disease conditions.

### BRIEF DESCRIPTION OF THE FIGURES

FIG. 1 depicts a comparison of pp27 library binding to 100 nM D-VEGF (white, background bars in 3D plot) vs. biotin loaded streptavidin beads (darker, foreground bars in 3D plot) for each round of selection. mRNA-peptide fusion binding was quantitated by scintillation counting.

FIG. 2 depicts a comparison of pp27 library binding to 1 nM D-VEGF (bars in the back of the 3D plot) vs. biotin loaded streptavidin beads (foreground bars in the 3D plot) for each round of selection. mRNA-peptide fusion binding was quantitated by scintillation counting.

FIG. 3 depicts results of D-peptide therapeutics (DRx) VEGF ELISA data. D-peptide G2226 (topmost line) binds tightly to VEGF165, as does anti-VEGF monoclonal antibody BAF293. In contrast, negative control peptide G2264 shows virtually no binding, as does D-peptide G2235.

FIG. 4 depicts the silver-stained SDS-PAGE gel from bead-binding assay demonstrating stereoselective binding of G2226. Lane 3, beads+G2226+D-VEGF; lane 4, G2226+L-VEGF; lane 5, beads+G2264+D-VEGF; lane 7, D-VEGF; lane 8, L-VEGF. A band corresponding to VEGF appears in 4, but not lane 3, indicating that D-peptide G2226 binds only to L-VEGF.

FIG. 5 depicts a plot of BIACORE resonance intensity vs. VEGF concentration. Dissociation constants ranging from 7-18 nM can be derived from fits to this data (black line).

FIG. 6 depicts the inhibition of VEGF binding to VEGF receptors (KDR, bars on the left; Flt-1 patterned bars in the center, i.e., the three bars starting with the fifth bar from the left) by peptide G2257.

FIG. 7 depicts results of the HUVEC cell-based assay. Peptide 07-D60 appears to inhibit HUVEC growth in a dose dependent manner. A 1:20 dilution corresponds to 10 micromolar peptide concentration.

FIG. 8 is a schematic representation of the linear peptide library p27a1. Open reading frame of the library is pictured as a boxed coding sequence. Translated protein sequence (SEQ ID NO: 48) is shown in single letter amino acid code, where X represents any amino acid encoded by NNS codon on DNA level. Parts of the library that are not translated are indicated as: (a) T7-promoter for efficient in vitro transcription, (b) TMV—Tobacco Mosaic Virus translation initiation sequence to allow efficient in vitro translation.

FIG. 9 is a schematic overview of the various method steps during mRNA display selection technology.

FIG. 10 depicts enrichment of D-VEGF binders during increasing rounds of mRNA display selection. The binding of radioactive labeled peptide-RNA-cDNA-fusions after washing was calculated relative to the respective activity of peptide-RNA-cDNA-fusions used as input at every round of selection. Biotinylated D-VEGF immobilized on magnetic Streptavidin beads was used as target material during the various rounds of selection (net binding to biotinylated D-VEGF); As a control binding of peptide-RNA-cDNA fusions to target free beads were as well analyzed in every round of the selection

FIG. 11 depicts enriched binder sequences from the final round of the mRNA display selection on bead immobilized D-VEGF. After PCR-amplification, ligation into plasmid pSTBlue-1 and cloning in E. coli the encoding cDNAs of enriched binder pools after selection round 4 was subjected to sequence analysis. The corresponding amino acid sequences are shown in one letter code. Variations are bolded and underlined. FIG. 11 discloses SEQ ID NOS 20, 22, 49, 22, 22, 22, 50, 51, 51, 51, 24, 24, 52, 23, 23, 53-55, 55-59, 59-67, 53, 17, 15, 17, 22, 68, 24, 23, 15, 22, 20, 69, 21, 70, 23, 70-72, 17, 23, 24, 73, 24, 74, 75, 55, 21 and 21, respectively, in order of appearance.

FIG. 12 depicts enrichment of D-VEGF binders during increasing rounds of mRNA display selection. The binding of radioactive labeled peptide-RNA-cDNA-fusions after washing was calculated relative to the respective activity of peptide-RNA-cDNA-fusions used as input at every round of selection. Biotinylated D-VEGF immobilized on magnetic Streptavidin beads was used as target material during the various rounds of selection (net binding to biotinylated D-VEGF in white); As a control binding of peptide-RNA-cDNA fusions to target free beads were as well analyzed in every round of the selection

FIG. 13 depicts enriched binder sequences from the final round of the back up mRNA display selection on bead immobilized D-VEGF. After PCR-amplification, ligation into plasmid pSTBlue-1 and cloning in E. coli the encoding cDNAs of enriched binder pools after selection round 4 was subjected to sequence analysis. The corresponding amino acid sequences (SEQ ID NOS 11 and 76-128, respectively, in order of appearance) are shown in one letter code. Variations are bolded and underlined.

FIG. 14 depicts the frequency of mutation occurrence within peptide-RNA-cDNA fusions after back up selection on D-VEGF. The frequency of occurrence of the corresponding Amino acid changes within the sequences are listed. Analysis has been based on a total number of 53 clones. Hot spot mutations found within the first selection are underlined. FIG. 14 discloses SEQ ID NO: 129.

FIG. 15 depicts an ELISA analysis of peptides deriving from sequences of the affinity maturation mRNA display selection at 25 nM. ELISA plates were coated for 60 minutes at 37° C. with human IgG 280 ng/well in PBS, L-VEGF 20 pmol/well in PBS, D-VEGF 20 pmol/well in PBS, 2% milk in PBS respectively and plates were consecutively blocked with 2% Milk in HBS for 30 minutes at RT. Then the plates were incubated with 25 nM of peptides 07-007, 07-071 and 07-072 respectively in HBS buffer for one hour at room temperature followed by 4× washes with HBS buffer. Detection with Streptavidin-Peroxidase stained with o-Phenylendiamin and H₂O₂ for 3 minutes.

FIG. 16 depicts an ELISA analysis of peptides deriving from sequences of the affinity maturation mRNA display selection at 5 μM and 100 nM. ELISA plates were coated for 60 minutes at 37° C. with D-VEGF 20 pmol/well in PBS, 1% BSA in PBS respectively and plates were consecutively blocked with 2% Milk in HBS for 30 minutes at RT. Then the plates were incubated with either 5 μM or 100 nM of peptides 07-007, 07-071 and 07-072 respectively in HBS buffer for one hour at room temperature followed by 4× washes with HBS buffer. Detection with Streptavidin-Peroxidase stained with o-Phenylendiamin and H₂O₂ for 3 minutes.

FIG. 17 depicts an ELISA analysis of peptides deriving from sequences of the affinity maturation mRNA display selection at 5 nM and 50 nM. ELISA plates were coated for 60 minutes at 37° C. with D-VEGF 20 pmol/well in PBS and plates were consecutively blocked with 2% Milk in HBS for 30 minutes at RT. Then the plates were incubated either with 50 nM or 5 nM of peptides 07-007, 07-071 and 07-072 respectively in HBS buffer for one hour at room temperature followed by 4× washes with HBS buffer. Detection with Streptavidin-Peroxidase stained with o-Phenylendiamin and H₂O₂ for 3 minutes.

### SUMMARY OF THE INVENTION

The present invention provides peptides, particularly D-peptides, which bind to VEGF-1. In preferred embodiments the present invention is intended to encompass all D-peptides which can bind VEGF and, in particular, those which inhibit or reduce VEGF biological activity. Accordingly, in one aspect of the invention isolated peptides or mimetics thereof which specifically bind to VEGF are provided, wherein said peptides or mimetics thereof are between 4 and 90 amino acids in length and wherein the amino acids are D type optical isomers.

In one embodiment an isolated peptide or mimetic thereof is provided, wherein the peptide comprises the following formula (SEQ ID NO: 1):

Φ₁-Z₁-G-X₁-X₂-L-X₃-X₄-V-C-X₅-X₆-X₇-X₈-C-W-X₉-X₁₀-X₁₁-W-A-X₁₂-X₁₃-X₁₄-X₁₅-X₁₆-X₁₇-X₁₈-X₁₉-L-Z₂-Φ₂

wherein X₁ is chosen from the group consisting of the amino acids N, Y, F, D, I, and H, X₂ is chosen from the group consisting of the amino acids A, T, and V, X₃ is chosen from the group consisting of the amino acids H, Q, and R, X₄ is chosen from the group consisting of the amino acids W and R, X₅ is chosen from the group consisting of the amino acids A and V, X₆ is chosen from the group consisting of the amino acids S and L, X₇ is chosen from the group consisting of the amino acids N, S, and D, X₈ is chosen from the group consisting of the amino acids I, V, and H, X₉ is chosen from the group consisting of the amino acids R and M, X₁₀ is chosen from the group consisting of the amino acids S, T, P, and F, X₁₁ is chosen from the group consisting of the amino acids P and L, X₁₂ is chosen from the group consisting of the amino acids G, E, R, A, and V, X₁₃ is chosen from the group consisting of the amino acids R, and Q, X₁₄ is chosen from the group consisting of the amino acids L and W, X₁₅ is chosen from the group consisting of the amino acids W and R, X₁₆ is chosen from the group consisting of the amino acids G, R, E, A, V, and W, X₁₇ is chosen from the group consisting of the amino acids L, F, M, W, and Y, X₁₈ is chosen from the group consisting of the amino acids V and I, X₁₉ is chosen from the group consisting of the amino acids R, L, Q, and H, and wherein the formula may encompass conservative amino acid modifications at any position. In such an embodiment 1 to 10 amino acids are optionally inserted or deleted. In some embodiments Z₁ and Z₂ may be each independently absent or may each independently be a peptide of length 1 to 25 composed of any amino acids. In some preferred embodiments Z₁ and/or Z₂ comprise or consist of T, G, GSGS (SEQ ID NO: 2), SGSSSGSGS (SEQ ID NO: 3), TSGGSSGSS (SEQ ID NO: 4), TSGGSSGSSLGVASAI (SEQ ID NO: 5), MHHHHHHSGSSSGSGSG (SEQ ID NO: 6), SGRSSGSGSG (SEQ ID NO: 7), and/or TSGGSSGSSLVQHPLF (SEQ ID NO: 8), SGSSSGSGFR (SEQ ID NO: 9), SDSSSGSGSG (SEQ ID NO: 10), and/or fragments thereof. In some preferred embodiments one or more optional polyoxyalkelene spacer moieties are covalently bound to the peptide or mimetic thereof. The polyoxyalkelene moiety may, in some instances, be polyetheleneglycol. In further embodiments the polyoxyalkelene moiety is polyetheleneglycol, e.g., NH-PEG₂-CO—, or NH-PEG₅-CO—. One of skill in the art will appreciate that the polyoxyalkelene may be attached to a peptide of the invention at amino acid resides other than the N or C terminus and may or may not be attached to additional chemical groups. The polyoxyalkelene may be attached to one or both of the strings Z₁ and Z₂, to the peptide in the absence of Z₁ and/or Z₂, or to a functional combination thereof. The polyoxyalkelene moiety may serve as a linker group, e.g., by linking Z₁ and/or Z₂ to a peptide, or by linking Z₁ and/or Z₂ to a chemical group. The polyoxyalkelene moiety may further be attached at the end of the peptide, at the end of (or within) a chemical group, or to an amino acid side chain.

In some embodiments, chemical groups, Φ₁ and Φ₂, are attached to a peptide or mimetic thereof of the invention. Φ₁ and Φ₂, are each independently absent or are independently chosen chemical groups covalently bound to the peptide or mimetic thereof, to one or both of the strings Z₁ and Z₂, to the one or more polyoxyalkelene moieties, or to a functional combination thereof. In preferred embodiments the chemical groups may be chosen from the group comprising or consisting of —NH2, —N-biotinyl-K—CO—NH₂, wherein K is the D or L type optical isomer of Lysine, and —NH-(PEG)-COOH, wherein n is any integer from 1 to 10,000, and a detectable label.

In some embodiments the peptides of the invention may include conservative amino acid modifications or conservative amino acid substitutions. In further embodiments, peptides of the invention are those which retain at least 50% homology or sequence identity to the specific peptides disclosed herein (e.g., sequences 07-007(GNALHWVCASNICWRSPWAGRLWGLVRLT (SEQ ID NO: 11)), 07-071(SGSSSGSGSGNTLHWVCASDICWRTPWAGQLWGLVRLT (SEQ ID NO: 12)) or fragments of 07-071 (e.g., NTLHWVCASDICWRTPWAGQLWGLVRLT (SEQ ID NO: 13); SGSSSGSGSGNTLHWVCASDICWRTPWAGQLWGLVRL (SEQ ID NO: 14); NTLHWVCASDICWRTPWAGQLWGLVRL (SEQ ID NO: 15), etc.), 07-072(SGSSSGSGSGNALHWVCASNICWRTPWAGQLWRLVRLT (SEQ ID NO: 16)) or fragments of 07-072 (e.g. NALHWVCASNICWRTPWAGQLWRLVRL (SEQ ID NO: 17); NALHWVCASNICWRTPWAGQLWRLVRLT (SEQ ID NO: 18); SGSSSGSGSGNALHWVCASNICWRTPWAGQLWRLVRL (SEQ ID NO: 19), etc.) G2306 (NALHWVCASNICWRSPWAGRLWGLVRL (SEQ ID NO: 20)), or G2257(NALHWVCASNICWRSPWAGRLWGLVRL (SEQ ID NO: 20))).

In specific embodiments the isolated peptide or mimetic thereof is 07-071 or fragments or conservative amino acid modifications thereof. In a similar embodiments the isolated peptide or mimetic thereof is 07-072 or fragments or conservative amino acid modifications thereof. In other embodiments the isolated peptide or mimetic thereof is G2306 or fragments or conservative amino acid modifications thereof. In additional embodiments the isolated peptide or mimetic thereof is 37X (NALHWVCASNICWRTPWAGRLWGLVRL (SEQ ID NO: 21)), 29X(NALHWVCASNICWRTPWAGQLWGLVRL (SEQ ID NO: 22)), 14X(NALHWVCASNICWRTPWAGRLWRLVRL (SEQ ID NO: 23)), 8X(NALHWVCASNICWRTPWAGRLWELVRL (SEQ ID NO: 24)), (VQEDVSSTLGSWVLLPFHRGTRLSVWVT (SEQ ID NO: 25)), (GGFEGLSQARKDQLWLFLMQHIRSYRTIT (SEQ ID NO: 26)), or fragments and/or modifications thereof.

In a similar embodiment an isolated peptide or mimetic thereof is provided, wherein the peptide comprises the formula S-X₁-T-L-X₂-S-X₃-V-X₅ (SEQ ID NO: 27) wherein X₁ is any amino acid, X₂ is any amino acid, X₃ is chosen from the group consisting of the amino acids W and F, and X₅ is chosen from the group consisting of the amino acids L and I. In related embodiments the isolated peptide or mimetic thereof is

| | |
|---|---|
| G2211/2226 | |
| (GVQEDVSSTLGSWVLLPFHRGTRLSVWVT, | (SEQ ID NO: 28)) |
| G2212/2227 | |
| (GAGLWWGFCTDQHCIFKSPTLSSFVIVDT, | (SEQ ID NO: 29)) |
| G2255/2256 | |
| (GGFEGLSQARKDQLWLFLMQHIRSYRTIT, | (SEQ ID NO: 26)) |
| G2257/2258 | |
| (07-D60 | (SEQ ID NO: 30)) |
| GNALHWVCASNICWRPPWAGRLWGLVRLT | |

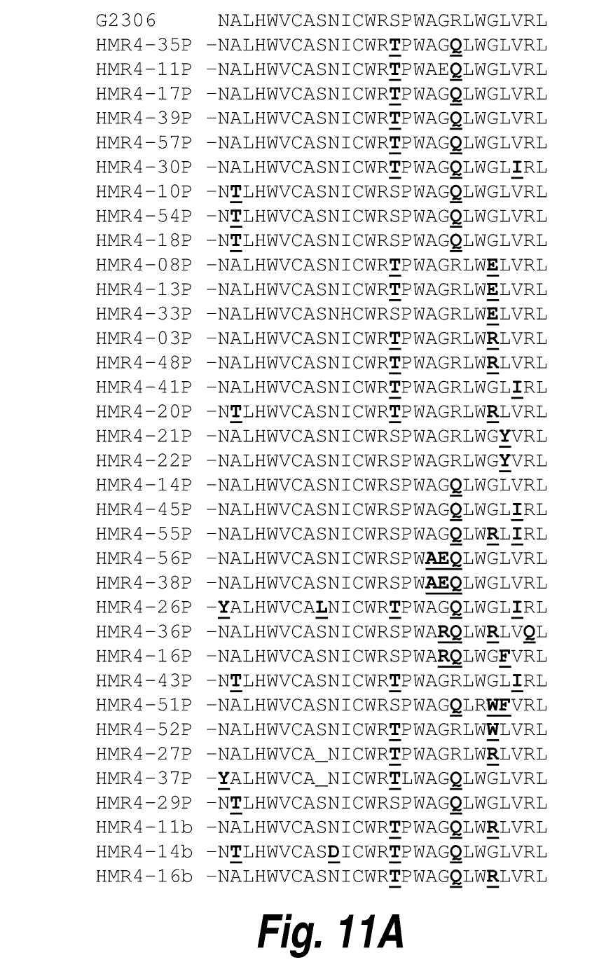

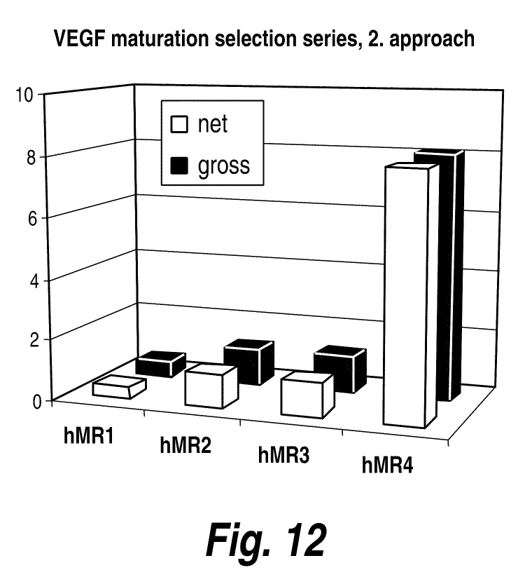

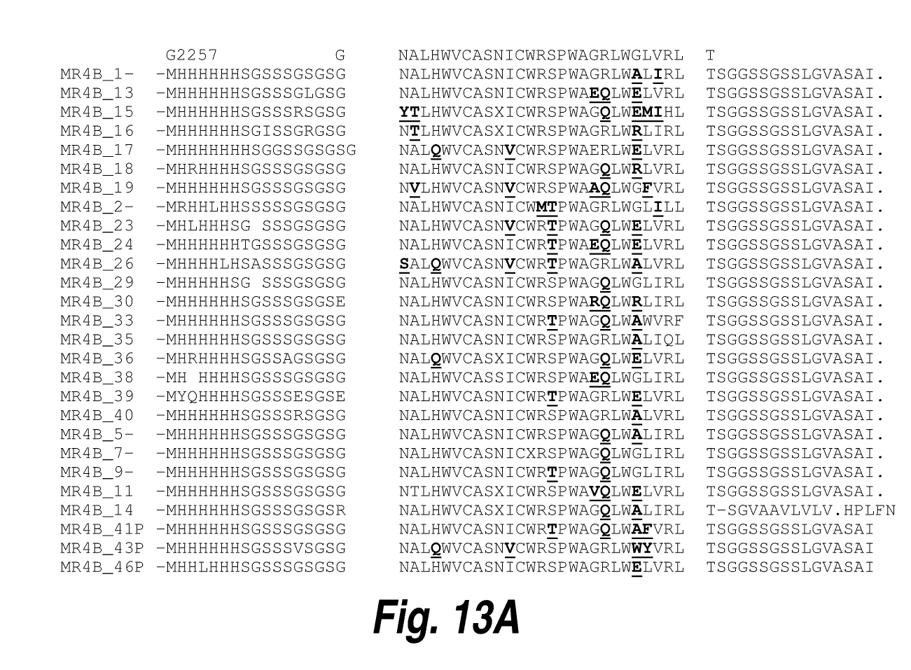

or fragments/modifications thereof. In some embodiments the isolated peptide or mimetic thereof is any of the peptides listed in Table 2, Table 3, FIG. 11, FIG. 13, FIG. 14 or fragments and/or modifications thereof.

In a related aspect the isolated peptide or mimetic thereof of the invention specifically binds to VEGF with a KD selected from the group consisting of 1×10⁻⁶ M or less.

A further aspect of the invention provides a method of treating a VEGF modulated disease in a subject, comprising administering to the subject an effective amount of an isolated peptide or mimetic thereof of the invention, thereby treating the VEGF modulated disease. In specific embodiments the VEGF modulated disease is at least one of cancer, macular degeneration, diabetic retinopathy, psoriasis, diabetes, cardiovascular ischemia, rheumatoid arthritis, osteoarthritis, or any of the diseases described herein.

In a further aspect of the invention, methods are provided to detect VEGF in a biological sample. In one embodiment the method for detecting VEGF in a biological sample, comprises (a) incubating a biological sample with a peptide or mimetic thereof which specifically binds to VEGF wherein the amino acids in said peptide or mimetic thereof are D type optical isomers and wherein said incubation allows the formation of a complex between VEGF and said peptide or mimetic thereof; and (b) detecting VEGF bound to the immobilized capture reagent. Further embodiments include measuring an amount of VEGF detected in the sample, wherein the amount is quantitated using a standard curve. Methods for detecting VEGF may include, but are not limited to ELISA assays, BiaCORE assays, immunological assays, fluorescence assays, FRET and BRET assays. In preferred embodiments, a peptide or mimetic thereof of the invention contains a fluorescent label such that it may be detected upon binding to VEGF. As such, VEGF may be detected or quantitated using the peptides or mimetics thereof of the invention. In specific embodiments the biological sample is isolated from a human. In further embodiments the human has vascular disease, diabetes, cancer, or macular degeneration. In some embodiments the biological sample is blood, plasma, serum, urine, a tissue biopsy, a tumor sample, or any specimen which may contain VEGF or exhibit angiogenesis. In some embodiments, the detection of VEGF in a biological sample may be indicative of a VEGF modulated disease in a subject and thus may be used for diagnostic purposes, e.g., where in increased or decreased amount of VEGF is diagnostic for a VEGF modulated disease.

### DESCRIPTION

This specification describes, inter alia, the identification and production of novel, polypeptides that bind to VEGF-1. VEGF-1 is a potent pro-angiogenic cytokine which is involved in several normal and disease conditions. Binding of VEGF-1 to its cellular receptor initiates a signal transduction cascade that is critical to diverse processes such as wound healing, ovulation, tumor growth, and rheumatoid arthritis. VEGF-1 exists in at least four isoforms generated by splicing at the nucleic acid level with 121, 145, 165, 189 and 206 amino acids. All of the isoforms are capable of binding to and activating VEGFR-1 and VEGFR-2, but differ in their binding to cell-surface heparin sulfates and the extracellular matrix (ECM). VEGF121 is a freely diffusible protein, while the larger isoforms appear to become immobilized by heparin and ECM binding in vivo. All VEGF-1 isoforms are homodimers covalently joined by intermolecular disulfide bonds. Furthermore, all VEGF-1 isoforms appear to share a common receptor binding cysteine-knot domain which is contained within residues 8-109. This domain has been structurally characterized by both NMR and X-ray crystallographic methods. A detailed review of VEGF, its role in normal tissue, and its contribution to disease is available in Hoeben et al. (2004) Pharmacological Reviews. 56(4):549-580. A number of avenues to inhibition of VEGF signaling are being pursued, including decoy receptors (VEGF-Trap, Regeneron), modified nucleic acid aptamers (Macugen, Eyetech/Pfizer) antibodies directed against VEGF receptors, antisense induced downregulation of VEGF and its receptors and antiangiogenic ribozymes (Angiozyme, Ribozyme Pharmaceuticals) but the best characterized system remains direct blockade of VEGF signaling by monoclonal antibody (Avastin, Genentech).

The present specification demonstrates, in part, library screening to identify D-peptides that bind to the pro-angiogenic cytokine VEGF, and to select D-peptides that can block cytokine-mediated signaling for development as therapeutics for AMD, oncological indications, and other disease conditions.

The present disclosure provides polypeptides that bind to VEGF, many of which exhibit in vitro and/or in vivo VEGF antagonist activity. Polypeptides having VEGF antagonist activity will be useful in numerous therapeutic applications. Anti-VEGF therapies have been established as having in vivo utility against diseases and conditions ranging from cancers and complications resulting from cancers to proliferative retinopathies, inflammatory disorders and fibrosis. Based on the in vivo and in vitro data presented here, it is expected that the peptides or mimetics thereof of the invention will be useful in treating the same spectrum of disorders.

In addition to therapeutic applications, VEGF-binding peptides and mimetics thereof may be used in circumstance where it is desirable to detect VEGF. Such diagnostic applications are described below in Section VI.

The inventors isolated from a library, peptides that bind to VEGF, wherein, in some instances, the D type optical isomers of said peptides inhibit VEGF biological activities. As discussed above, it is expected that peptides having certain desirable properties, such as high affinity binding to VEGF, can be used as effective therapeutic agents, e.g., anti-cancer agents. While it is expected that the effectiveness of such polypeptides as therapeutic agents is related to their effect on angiogenesis, we do not wish to be bound to any particular mechanism. It is formally possible that the present peptides or mimetics thereof act as effective therapeutics for reasons unrelated to angiogenic processes. In the alternative, the VEGF binding peptides or mimetics thereof of the present invention may, in some embodiments, bind VEGF without inhibiting VEGF biological activity. Peptides which do not inhibit VEGF biological activity may bind VEGF and still allow binding of VEGF to the VEGF receptor. Such binding may be useful for diagnostic purposes, e.g., by attaching a label to a peptide of the invention and imaging cells which express VEGF-receptor (or to deliver secondary therapeutics as described herein).

The term “inhibit” is meant to convey a measurable reduction in a phenomenon, often used herein in reference to any of the following: the interaction of VEGF with a VEGF receptor, VEGF- or VEGFR-mediated angiogenesis, angiogenesis, symptoms of angiogenesis, the viability of VEGFR-containing cells, the viability of VEGF-dependent Ba/F3 cells, or VEGF- or VEGFR-mediated cellular proliferation as compared to a control sample not treated with the peptide or mimetic thereof of the invention. A peptide or mimetic thereof of the invention will inhibit a VEGF- or VEGFR mediated activity if the reduction in activity or interaction is at least 5%, preferably 10%, 20%, 30%, 40%, or 50%, and more preferably 60%, 70%, 80%, 90% or more.

By “VEGF biological activity” is meant any function of any VEGF family member acting through any VEGF receptor, but particularly signaling through a VEGFR-2 receptor. The VEGF family includes VEGF-A, VEGF-B, VEGF-C, VEGF-D, VEGF-E, VEGF-F, and placental growth factor (PIGF), as well as various alternatively spliced forms of VEGF including VEGF121, VEGF145, VEGF165, VEGF189, and VEGF206 (Tischer et al. 1991. J. Biol. Chem., 266: 11947-11954; Hoeben et al. 2004. Pharmacological Reviews, 56:549-580, 2004). The VEGFR family of tyrosine kinase receptors includes VEGFR-1 (also known as Flt-1), VEGFR-2 (also known as KDR (human form) or Flk-1 (mouse form)), and VEGFR-3 (also known as Flt-4). VEGF ligands bind to the VEGF receptors to induce, for example, angiogenesis, vasculogenesis, endothelial cell proliferation, vasodilation, and cell migration. VEGF ligands can also inhibit apoptosis through binding to their cognate receptors. VEGFR-2 is believed to be the VEGFR most involved in angiogenesis. A VEGFR-2 or KDR-mediated biological activity is any biological function in which VEGFR-2 or KDR participates in significantly, such that antagonism of VEGFR-2 or KDR causes a measurable decrease in the biological activity. The biological activity of VEGF and VEGFR can be measured by standard assays known in the art. Examples include ligand binding assays and Scatchard plot analysis; receptor dimerization assays; cellular phosphorylation assays; tyrosine kinase phosphorylation assays (see for example Meyer et al., Ann N.Y. Acad. Sci. 995: 200-207, 2003); endothelial cell proliferation assays such as BrdU labeling and cell counting experiments; VEGF-dependent cell proliferation assays; and angiogenesis assays. Methods for measuring angiogenesis are standard, and are described, for example, in Jain et al. (Nat. Rev. Cancer 2: 266-276, 2002). Angiogenesis can be assayed by measuring the number of non-branching blood vessel segments (number of segments per unit area), the functional vascular density (total length of perfused blood vessel per unit area), the vessel diameter, the formation of vascular channels, or the vessel volume density (total of calculated blood vessel volume based on length and diameter of each segment per unit area). Exemplary assays for VEGF-mediated proliferation and angiogenesis can be found in U.S. Pat. No. 6,559,126, Lyden et al., Nature Medicine 7: 1194 (2001), Jacob et al., Exp. Pathol. 15: 1234 (1978) and Bae et al., J. Biol. Chem. 275: 13588 (2000). These assays can be performed using either purified receptor or ligand or both, and can be performed in vitro or in vivo. These assays can also be performed in cells using a genetically introduced or the naturally-occurring ligand or receptor or both. A peptide or mimetic thereof of the invention that inhibits the biological activity of VEGF will cause a decrease of at least 5%, preferably 10%, 20%, 30%, 40%, or 50%, and more preferably 60%, 70%, 80%, 90% or greater decrease in the biological activity of VEGF. The inhibition of biological activity can also be measured by the IC50. Preferably, in some embodiments, a peptide or mimetic thereof of the invention that inhibits the biological activity of VEGF or VEGFR-2 will have an IC50 of less than 100 nM, more preferably less than 10 nM and most preferably less than 1 nM.

### I. Peptides

The methodology described herein has been used to develop VEGF binding peptides or mimetics thereof. By a “peptide” is meant any sequence of two or more amino acids, regardless of length, post-translation modification, or function. “Polypeptide,” “peptide,” and, “protein” are used interchangeably herein. The peptides of the invention are of limited size, and do not generally rely on three-dimensional folding into domains of large amino acid sequences for their activity. The peptides or mimetics thereof of the invention are typically, though not universally, between 4 and 90 amino acids in length. In various embodiments a peptide of the invention may be less than 200 amino acids in length, less than 180 amino acids in length, less than 160 amino acids in length, less than 140 amino acids in length, less than 120 amino acids in length, less than 100 amino acids in length, less than 90 amino acids in length, less than 80 amino acids in length, less than 70 amino acids in length, less than 60 amino acids in length, less than 50 amino acids in length, less than 40 amino acids in length, less than 35 amino acids in length, less than 30 amino acids in length, less than 28 amino acids in length, less than 27 amino acids in length, 25 amino acids in length, less than 20 amino acids in length, less than 18 amino acids in length, less than 15 amino acids in length, less than 10 amino acids in length, or about 4 or 5 amino acids in length.

The peptides of the invention can include natural amino acids and non-natural amino acids such as those described in U.S. Pat. No. 6,559,126, incorporated herein by reference. The peptides of the invention may be composed of one or more, or most preferably all, amino acids which are D-type optical isomers. Nineteen of the essential twenty amino acids have the property of “chirality” or handedness. The only achiral essential amino acid is glycine. To describe a chiral compound, the prefixes D and L are used to refer to the configuration of the molecule around its chiral center. The chiral center of an amino acid is the alpha carbon, and whether an amino acid is of the D configuration or the L configuration depends upon the stereoisomeric conventions established by Emil Fisher. The preferred D-peptide therapeutics (DRx) approach enables the selection of small D-peptides that, like antibody-based drugs, derive their therapeutic effect by selectively targeting a key element associated with a disease. However, D-peptide-based products have several advantages with respect to antibodies and other protein therapeutics. The smaller size and greater stability of the D-peptides makes them simpler to formulate for pulmonary, topical and oral delivery. D-amino acid proteins are known to be poor immunogens, and the D-peptide compounds of the invention behave accordingly (Dintzis et al. (1993) PROTEINS: Structure, Function, and Genetics 16, 306-308). Their resistance to enzymatic degradation, and their ability to be combined with polymers, results in enhanced pharmacokinetics compared to other peptide drugs. Also, the final D-peptides have reduced manufacturing costs that could be passed on to the consumer.

D-peptide-based therapeutics have significant formulation advantages over their antibody counterparts. Small peptides, unlike other types of protein therapeutics that rely on a complex folded structure for activity, are not susceptible to thermal denaturation. Thus, shelf-stable forms of D-peptides are readily envisioned. The increased in vivo stability of the D-peptides of the invention also allows the design of inhalable, topical, and orally available formulations not possible for antibodies. Evidence for the advantages of D-peptide therapeutics is available in the art. For example, RDP58, a peptide composed of non-natural and D-amino acids, is in Phase II clinical development for IBD as an orally administered agent acting topically on the intestinal lumen. This product is an inhibitor of several cytokines including TNF, but its lack of specificity may make it less attractive than a more potent D-peptide alternative (Grassy et al. (1998), Nat Biotechnology 16, p. 748-752).

The ability to engineer the structure of D-peptides, as described herein, provides advantages over antibody-based drugs. For example, the methods of the present invention allow for the synthesis of multivalent D-peptide constructs by using well-defined linker chemistries to tune the affinities of the D-peptides, and design D-peptide based receptor agonists or antagonists. Multivalent constructs of L-peptides derived from phage display screening against cytokine receptors can exhibit potent agonist activity (Cwirla et al. (1997) Science 276, 1696-1699; Wrighton et al. (1996) Science 273, 458-464). However, the drawbacks of L-peptides have hindered the development of these tantalizing leads into successful drugs. Unlike antibody-based molecules, it is expected that next-generation multivalent D-peptide constructs can activate or inhibit therapeutically important receptors without the drawbacks of L-peptide counterparts.

Market availability of the D-peptides of the invention will prove to be a key therapeutic benefit. For example, patients have been rationed in the early years after market introduction for doses of Enbrel® due to manufacturing capacity constraints, and there is currently a bottleneck for the numerous proteins in development. Using peptide synthesis to generate D-peptides of the invention provides another manufacturing source of therapeutics to replace antibodies. Advances in supply chemistry have made thirty-six amino acid polymers available in metric tons (e.g. T-20/Fuzeon™ from Trimeris and Hoffmann-LaRoche). Furthermore, the costs of production for the D-amino acid peptide therapeutics is significantly less than those for the corresponding antibodies.

The peptides and mimetics thereof of the invention can also be modified by any variety of standard chemical methods (e.g., an amino acid can be modified with a protecting group; the carboxy-terminal amino acid can be made into a terminal amide group; the amino-terminal residue can be modified with groups to, e.g., enhance lipophilicity; or the polypeptide can be chemically glycosylated or otherwise modified to increase stability or in vivo half-life). Modifications and derivatives of the peptides of the invention are discussed in further detail in Section IV below.

The peptides or mimetics thereof of the invention include single amino acid chains and are different from, for example, antibodies and single chain antibodies, where antigen binding activity is generally contributed by two peptide chains (or a single, folded chain), a heavy chain variable domain and a light chain variable domain. It is contemplated that a plurality of peptides of the sort disclosed herein could be connected to create a composite molecule with increased avidity. Likewise, a peptide may be attached (e.g., as a fusion protein) to any number of other polypeptides, such as fluorescent polypeptides, targeting polypeptides and polypeptides having a distinct therapeutic effect. Peptides or mimetics thereof of the invention may be designed to include chemical modifications or particular amino acid sequences which promote solubility. For example, in some embodiments peptides were synthesized to include the amino acids DDD or KKK in the N-terminal or C-terminal regions. Although in the most preferred embodiments, the peptides of the invention are D-type amino acids, any additional amino acids attached to the functional portion (i.e., the portion responsible for binding to VEGF) of a peptide of the invention may be either L or D-type optical isomers (e.g., the DDD or KKK peptides described above may be L-type optical isomers while the rest of the peptide contains D-type optical isomers).

By “specifically binds” is meant that a peptide recognizes and interacts with a target protein (e.g., VEGF) but that does not substantially recognize and interact with other molecules in a sample, e.g., a biological sample. In preferred embodiments a peptide of the invention will specifically bind a VEGF with a KD at least as tight as 1×10⁻³ M. Preferably, the polypeptide will specifically bind a VEGF with a KD of 1×10⁻³ M to 1×10⁻⁹ M, more preferably 1×10⁻⁶ M, 1×10⁻⁷ M, or lower.

In some embodiments, preferred peptides or mimetics thereof of the invention which bind VEGF have a dissociation constant to VEGF165 of 30 nM or less, more preferably 25 nM or less, 20 nM or less, 15 nM or less, 12 nM or less, 10 nM or less, 8 nM or less, 6 nM or less, or 4 nM or less.

In some embodiments, preferred peptides or mimetics thereof of the invention inhibit the growth of HUVEC cells with an inhibition constant of 700 nM or less, more preferably 500 nM or less, 400 nM or less, 300 nM or less, 200 nM or less, 150 nM or less, or even more preferably 100 nM or less, 90 nM or less, 80 nM or less, 70 nM or less, 60 nM or less, 50 nM or less, 40 nM or less, 30 nM or less, 20 nM or less, or 10 nM or less.

In some embodiments, preferred peptides or mimetics thereof of the invention block blood vessel growth in the corneal micropocket model, within 300 fold of the effect observed when Avastin is used, or within 200-, 100-, or 50-fold of the effect observed when Avastin is used. In some embodiments, preferred peptides or mimetics thereof of the invention block blood vessel growth in the ocular implant model within 200-fold of Macugen's effect, or in some embodiments within 100-, 75-, 50-, 20-, or 10-fold of Macugen's effect.

The present invention also encompasses “conservative sequence modifications” or “conservative amino acid modifications” of the sequences described herein, i.e., amino acid sequence modifications which do not significantly affect or alter the binding characteristics of the peptide encoded by the nucleotide sequence or containing the amino acid sequence. Such conservative sequence modifications include nucleotide and amino acid substitutions, additions and deletions. Modifications can be introduced into sequences by standard techniques known in the art, such as site-directed mutagenesis and PCR-mediated mutagenesis. In some embodiments, the modifications are chosen by rational design, and the designed peptides are generated by chemical synthesis as described herein. “Conservative amino acid modifications” includes conservative amino acid substitutions which are substitutions in which the amino acid residue is replaced with an amino acid residue having a similar side chain (e.g., similar size, shape, electric charge, chemical properties including the ability to form covalent or hydrogen bonds, or the like). Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). A peptide or mimetic thereof of the invention may be modified by one or more substitutions, particularly in portions of the protein that are not expected to interact with a target protein. It is expected that as many as 5%, 10%, 20%, 30%, 40%, 50%, or even 50% or more of the amino acids in peptide may be altered by a conservative substitution without substantially altering the affinity of the protein for target. It may be that such changes will alter the immunogenicity of the polypeptide in vivo, and where the immunogenicity is decreased, such changes will be desirable. Further non-limiting examples of homologous substitutions that can be made in the structures of the peptidic molecules of the invention include substitution of D-phenylalanine with D-tyrosine, D-pyridylalanine or D-homophenylalanine, substitution of D-leucine with D-valine or other natural or non-natural amino acid having an aliphatic side chain and/or substitution of D-valine with D-leucine or other natural or non-natural amino acid having an aliphatic side chain. In some embodiments, conservative amino acid substitutions alone, i.e., without amino acid deletions or additions are the preferred type of amino acid modification. One of skill in the art will appreciate that such modifications or substitutions may be made at the DNA level, thus encoding the altered or substituted peptide, or they may be made at the protein level, e.g., by direct chemical synthesis.

In some embodiments the peptides of the invention may be cyclized. Such “cyclic peptides” have intramolecular links which connect two amino acids. Cyclic peptides are often resistant to proteolytic degradation and are thus good candidates for oral administration. The intramolecular linkage may encompass intermediate linkage groups or may involve direct covalent bonding between amino acid residues. In some embodiments, the N-terminal and C-terminal amino acids are linked. In other embodiments, one or more internal amino acids participates in the cyclization. Other methods known in the art may be employed to cyclize peptides of the invention. For example, cyclic peptides may be formed via side-chain Azide-Alkyne 1,3-dipolar cycloaddition (Cantel et al. J. Org. Chem., 73 (15), 5663-5674, 2008, incorporated herein by reference). Cyclization of peptides may also be achieved, e.g., by the methods disclosed in U.S. Pat. Nos. 5,596,078; 4,033,940; 4,216,141; 4,271,068; 5,726,287; 5,922,680; 5,990,273; 6,242,565; and Scott et al. PNAS. 1999. vol. 96 no. 24 P. 13638-13643, which are all incorporated herein by reference. In some embodiments the intramolecular link is a disulfide bond mimic or disulfide bond mimetic which preserves the structure that would be otherwise be created by a disulfide bond.

In some particularly preferred embodiments, the cyclization of the peptides occurs via intramolecular disulfide bonds. In some preferred embodiments, the formation of an intramolecular disulfide bond increases the affinity of the peptide for VEGF. Accordingly, the methodology used to select and/or affinity mature the peptides or mimetics thereof of the invention may be performed under conditions which allow disulfide bond formation prior to and during selection (e.g., oxidizing conditions). In some particularly preferred embodiments the disulfide bonds may form between cysteine residues which naturally exist in the library or peptide, or which are introduced by the mutation process during one or more rounds of selection. In other embodiments the peptides may be designed to contain cysteine residues at particular positions such that it is known which residues participate in the disulfide bond. Intramolecular disulfide bonding between cysteine residues may be induced by methods known in the art (e.g., U.S. Pat. Nos. 4,572,798; 6,083,715; 6,027,888, and WIPO Publication WO/2002/103024 which are incorporated herein by reference).

In some embodiments, the formation of a disulfide bond (or the formation of a cyclized or intramolecularly linked structure in general) imparts a particular structure onto the peptide which is important for target binding. Accordingly, the disulfide bonds and/or cyclization preferably form prior to peptide selection such that the potentially favorable structure created by bond formation may be selected for. In some embodiments the peptides or mimetics thereof of the invention may have more than one, two, three, or more disulfide bonds. Further methods known in the art to generate, and select peptides with intramolecular di-sulfide bonds, intramolecular di-sulfide bond substitutes, and other intramolecular links may be employed. For example, the methods described in WO03040168, incorporated herein by reference, describe methods to generate and select peptide apatamers, conotides, and other cyclic peptides which, in some embodiments, may be employed with the methods of the present invention.

In related embodiments a peptide conformation or structure which is beneficial to binding (e.g., it increases binding affinity to VEGF) may be preserved or mimicked by chemical crosslinking or other methods of peptide stabilization. For example, a beneficial peptide conformation or structure which is formed by disulfide bonds may be stabilized by chemical treatment or reaction, thus allowing the preservation of the structure without a disulfide bond. Indeed, peptide stabilization techniques may be employed to stabilize peptides of the invention whether or not a disulfide bond was originally present. For example, the techniques described in Jackson, et al. J. Am. Chem. Soc. 1991, 113, 9391-9392; Phelan, et al. J. Am. Chem. Soc. 1997, 119, 455-460; Bracken, et al. J. Am. Chem. Soc. 1994, 116, 6431-6432, which are incorporated herein by reference, may be used to stabilize peptides of the invention.

Other methods to stabilize peptides and peptide structures may be used, e.g., olefinic cross-linking of helices through O-allyl serine residues (Blackwell, H. E.; Grubbs, R. H. Angew. Chem., Int. Ed. 1998, 37, 3281-3284, incorporated herein by reference), all-hydrocarbon cross-linking (Schafmeister and Verdine J. Am. Chem. Soc. 2000, 122 (24), 5891-5892, incorporated herein by reference) and the methods disclosed in U.S. Pat. No. 7,183,059 (incorporated herein by reference). The methods disclosed in Blackwell et al. and Schafmeister et al. may be described as producing “stapled” peptides, i.e., peptides which are covalently locked into a particular conformational state or secondary structure, or peptides which have a particular intramolecular covalent linkage which predisposes them to form a particular conformation or structure. If a peptide thus treated is predisposed to, e.g., form an alpha-helix which is important for target binding, then the energetic threshold for binding will be lowered. Such “stapled” peptides have been shown to be resistant to proteases and may also be designed to cross the cellular membrane more effectively (also see Walensky et al. Science 2004:Vol. 305. no. 5689, pp. 1466-1470; Bernal et al. J Am Chem Soc. 2007, 129(9):2456-7 which are incorporated herein by reference). Accordingly, peptides of the invention may be thus stapled or otherwise modified to lock them into a specific conformational shape or they may be modified to be predisposed to particular conformation or secondary structure which is beneficial for binding. It is contemplated that such peptide modifications may occur prior to peptide selection such that the benefit of any conformational constraints may also be selected for. Alternatively, in some embodiments, the modifications may be made after selection to preserve a conformation known to be beneficial to binding or to further enhance a peptide candidate.

Also included in the present invention are nucleic acid sequences encoding any of the peptides described herein. As appreciated by those skilled in the art, because of third base degeneracy, almost every amino acid can be represented by more than one triplet codon in a coding nucleotide sequence. In addition, minor base pair changes may result in a conservative substitution in the amino acid sequence encoded but are not expected to substantially alter the biological activity of the gene product. Therefore, a nucleic acid sequence encoding a polypeptide described herein may be modified slightly in sequence and yet still encode its respective gene product.

In addition, the peptides of the present invention can be used as lead peptides that can be further mutated, altered, and screened for peptides that bind VEGF with an even greater affinity. In one example, a peptide described herein is used as a lead polypeptide which is further altered to produce peptides with amino acid changes distinct from the lead polypeptide. The further altered peptides can then be used to screen for those that bind and/or inhibit VEGF biological activity. Finally, the peptides or mimetics thereof of the invention may be modified as described below in section 4 (Modification of Peptides).

The present disclosure describes many peptides which have been selected to bind VEGF. For example, Table 2, Table 3, FIG. 11, FIG. 13, and FIG. 14 disclose particular peptides of the invention. One of skill in the art will appreciate that motifs discovered in the selected peptides may serve as the basis for further peptides that bind VEGF. For example, sequences selected from libraries derived from G2211/G2226 were found to include the motif S-X₁-T-L-X₂-S-X₃-V-X₅ (SEQ ID NO: 27), wherein X₁ is any amino acid, X₂ is any amino acid, X₃ is W or F, and X₅ is L or I. Additionally, observation of the variability of peptides selected in Examples 11 and 12 (shown in FIG. 11 and FIG. 13) shows that peptides of the invention may encompass a wide variety of sequences and may be composed of, in some preferred embodiments, peptides which are described by the following formula:

G-X₁-X₂-L-X₃-X₄-V-C-X₅-X₆-X₇-X₈-C-W-X₉-X₁₉-X₁₁-W-A-X₁₂-X₁₃-X₁₄-X₁₅-X₁₆-X₁₇-X₁₈-X₁₉-L (SEQ ID NO: 31) wherein X₁ is one of the amino acids N, Y, F, D, I, or H, X₂ is one of the amino acids A, T, or V, X₃ is one of the amino acids H, Q, or R, X₄ is one of the amino acids W or R, X₅ is one of the amino acids A or V, X₆ is one of the amino acids S or L, X₇ is one of the amino acids N, S, or D, X₈ is one of the amino acids I, V, or H, X₉ is one of the amino acids R or M, X₁₀ is one of the amino acids S, T, P, or F, X₁₁ is one of the amino acids P or L, X₁₂ is one of the amino acids G, E, R, A, or V, X₁₃ is one of the amino acids R, or Q, X₁₄ is one of the amino acids L or W, X₁₅ is one of the amino acids W or R, X₁₆ is one of the amino acids G, R, E, A, V, or W, X₁₇ is one of the amino acids L, F, M, W, or Y, X₁₈ is one of the amino acids V or I, and X₁₉ is one of the amino acids R, L, Q, or H.

In still another preferred embodiment, a peptide or mimetic thereof of the invention comprises a peptide sequence having at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more identity to any of the peptide sequences shown in FIG. 11, FIG. 13, FIG. 14, Table 2, Table 3 or to G2211/2226 or G2212/2227, or a portion thereof. In a particularly preferred embodiment the invention comprises a peptide sequence having at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more identity to the peptide 07-072 or 07-071.

Ranges and identity values intermediate to the above-recited ranges, (e.g., 70-90% identical or 80-95% identical) are also intended to be encompassed by the present invention. For example, ranges of identity values using a combination of any of the above values recited as upper and/or lower limits are intended to be included. Also encompassed by the invention are nucleic acid sequences (and complements thereof) which code for the peptides disclosed herein, inclusive of peptides described above by sequence identity.

The comparison of sequences and determination of percent identity (or homology) between two sequences are art-known techniques, and can be accomplished using a mathematical algorithm, such as the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST, XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences similar to nucleic acid molecules of the invention. BLAST protein searches can be performed with the XBLAST program, score=50, wordlength=3 to obtain amino acid sequences homologous to peptides of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, one of ordinary skill in the art will know how to optimize the parameters of the program (e.g., XBLAST and NBLAST) for the specific sequence being analyzed. Moreover, the BLASTP program, which is designed on principles similar to NBLAST and XBLAST, may be easily used to determine identity between peptide sequences.

Another example of a mathematical algorithm utilized for the comparison of sequences is the algorithm of Meyers and Miller ((1988) Comput. Appl. Biosci. 4: 11-17). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art, and include ADVANCE and ADAM described in Torelli and Robotti (1994) Comput. Appl. Biosci. 10:3-5; and FASTA, described in Pearson and Lipman (1988) P.N.A.S. 85:2444-8.

The percent identity (or homology) between two amino acid sequences can also be accomplished using the GAP program in the GCG software package (available at the Accelrys™ website), using either a Blosum 62 matrix or a PAM250 matrix, and a gap weight of 12, 10, 8, 6, or 4 and a length weight of 2, 3, or 4. The percent identity between two nucleic acid sequences can be accomplished using the GAP program in the GCG software package, using standard parameters, such as a gap weight of 50 and a length weight of 3.

Alternatively, the SIM alignment tool may be used to align protein sequences (expasy.org/tools/sim-prot.html) using either a Blosum matrix (e.g., Blosum30, Blosum62, Blosum100) or a PAM matrix (PAM40, PAM120, PAM200, PAM250, or PAM400), and a gap weight of 12, 10, 8, 6, or 4 and a length weight of 2, 3, or 4.

### II. Nucleic Acids and Production of Polypeptides of the Present Invention can be Produced Using any Standard Methods Known in the Art.

Peptides of the invention which are L-type optical isomers may be produced by the standard methods known in the art. For example, peptides may be produced by recombinant DNA methods, inserting a nucleic acid sequence (e.g., a cDNA) encoding the polypeptide into a recombinant expression vector and expressing the DNA sequence under conditions promoting expression. General techniques for nucleic acid manipulation are described for example in Sambrook et al., Molecular Cloning: A Laboratory Manual, Vols. 1-3, Cold Spring Harbor Laboratory Press, 2 ed., 1989, or F. Ausubel et al., Current Protocols in Molecular Biology (Green Publishing and Wiley-Interscience: New York, 1987) and periodic updates, herein incorporated by reference. Appropriate cloning and expression vectors for use with bacterial, fungal, yeast, and mammalian cellular hosts can be found in Cloning Vectors: A Laboratory Manual, (Elsevier, N.Y., 1985), the relevant disclosure of which is hereby incorporated by reference. Other recombinant DNA methods are described in U.S. Pat. Nos. 4,356,270 4,399,216, 4,506,013, 4,503,142, 4,952,682, 5,618,676, 5,854,018, 5,856,123, 5,919,651, and 6,455,275, which are all incorporated herein by reference.

Peptides consisting partly or completely of D amino acids do not generally occur naturally. Accordingly, the preferred peptides, which are D-type optical isomers, are generally made by chemical synthesis, using techniques that are well-known in the art. For example, D-peptides can be synthesized using stepwise addition of D-amino acids in a solid-phase synthesis method involving the use of appropriate protective groups. Solid phase peptide synthesis techniques commonly used for L-peptides are described by Meinhofer, Hormonal Proteins and Peptides, vol. 2, (New York 1983); Kent, et al., Ann. Rev. Biochem., 57:957 (1988); Bodanszky et al., Peptide Synthesis, (2d ed. 1976); Atherton et al. (1989) Oxford, England: IRL Press. ISBN 0199630674; Stewart et al. (1984). 2nd edition, Rockford: Pierce Chemical Company, 91. ISBN 0935940030; and Merrifield (1963) J. Am. Chem. Soc. 85: 2149-2154 all of these references are incorporated herein by reference. D-amino acids for use in the solid-phase synthesis of D-peptides can be obtained from a number of commercial sources. D-peptides and peptides that contain mixed L- and D-amino acids are known in the art. Also, peptides containing exclusively D-amino acids (D-peptides) have been synthesized. See Zawadzke et al., J. Am. Chem. Soc., 114:4002-4003 (1992); Milton et al., Science 256: 1445-1448 (1992). Additional methods to make D-peptides have been described in the art and can be found at least in WIPO Publication No. WO/1997/013522, and U.S. Application No. 60/005,508, which are both incorporated herein by reference.

The peptide of the present invention can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, the peptides may be exchanged into different buffers and/or concentrated by any of a variety of methods known to the art, including, but not limited to, filtration and dialysis. The purified polypeptide is preferably at least 85% or 90% pure, more preferably at least 93% or 95% pure, and most preferably at least 97%, 98%, or 99% pure. Regardless of the exact numerical value of the purity, the peptide is sufficiently pure for use as a pharmaceutical product.

### III. Modification of Peptides

Approaches to designing peptide analogs, derivatives and mimetics are known in the art. For example, see Farmer, P. S. in Drug Design (E. J. Ariens, ed.) Academic Press, New York, 1980, vol. 10, pp. 119-143; Ball. J. B. and Alewood, P. F. (1990) J. Mol. Recognition. 3:55; Morgan, B. A. and Gainor, J. A. (1989) Ann. Rep. Med. Chem. 24:243; and Freidinger, R. M. (1989) Trends Pharmacol. Sci. 10:270. See also Sawyer, T. K. (1995) “Peptidomimetic Design and Chemical Approaches to Peptide Metabolism” in Taylor, M. D. and Amidon, G. L. (eds.) Peptide-Based Drug Design: Controlling Transport and Metabolism, Chapter 17; Smith, A. B. 3rd, et al. (1995) J. Am. Chem. Soc. 117:11113-11123; Smith, A. B. 3rd, et al. (1994) J. Am. Chem. Soc. 116:9947-9962; and Hirschman, R., et al. (1993) J. Am. Chem. Soc. 115:12550-12568, which are all incorporated herein by reference.

As used herein, a “derivative” of a peptidic molecule of the invention refers to a form of the peptidic molecule in which one or more reaction groups on the molecule have been derivatized with a substituent group. Examples of peptide derivatives include peptides in which an amino acid side chain, the peptide backbone, or the amino- or carboxy-terminus has been derivatized (e.g., peptidic compounds with methylated amide linkages). As used herein an “analogue” of a peptidic molecule of the invention to a peptidic molecule which retains chemical structures of the molecule necessary for functional activity of the molecule yet which also contains certain chemical structures which differ from the molecule. An example of an analogue of a naturally-occurring peptide is a peptide which includes one or more non-naturally-occurring amino acids.

As used herein, a “mimetic” of a peptidic molecule of the invention refers to a peptidic molecule in which chemical structures of the molecule necessary for functional activity of the molecule have been replaced with other chemical structures which mimic the conformation of the molecule. Examples of peptidomimetics include peptidic compounds in which the peptide backbone is substituted with one or more benzodiazepine molecules (see e.g., James, G. L. et al. (1993) Science 260:1937-1942). The term mimetic, and in particular, peptidomimetic, is intended to include isosteres. The term “isostere” as used herein is intended to include a chemical structure that can be substituted for a second chemical structure because the steric conformation of the first structure fits a binding site specific for the second structure. The term specifically includes peptide back-bone modifications (i.e., amide bond mimetics) well known to those skilled in the art. Such modifications include modifications of the amide nitrogen, the α-carbon, amide carbonyl, complete replacement of the amide bond, extensions, deletions or backbone crosslinks. Several peptide backbone modifications are known, including ψ[CH2S], ψ[CH2NH], ψ[CSNH2], ψ[NHCO], ψ[COCH2], and ψ[(E) or (Z) CH═CH]. In the nomenclature used above, ψ indicates the absence of an amide bond. The structure that replaces the amide group is specified within the brackets. In general, the use herein of the term “peptide or mimetic thereof” encompasses the peptide molecules of the invention and any modifications, derivations, and chemical alterations made thereto (e.g., peptides, peptidomimetics, peptide isosteres, peptide derivatives, and peptides containing amino acid analogs, and, in some embodiments, peptides with attached chemical groups, and linker groups).

Other possible modifications include an N-alkyl (or aryl) substitution (ψ[CONR]), or backbone crosslinking to construct lactams and other cyclic structures. Other derivatives of the modulator compounds of the invention include C-terminal hydroxymethyl derivatives, O-modified derivatives (e.g., C-terminal hydroxymethyl benzyl ether), N-terminally modified derivatives including substituted amides such as alkylamides and hydrazides.

Peptides disclosed herein may also be modified in order to improve potency, bioavailability, chemical stability, and/or efficacy. For example, within one embodiment of the invention D-amino acid peptides may be generated in order to improve the bioactivity and chemical stability of a polypeptide structure (see, e.g., Juwadi et al., J. Am. Chem. Soc. 118: 8989-8997, 1996). Lactam constraints (see Freidinger et al., Science, 210: 656-658, 1980), and/or azabicycloalkane amino acids as dipeptide surrogates can also be utilized to improve the biological and pharmacological properties of the native peptides (see, e.g., Hanessian et al., Tetrahedron 53: 12789-12854, 1997). Amide bond surrogates, such as thioamides, secondary and tertiary amines, heterocycles among others (see review in Spatola, A. F. in “Chemistry and Biochemistry of Amino Acids, Peptides and Proteins” Wenstein, B. Ed. Marcel Dekker, New York, 1983 Vol. 7, pp 267-357) can also be utilized to prevent enzymatic degradation of the polypeptide backbone thereby resulting in improved activity.

Peptides can also be modified utilizing end group capping as esters and amides in order to slow or prevent metabolism and enhance lipophilicity. Dimers of the peptide attached by various linkers may also enhance activity and specificity (see for example: Y Shimohigashi et al., in Peptide Chemistry 1988, Proceedings of the 26th Symposium on Peptide Chemistry, Tokyo, October 24-26, pgs. 47-50, 1989). For additional examples of polypeptide modifications, such as non-natural amino acids, see U.S. Pat. No. 6,559,126, which is incorporated herein by reference.

In other embodiments the peptides or mimetics thereof of the invention may be used in a modular fashion, e.g., by fusing two or more peptides or mimetics thereof of the invention or by attaching the peptides to nucleic acids or other targeting molecules. In one embodiment fusions of peptides of the invention to nucleic acids may be used to direct DNA (or RNA) aptamers to targets. In a related embodiment, an aptamer fused to a peptide of the invention may be used to direct the peptide to its intended target, or to a particular cellular structure or location. Fusion to nucleic acids or other molecules may also function to increase sensitivity of detection of VEGF by allowing it to be measured via PCR. Additionally a peptide or mimetic thereof of the invention may be fused to a second protein or nucleic acid to immobilize it on a chip.

As discussed below the peptides may also be used for diagnostic purposes, e.g., in tumor imaging by linking a detection group to a peptide of the invention, thus transporting the detection group to the tumor. The peptides or mimetics thereof of the invention may similarly be fused to other functional proteins such as antibodies, antibody fragments, or any natural or artificially designed protein or scaffold.

For use in vivo, polyoxyalkelene moieties may be attached to the peptides to enhance bioavailability or promote solubility. In preferred embodiments the polyoxyalkelene moiety is polyethelene glycol (PEG) and between 1 and 10000 PEG molecules are attached to the peptide.

In certain embodiments, the binding peptides of the invention may further comprise post-translational modifications. Exemplary post-translational protein modification include phosphorylation, acetylation, methylation, ADP-ribosylation, ubiquitination, glycosylation, carbonylation, sumoylation, biotinylation or addition of a polypeptide side chain or of a hydrophobic group. As a result, the modified soluble peptides may contain non-amino acid elements, such as lipids, poly- or mono-saccharide, and phosphates. A preferred form of glycosylation is sialylation, which conjugates one or more sialic acid moieties to the peptide or mimetic thereof of the invention. Sialic acid moieties improve solubility and serum half-life while also reducing the possible immunogeneticity of the protein. See, e.g., Raju et al. Biochemistry. 2001 Jul. 31; 40 (30): 8868-76. Effects of such non-amino acid elements on the functionality of a peptide may be tested for its antagonizing role in VEGF or VEGFR function, e.g., its inhibitory effect on angiogenesis or on tumor growth.

In one specific embodiment of the present invention, modified forms of the subject soluble peptides comprise linking the subject soluble polypeptides to nonproteinaceous polymers. In one specific embodiment, the polymer is polyethylene glycol (“PEG”), polypropylene glycol, or polyoxyalkylenes, in the manner as set forth in U.S. Pat. No. 4,640,835; 4,496,689; 4,301,144; 4,670,417; 4,791,192; or 4,179,337, which are incorporated herein by reference.

PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented by the formula: X—O—(CH₂—CH₂—O—)ₙ—H where n is 20 to 10000 and X is H or a terminal modification, e.g., a C₁₄ alkyl. In some embodiments, PEG terminates on one end with hydroxy or methoxy, i.e., X is H or CH3 (“methoxy PEG”). A PEG can contain further chemical groups which are necessary for binding reactions; which results from the chemical synthesis of the molecule; or which is a spacer for optimal distance of parts of the molecule. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEG are described in, for example, EP-A 0473 084 and U.S. Pat. No. 5,932,462, which are incorporated herein by reference. One form of PEGs includes two PEG side-chains (PEG2) linked via the primary amino groups of a lysine (Monfardini, C., et al., Bioconjugate Chem. 6 (1995) 62-69). PEG may be attached by site-directed pegylation, e.g., by conjugation of PEG to a cysteine moiety at the N- or C-terminus. A PEG moiety may also be attached by other chemistry, including by conjugation to amines

PEG conjugation to peptides or proteins generally involves the activation of PEG and coupling of the activated PEG-intermediates directly to target proteins/peptides or to a linker, which is subsequently activated and coupled to target proteins/peptides (see Abuchowski, A. et al, J. Biol. Chem., 252, 3571 (1977) and J. Biol. Chem., 252, 3582 (1977), Zalipsky, et al., and Harris et. al., in: Poly (ethylene glycol) Chemistry: Biotechnical and Biomedical Applications; (J. M. Harris ed.) Plenum Press: New York, 1992; Chap. 21 and 22; and U.S. patent application Ser. No. 11/890,162 which is incorporated herein by reference). It is noted that a binding peptide containing a PEG molecule is also known as a conjugated protein, whereas the protein lacking an attached PEG molecule can be referred to as unconjugated.

A variety of molecular mass forms of PEG can be selected, e.g., such that the number of individual PEG monomers is between 1 and 10000, for conjugating to VEGF binding peptides. It is preferred that the combined molecular mass of PEG on an activated linker is suitable for pharmaceutical use.

One skilled in the art can select a suitable molecular mass for PEG, e.g., based on how the pegylated binding polypeptide will be used therapeutically, the desired dosage, circulation time, resistance to proteolysis, immunogenicity, and other considerations. For a discussion of PEG and its use to enhance the properties of proteins, see N. V. Katre, Advanced Drug Delivery Reviews 10: 91-114 (1993).

In one embodiment of the invention, PEG molecules may be activated to react with amino groups on a binding peptide or mimetic thereof of the invention, such as with lysines (Bencham C. O. et al., Anal. Biochem., 131, 25 (1983); Veronese, F. M. et al., Appl. Biochem., 11, 141 (1985); Zalipsky, S. et al., Polymeric Drugs and Drug Delivery Systems, adrs 9-110 ACS Symposium Series 469 (1999); Zalipsky, S. et al., Europ. Polym. J., 19, 1177-1183 (1983); Delgado, C. et al., Biotechnology and Applied Biochemistry, 12, 119-128 (1990), which are all incorporated herein by reference).

In one specific embodiment, carbonate esters of PEG are used to form the PEG-binding polypeptide conjugates. N,N′-disuccinimidylcarbonate (DSC) may be used in the reaction with PEG to form active mixed PEG-succinimidyl carbonate that may be subsequently reacted with a nucleophilic group of a linker or an amino group of a binding peptide (see U.S. Pat. No. 5,281,698 and U.S. Pat. No. 5,932,462 which are incorporated herein by reference). In a similar type of reaction, 1,1′-(dibenzotriazolyl) carbonate and di-(2-pyridyl) carbonate may be reacted with PEG to form PEG-benzotriazolyl and PEG-pyridyl mixed carbonate (U.S. Pat. No. 5,382,657, incorporated herein by reference), respectively.

Pegylation of peptides of the invention can be performed according to the methods of the state of the art, for example by reaction of the binding polypeptide with electrophilically active PEGs (supplier: Shearwater Corp., USA, shearwatercorp.com). Preferred PEG reagents of the present invention are, e.g., N-hydroxysuccinimidyl propionates (PEG-SPA), butanoates (PEG-SBA), PEG-succinimidyl propionate or branched N-hydroxysuccinimides such as mPEG2-NHS (Monfardini, C., et al., Bioconjugate Chem. 6 (1995) 62-69). Such methods may used to pegylated at an s-amino group of a binding polypeptide lysine or the N-terminal amino group of the binding polypeptide.

In another embodiment, PEG molecules may be coupled to sulfhydryl groups on a binding polypeptide (Sartore, L., et al., Appl. Biochem. Biotechnol., 27, 45 (1991); Morpurgo et al., Biocon. Chem., 7, 363-368 (1996); Goodson et al., Bio/Technology (1990) 8, 343; U.S. Pat. No. 5,766,897). U.S. Pat. Nos. 6,610,281 and 5,766,897, which are incorporated herein by reference, describe exemplary reactive PEG species that may be coupled to sulfhydryl groups.

In some embodiments, PEG molecules are conjugated to cysteine residues on a binding peptide. Mutations may be introduced into a binding peptide coding sequence to generate cysteine residues. This might be achieved, for example, by mutating one or more amino acid residues to cysteine. Preferred amino acids for mutating to a cysteine residue include serine, threonine, alanine and other hydrophilic residues.

In some embodiments, the pegylated binding peptide or mimetic thereof of the invention comprises a PEG molecule covalently attached to the alpha amino group of the N-terminal amino acid. Site specific N-terminal reductive amination is described in Pepinsky et al., (2001) JPET, 297, 1059, and U.S. Pat. No. 5,824,784, which are incorporated herein by reference. The use of a PEG-aldehyde for the reductive amination of a protein utilizing other available nucleophilic amino groups is described in U.S. Pat. No. 4,002,531, in Wieder et al., (1979) J. Biol. Chem. 254, 12579, and in Chamow et al., (1994) Bioconjugate Chem. 5, 133, which are all incorporated herein by reference.

In another embodiment, pegylated binding polypeptide comprises one or more PEG molecules covalently attached to a linker, which in turn is attached to the alpha amino group of the amino acid residue at the N-terminus of the binding polypeptide. Such an approach is disclosed in U.S. Patent Publication No. 2002/0044921 and in WO94/01451, which are incorporated herein by reference.

In one embodiment, a binding polypeptide is pegylated at the C-terminus. In a specific embodiment, a protein is pegylated at the C-terminus by the introduction of C-terminal azido-methionine and the subsequent conjugation of a methyl-PEG-triarylphosphine compound via the Staudinger reaction. This C-terminal conjugation method is described in Cazalis et al., C-Terminal Site-Specific PEGylation of a Truncated Thrombomodulin Mutant with Retention of Full Bioactivity, Bioconjug Chem. 2004; 15 (5): 1005-1009.

Monopegylation of a binding peptide or mimetic thereof of the invention can also be produced according to the general methods described in WO 94/01451, which is incorporated herein by reference. WO 94/01451 describes a method for preparing a recombinant polypeptide with a modified terminal amino acid alpha-carbon reactive group.

The ratio of a binding polypeptide to activated PEG in the conjugation reaction can be from about 1:0.5 to 1:50, between from about 1:1 to 1:30, or from about 1:5 to 1:15. Various aqueous buffers can be used in the present method to catalyze the covalent addition of PEG to the binding peptide or mimetic thereof of the invention. In one embodiment, the pH of a buffer used is from about 7.0 to 9.0. In another embodiment, the pH is in a slightly basic range, e.g., from about 7.5 to 8.5. Buffers having a pKa close to neutral pH range may be used, e.g., phosphate buffer.

Conventional separation and purification techniques known in the art can be used to purify PEGylated binding peptide or mimetic thereof of the invention, such as size exclusion (e.g. gel filtration) and ion exchange chromatography. Products may also be separated using SDS-PAGE. Products that may be separated include mono-, di-, tri-poly- and un-pegylated binding polypeptide, as well as free PEG

In one embodiment, PEGylated binding peptides or mimetics thereof of the invention contain one, two or more PEG moieties. In one embodiment, the PEG moiety(ies) are bound to an amino acid residue which is away from the surface that contacts the target ligand. In some embodiments, the PEG in pegylated binding polypeptide is a substantially linear, straight-chain PEG

In another embodiment, the pegylated binding polypeptides of the invention will preferably retain at least 25%, 50%, 60%, 70% least 80%, 85%, 90%, 95% or 100% of the biological activity associated with the unmodified protein. In one embodiment, biological activity refers to its ability to bind to VEGF, as assessed by KD, kₒₙ or k_{off}. In one specific embodiment, the pegylated binding polypeptide protein shows an increase in binding to VEGF relative to unpegylated binding polypeptide.

The serum clearance rate of PEG-modified polypeptide may be decreased by about 10%, 20%, 30%, 40%, 50%, 60%, 70%, 80%, or even 90%, relative to the clearance rate of the unmodified binding polypeptide. The PEG-modified polypeptide may have a half-life (t_{1/2}) which is enhanced relative to the half-life of the unmodified protein. The half-life of PEG-binding polypeptide may be enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of the unmodified binding polypeptide. In some embodiments, the protein half-life is determined in vitro, such as in a buffered saline solution or in serum. In other embodiments, the protein half-life is an in vivo half life, such as the half-life of the protein in the serum or other bodily fluid of an animal.

### IV. Assays for Identifying Peptides of the Invention

A VEGF binding peptide or mimetic thereof of the present invention may be tested to determine whether it is effective in antagonizing VEGF or VEGF biological activity. One method of testing the peptide or mimetic thereof is to confirm that interaction occurs between the peptide and VEGF. Several preferred assays are described herein, particularly in Examples 5, 7, and 12. Tests for binding are well known in the art and may include labeling (e.g., radiolabeling) the peptide, incubating the peptide with VEGF under conditions in which binding may occur, and then isolating/visualizing the complex on a gel or phosphor screen. The ELISA technique may be employed to determine binding. In one embodiment, VEGF may be coated onto ELISA plate wells. Biotin-tagged peptides of the invention are then added. After washing, binding of peptides to VEGF may be determined by treatment with streptavidin/HRP cognate which allows colorimetric detection. In another embodiment, biotin tagged peptides are immobilized onto streptavidin coated magnetic beads. Said bead-bound peptides are then treated with VEGF. After washing, the beads are subjected to denaturing conditions sufficient to release any bound VEGF which is then detected, for example, by silver-stained SDS-PAGE gel.

Immunoassays may be employed in receptor internalization studies, receptor activation studies, receptor detection assays, or assays designed to measure the binding of VEGF to a peptide of the invention. Labeling agents may be attached to peptide or mimetic thereof of the invention or to VEGF, as required by the experiment. The labeling agent can be modified with a detectable agent, such as biotin, to which another molecule can specifically bind, such as streptavidin. A variety of detectable moieties are well known to those skilled in the art.

Commonly used assays include noncompetitive assays, e.g., sandwich assays, and competitive assays. Commonly used assay formats include Western blots (immunoblots), which are used to detect and quantify the presence of protein in a sample. The particular label or detectable group used in the assay is not a critical aspect of the invention, as long as it does not significantly interfere with the specific binding of the immunoglobulin used to detect the target molecule (e.g., VEGF, or a VEGF receptor) or a peptide of the invention which is designed to bind VEGF. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene or latex). The label may be coupled directly or indirectly to the desired component of the assay according to methods well known in the art. The label can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, and the like. Chemiluminescent compounds include luciferin, and 2,3-dihydrophthalazinediones, e.g., luminol. For a review of various labeling or signal producing systems that may be used, see U.S. Pat. No. 4,391,904, which is incorporated herein by reference.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Binding of a peptide or mimetic thereof of the invention to VEGF may also be determined by Fluorescence Resonance Energy Transfer (FRET) analysis. A comprehensive review of fluorescence methodologies for determining protein conformations and interactions can be found in Johnson (2005) Traffic. 2005 December; 6(12):1078-92 which is incorporated herein by reference. In the FRET assay VEGF and the peptide or mimetic thereof of the invention are labeled with appropriate FRET fluorophores. Labeled VEGF is then incubated with test peptides of the invention. FRET analysis will allow the observation of binding interactions between VEGF and the peptides of the invention. There are a number of methods available to perform FRET analysis, and a large portion of the variation arises from the use of different fluorophores or different techniques to incorporate those fluorophores into proteins of interest. FRET fluorophores and analysis methods are well known in the art, and a brief review of FRET technology is available in Heyduk. (2002) Current Opinion in Biotechnology. 13(4). 292-296 and references therein. The following publications expand on the FRET method and are incorporated herein by reference: Kajihara et al. (2006) Nat. Methods. 3(11):923-9; Biener-Ramanujan et al. (2006) Growth Horm IGF Res. 16(4):247-57; Taniguchi et al. (2007) Biochemistry. 46(18):5349-57; U.S. Pat. Nos. 6,689,574; 5,891,646; and WIPO Publication No. WO/2002/033102. FRET fluorophores may be incorporated into any region of VEGF or the peptides of the invention to detect conformational changes or binding interactions provided that the fluorophores do not substantially interfere with the native conformation of VEGF or the ability of peptides of the invention to bind VEGF.

Fluorophores useful for FRET are often the same as those useful for Bioluminescence Resonance Energy Transfer (BRET) as discussed below. Additional methods and useful fluorophores for FRET are described in Neininger et al. (2001) EMBO Reports. 2(8):703-708; Huebsch and Mooney (2007) Biomaterials. 28(15):2424-37; Schmid and Birbach (2007) Thromb Haemost. 97(3):378-84; Jares-Erijman AND Jovin (2006) Curr Opin Chem. Biol. 10(5):409-16; Johansson (2006) Methods Mol. Biol. 335:17-29; Wallrabe and Periasamy (2005) Curr Opin Biotechnol. 16(1):19-27; and Clegg R M (1995) Curr Opin Biotechnol. 6(1):103-10 which are incorporated herein by reference.

As discussed in U.S. Pat. Pub. No. 20060199226 which is incorporated herein by reference, BRET based assays can be used to monitor the interaction of proteins having a bioluminescent donor molecule (DM) with proteins having a fluorescent acceptor moiety (AM). Briefly, a VEGF-DM fusion will convert the substrate's chemical energy into light. If there is an AM (e.g., a peptide-AM fusion) in close proximity to the VEGF-DM fusion, the binding interaction will emit light at a certain wavelength. For example, BRET based assays can be used to assess the interaction between a VEGF-luciferase fusion and a GFP-peptide fusion. This differs slightly from FRET analysis, where the donor molecule may be excited by light of a specific wavelength rather than by chemical energy conversion. Examples of bioluminescent proteins with luciferase activity that may be used in a BRET analysis may be found in U.S. Pat. Nos. 5,229,285, 5,219,737, 5,843,746, 5,196,524, 5,670,356, which are incorporated herein by reference. Alternative DMs include enzymes, which can act on suitable substrates to generate a luminescent signal. Specific examples of such enzymes are beta-galactosidase, alkaline phosphatase, beta-glucuronidase and beta-glucosidase. Synthetic luminescent substrates for these enzymes are well known in the art and are commercially available from companies, such as Tropix Inc. (Bedford, Mass., USA). DMs can also be isolated or engineered from insects (U.S. Pat. No. 5,670,356, which is incorporated herein by reference).

Depending on the substrate, DMs emit light at different wavelengths. Non-limiting examples of substrates for DMs include coelenterazine, benzothiazole, luciferin, enol formate, terpene, and aldehyde, and the like. The DM moiety can be fused to either the amino terminal or carboxyl terminal portion of the VEGF protein. AMs in BRET analysis may re-emit the transferred energy as fluorescence. Examples of AMs include Green Fluorescent Protein (GFP), or isoforms and derivatives thereof such as YFP, EGFP, EYFP and the like (R. Y Tsien, (1998) Ann Rev. Biochem. 63:509-544). Preferably, the positioning of the AM domain within the AM-peptide fusion does not alter the activity peptide. In other embodiments the BRET analysis may be used to determine VEGF-receptor dimerization or activation

In preferred embodiments biosensor experiments using, for example, a BIACORE instrument, may be carried out to determine binding of VEGF to the peptides or mimetics thereof of the invention. In such an embodiment, peptides of the invention are immobilized on to a streptavidin coated chip. VEGF containing solutions are then flowed over the chip surface using the BIACORE microfluidics system, and binding events are detected by measuring the surface plasmon resonance effect.

In addition, in vivo models may be used. For example, a key parameter in determining whether a D-peptide will be potentially effective in an oncology or macular degeneration indication will be its ability to block the formation of new blood vessels in response to VEGF stimulation. While receptor ELISA and HUVEC cell growth assays are useful indicators of whether D-peptides can act on or inhibit the appropriate receptors, they do not directly measure the ability to stop angiogenesis. The Corneal Angiogenesis model is an established model in which researchers surgically place a VEGF-releasing implant into a rat cornea. The cornea is naturally avascular, so growth of blood vessels in response to the VEGF release can be observed by visual inspection. This assay has the advantage of speed, as angiogenesis resulting from the VEGF-releasing implant can be quantified after only 3-5 days. An animal with the corneal implant treated with a saline infusion may be used as a negative control. A commercially available anti-VEGF therapeutic is selected (e.g., Avastin or Macugen) and administered as a positive control. Test animals receive selected D-peptides systemically in an IV dosage.

Another rodent model, intraocular implant model, is available in which a gel pellet impregnated with VEGF is intraocularly implanted, causing an AMD-like condition. In this model, drugs to be tested are delivered by intraocular injection, more closely simulating the conditions under which the D-peptides are likely to be used in a clinical setting. This test requires the growth of blood vessels in the retina to be evaluated and therefore is more expensive than the corneal angiogenesis model. However, this model has better predictive value for potential efficacy in a clinical setting compared to the corneal model.

Animal models of choroidal neovascularization (CNV) may also be used to test the efficacy of the peptides or mimetics thereof of the invention. Such CNV models are used as a laboratory model of Acute Macular Degeneration. Visual loss develops in the exudative form of AMD because abnormal choroidal neovascular membranes (CNVMs) develop under the retina, leak serous fluid and blood, and ultimately cause a blinding disciform scar in and under the retina. CNV primate and rat models are available which may be used to test the peptides of the invention. In a preferred embodiment the choroidal neovascular process is initiated by art recognized methods, e.g., subretinal implantation of growth factor impregnated pellets or, more preferably, traumatic laser injury. In such models, the inhibition, prevention, or reduction of neovascularization by a peptide or mimetic thereof of the invention indicates that the peptide was successful reducing angiogenesis.

Various methods and models are known in the art to measure angiogenesis and VEGF biological activity which may be used to test peptides of the present invention. As a further example, the ability of the peptides to specifically inhibit angiogenesis and growth of tumor cells may be assessed by examining their effect on proliferation of MCF-7 cells, MCF-7 cells transfected with VEGF, or other cell lines. In addition, sprouting angiogenesis may be measured according to methods known in the art (Issbrücker et al. FASEB J. 2003 February; 17(2):262-4 and Rajashekhar et al. J Vasc Res. 2006; 43(2):193-204 which are both incorporated herein by reference). Such an analysis may be performed, for example, by obtaining human microvascular endothelial cells and growing them to confluency on collagen coated microcarriers (e.g., beads). The microcarriers may then be suspended in fibrinogen and polymerization will be initiated by the addition of thrombin. After a period of incubation (e.g., 24 hrs) sprouts may be counted, e.g., as described in Issbrücker et al. FASEB J. 2003 February; 17(2):262-4 or Rajashekhar et al. J Vasc Res. 2006; 43(2):193-204, incorporated herein by reference.

The peptides or mimetics thereof of the invention may be screened to determine whether they inhibit VEGF biological activity by antagonizing VEGF receptor activity by using assays described herein and those assays that are well known in the art. For example, assays which may determine receptor internalization, receptor autophosphorylation, and/or kinase signaling may be used to identify peptides which prevent the activation of a VEGF receptor, e.g., VEGFR-2. As described in Example 5, a HUVEC cell-based assay may be used to determine the ability of a peptide or mimetic thereof of the invention to inhibit VEGF biological activity. In such an assay, cells treated with VEGF respond by proliferating. In some embodiments a peptide of the invention will block such VEGF induced proliferation.

Screening for new inhibitor peptides may be accomplished by using standard methods known in the art, for example, by employing a phosphoELISA™ procedure (available at Invitrogen) to determine the phosphorylation state of the VEGF receptor or a downstream molecule. The phosphorylation state of the receptor may be determined using commercially available kits such as, for example, VEGFR2 [pY949] PAb hu, ms (Invitrogen, SKU# 44-1041G). Peptides and mimetics thereof of the invention may be screened using such kits to determine their VEGF receptor inhibitory activity. For example, after treatment with VEGF and a peptide of the invention, a phosphoELISA™ may be performed to determine the phosphorylation state and, thus, the activation state of a VEGF receptor of interest. Peptides of further interest could be identified as those which prevent receptor activation. Other methods to detect phosphorylation events include those described in U.S. Pat. No. 6,548,266; or Goshe et al. (2006) Brief Funct Genomic Proteomic. 4:363-76; de Graauw et al. (2006) Electrophoresis. 27:2676-86; Schmidt et al. (2007) J Chromatogr B Analyt Technol Biomed Life Sci. 849:154-62; or by the use of the FlashPlates (SMP200) protocol for the Kinase Phosphorylation Assay using [gamma-33P]ATP by PerkinElmer, all of which are incorporated herein by reference. Other immunoassays, such a simple ELISA, which employ a labeling agent to specifically bind to and label the complex formed by a detecting antibody and a receptor of interest (see U.S. Pat. No. 7,056,685 which is incorporated herein by reference). The labeling agent may itself be the antibody used to detect a receptor. Alternatively, the labeling agent may be a third agent, such as a secondary or tertiary antibody (e.g., and anti-mouse antibody binding to mouse monoclonal antibody specific for the VEGF receptor). Such immunoassays (using labels and reagents described above) may be used to detect phosphorylated (i.e., activated) receptor, or phosphorylated second messengers. In some preferred embodiments, successful peptides of the invention are those which bind VEGF and prevent the subsequent activation of the VEGF receptor.

A receptor ELISA assay may also be used to detect the ability of peptides of the invention to inhibit VEGF binding to VEGF receptor. Such assays are described herein, at least in Example 5 below.

Since receptor activation may lead to endocytosis and receptor internalization, it is useful, in some embodiments, to determine the ability of the peptides and mimetics thereof of the invention to inhibit VEGF receptors by measuring their ability to prevent receptor internalization. Receptor internalization assays are well known in the art and described in, for example, Fukunaga et al. (2006) Life Sciences. 80(1). p. 17-23; Bernhagen et al. (2007) Nature Medicine 13, 587-596; natureprotocols.com/2007/04/18/receptor_internalization_assay.php), the entire contents of each of which are incorporated herein by reference. One well-known method to determine receptor internalization is to tag a ligand with a fluorescent protein, e.g., Green Fluorescent Protein (GFP), or other suitable labeling agent. Upon binding of the ligand to the receptor, fluorescence microscopy may be used to visualize receptor internalization. Similarly, a VEGF receptor (or VEGF or a peptide or mimetic thereof of the invention) may be tagged with a labeling agent and fluorescence microscopy may be used to visualize receptor internalization. If the peptide or mimetic thereof of the invention is able to bind VEGF, thereby preventing VEGF binding to and activating its receptor, lessened internalization of fluorescence will be observed in the presence of VEGF and peptide as compared to appropriate controls (e.g., fluorescence may be observed only at the periphery of the cell where VEGF binds the receptor rather than in endosomes or vesicles).

In addition to those mentioned above, various other receptor activation assays are known in the art, any of which may be used to evaluate the function of the moieties of the invention. Further receptor activation assays which may be used in accordance with the present invention are described in U.S. Pat. Nos. 6,287,784; 6,025,145; 5,599,681; 5,766,863; 5,891,650; 5,914,237; 7,056,685; and many scientific publications including, but not limited to: Amir-Zaltsman et al. (2000) Luminescence 15(6):377-80; Nakayama and Parandoosh (1999) Journal of Immunological Methods. 225(1-2), 27, 67-74; Pike et al. (1987) Methods of Enzymology 146: 353-362; Atienza et al. (2005) Journal of Biomolecular Screening. 11(6): 634-643; Hunter et al. (1982). Journal of Biological Chemistry 257(9): 4843-4848; White and Backer (1991) Methods in Enzymology 201: 65-67; Madden et al. (1991) Anal Biochem 199: 210-215; Cleaveland et al. (1990) Analytical Biochemistry 190: 249-253; Lazaro et al. (1991) Analytical Biochemistry 192: 257-261; Hunter and Cooper (1985) Ann Rev Biochem 54: 897-930; Ullrich and Schlessinger (1990) Cell 61: 203-212; Knutson and Buck (1991) Archives of Biochemistry and Biophysics 285(2): 197-204); King et al. (1993) Life Sciences 53: 1465-1472; Wang. (1985) Molecular and Cellular Biology 5(12): 3640-3643; Glenney et al. (1988) Journal of Immunological Methods 109: 277-285; Kamps (1991) Methods in Enzymology 201: 101-110; Kozma et al. (1991) Methods in Enzymology 201: 28-43; Holmes et al. (1992) Science 256: 1205-10; and Corfas et al. (1993) PNAS, USA 90: 1624-1628, all of which are incorporated herein by reference.

Receptor activation by ligand binding typically initiates subsequent intracellular events, e.g., increases in secondary messengers such as IP₃ which, in turn, releases intracellular stores of calcium ions. Thus, receptor activity may be determined by measuring the quantity of secondary messengers such as IP₃, cyclic nucleotides, intracellular calcium, phosphorylated signaling molecules, or other possible targets known in the art. U.S. Pat. No. 7,056,685 describes and references several methods which may be used in accordance with the present invention to detect receptor activity and is incorporated herein by reference.

Many of the assays described above, such as receptor internalization assays or receptor activation assays may involve the detection or quantification of the VEGF receptor using immunological binding assays (e.g., when using a radiolabeled antibody to detecting the amount of VEGF or VEGF receptor on the cell surface during a receptor internalization assay). Immunological binding assays are widely described in the art (see, e.g., U.S. Pat. Nos. 4,366,241; 4,376,110; 4,517,288; and 4,837,168, which are incorporated herein by reference). For a review of the general immunoassays, see also Methods in Cell Biology: Antibodies in Cell Biology, volume 37 (Asai, ed. 1993); Basic and Clinical Immunology (Stites & Ten, eds., 7th ed. 1991).

Furthermore, one may use traditional cross-linking studies (exemplified by Rodriguez et al. (1990) Molecular Endocrinology, 4(12), 1782-1790) to detect VEGF receptor dimerization. In such a system, e.g., VEGF and a peptide of the invention would be incubated with a VEGF receptor. Peptides or mimetics thereof of the invention which are able to prevent binding of VEGF to the VEGF receptor would also prevent VEGF receptor dimerization. FRET and similar systems may also be used to directly measure dimerization. For example, by incorporating appropriate FRET fluorophores into the cytoplasmic domain of the VEGF receptor and into a phosphorylation target protein (i.e., a downstream signaling molecule), FRET would be capable of determining whether downstream signaling molecules were being recruited to said receptor. Therefore, in one embodiment, a successful peptide of the invention is one which prevents receptor activation, detected as lack of fluorescence by FRET or BRET (Pfleger et al. (2006) Nature Protocols 1 337-345; Kroeger et al. (2001), J. Biol. Chem., 276(16):12736-43; and Harikumar, et al. (2004) Mol Pharmacol 65:28-35; which are all incorporated herein by reference) analysis or by other receptor activation assays (e.g., autophosphorylation assay employing anti-phosphotyrosine antibodies and Western Blot). Similarly, FRET or BRET methods as described above may be used to determine the binding of VEGF to VEGF-receptor. In such embodiments, the successful peptides of the invention are those which reduce the binding of VEGF to VEGF-receptor.

Thus, using the techniques described herein, one of skill in the art can easily test peptides to determine whether they bind VEGF and/or inhibit VEGF receptor activity or whether they prevent VEGF receptor dimerization.

### V. Library Screening and Affinity Maturation

In some embodiments, peptide or gene libraries may be screened to identify potential peptides or mimetics thereof of the invention. In related embodiments, a particular peptide or mimetic thereof of the invention may be altered by affinity maturation or mutagenesis, thereby producing a library of related peptides or nucleic acids. As such, one aspect of the invention may involve screening large libraries in order to identify potential peptides or mimetics thereof (or nucleic acids encoding said peptides) of the invention. Any methods for library generation and target selection known in the art or described herein (e.g., the methods described in the Examples or in Section IV above) may be used in accordance with the present invention.

In a preferred embodiment an mRNA display library is screened to identify peptides of the invention. A preferred mRNA display methodology is described in Examples 9-11. It should be noted that the selection methodology may be carried out under conditions such that intramolecular disulfide bonds are present in the peptides or mimetics thereof of the invention during selections. In other embodiments, the formation of disulfide bonds may be prevented, if desired. Briefly, a starting library is obtained by, e.g., direct DNA synthesis or through in-vitro or in-vivo mutagenesis. The double stranded DNA library is then transcribed in-vitro and attached to a puromycin-like linker. in vitro translation is carried out wherein the puromycin-like linker reacts with the nascent translation product. The result, after purification, is a library of peptide-RNA fusion molecules. Reverse transcription generates a cDNA/RNA hybrid, covalently linked to the transcribed peptide. This complex is then selected for by using the target molecule, e.g., D-VEGF. Peptides that bind D-VEGF will be selected, and the cDNA is easily eluted to identify the selected peptides. The selection may be performed multiple times to identify higher affinity binders, and may further be implemented with competitive binders or more stringent washing conditions. One of skill in the art will appreciate that variants of the mRNA display procedure described herein may be employed to select for peptides of the invention. Indeed, the skilled artisan will appreciate that a variety of library generation and screening methodologies may be employed to identify peptides of the invention, for example, mRNA display, ribosome display, phage display, bio-panning, cell-surface display, gene-shuffling libraries, mutagenesis libraries, and the methodologies described in the articles Valencia et al. Biotechnol Prog. 2008 May-June; 24(3):561-9; Austin R J, Ja W W, Roberts R W. J Mol Biol. 2008 Apr. 11; 377(5):1406-18; Horisawa K, Doi N, Yanagawa H. PLoS ONE. 2008 Feb. 20; 3(2):e1646; Keefe A D. Curr Protoc Mol Biol. 2001 May; Chapter 24:Unit 24.5.PMID: 18265212; and Huang B C, Liu R. Biochemistry. 2007 Sep. 4; 46(35):10102-12, U.S. patent application Ser. Nos. 11/813,199; 10/477,373; 11/018,798; 11/362,309; 10/362,264; 10/855,668; 9/876,235; 11/258,833; 10/482,382; 10/196,473; 10/764,799, and U.S. Pat. Nos. 5,599,672; 7,074,557; 6,348,315; 7,270,969; 6,300,065; 6,686,168; 5,830,721; 6,423,538; 5,834,318; 5,750,344; 6,696,251; 5,866,344; 6,620,587; 5,348,867; 5,403,484; 5,427,908; 5,270,170; 5,843,701; 6,291,158; 5,338,665; 6,368,861; 6,319,714; 6,376,246; 5,605,793; 5,837,500; 5,821,047; 6,194,183; 5,702,892; 6,057,098; 6,329,209; 6,323,004; 5,766,905; 6,207,446; 6,261,804; 6,258,558; 6,066,452; and 7,270,969, which are all incorporated herein by reference.

For convenience, the selection methods described herein will be directed to discovering L-Peptides which bind D-VEGF. Subsequently, the D-peptides of the invention may be synthesized according to the selected L-peptide sequence. By symmetry, the synthesized D-peptides will bind to the native, L-form of VEGF.

### VI. Pharmaceutical Compositions Containing the Peptides or Mimetics Thereof of the Invention

In another aspect, the present invention provides a composition, e.g., a pharmaceutical composition, containing one or a combination of the peptides (e.g., two or more different peptides) of the invention, formulated together with a pharmaceutically acceptable carrier. Pharmaceutical compositions of the invention also can be administered in combination therapy, i.e., combined with other agents. For example, the combination therapy can include a peptide or mimetic thereof of the invention combined with an anti-VEGF antibody (or small molecule or peptidic molecule). Examples of therapeutic agents that can be used in a combination therapy are described in greater detail below.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., the peptide or mimetic thereof of the invention may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

In a particular embodiment the peptides or mimetics thereof of the invention may be dissolved in water with sodium chloride to achieve physiological isotonic salt conditions.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

The solid dosage forms of tablets, dragees, capsules, pills, and granules can be prepared with coatings and shells such as enteric coatings and other coatings well-known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. The peptides can also be in micro-encapsulated form, if appropriate, with one or more excipients.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

A composition formulated as a solution may be made suitable for administration by dropper into the eye, e.g., by preparing the solution to contain the appropriate amount of salts.

Liposomes containing the peptide or mimetic thereof of the present invention can be prepared in accordance with any of the well known methods such as described by Epstein et al. (Proc. Natl. Acad. Sci. USA 82: 3688-3692 (1985)), Hwang et al. (Proc. Natl. Acad. Sci. USA 77: 4030-4034 (1980)), EP 52,322, EP 36,676; EP 88,046; EP 143,949; EP 142,641; Japanese Pat. Appl. 83-118008, and EP 102,324, as well as U.S. Pat. Nos. 4,485,045 and 4,544,545, the contents of which are hereby incorporated by reference in their entirety. Liposomes may be small (about 200-800 Angstroms) unilamellar type in which the lipid content is greater than about 10 mol. percent cholesterol, preferably in a range of 10 to 40 mol. percent cholesterol, the selected proportion being adjusted for optimal peptide therapy. However, as will be understood by those of skill in the art upon reading this disclosure, phospholipid vesicles other than liposomes can also be used.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the peptide or mimetic thereof of the invention, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for a moiety of the invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

Alternatively, the peptide or mimetic thereof can be administered as a sustained release formulation, in which case less frequent administration is required. Dosage and frequency vary depending on the half-life of the administered substance in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients and small molecules in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of a peptide or mimetic thereof of the invention preferably results in a decrease in severity of disease symptoms, an increase in frequency and duration of disease symptom-free periods, or a prevention of impairment or disability due to the disease affliction. For example, for the treatment of tumors, a “therapeutically effective dosage” preferably inhibits cell growth or tumor growth by at least about 10% or 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound (e.g., a peptide or mimetic thereof of the invention) to inhibit, such inhibition in vitro by assays known to the skilled practitioner. A therapeutically effective amount of a therapeutic compound (e.g., a peptide or mimetic thereof of the invention) can decrease tumor size, or otherwise ameliorate symptoms in a subject. One of ordinary skill in the art would be able to determine such amounts based on such factors as the subject's size, the severity of the subject's symptoms, and the particular composition or route of administration selected. Methods to determine whether the peptide or mimetic thereof of the present invention is effective in antagonizing VEGF or VEGF biological activity are discussed in Section IV of this specification and in the Example section.

A composition of the present invention can be administered via one or more routes of administration using one or more of a variety of methods known in the art. As will be appreciated by the skilled artisan, the route and/or mode of administration will vary depending upon the desired results. Preferred routes of administration for binding moieties of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, a peptide or mimetic thereof of the invention can be administered via a non-parenteral route, such as a topical, epidermal or mucosal route of administration, for example, intranasally, orally, vaginally, rectally, sublingually or topically.

The active compounds (e.g., a peptide or mimetic thereof of the invention) can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978, which is incorporated herein by reference.

Therapeutic compositions can be administered with medical devices known in the art. For example, in a preferred embodiment, a therapeutic composition of the invention (e.g., a peptide or mimetic thereof of the invention) can be administered with a needleless hypodermic injection device, such as the devices disclosed in U.S. Pat. No. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

On the basis of their efficacy as inhibitors of VEGF biological activity, the peptides or mimetics thereof of the invention are effective against a number of conditions associated with inappropriate angiogenesis, including but not limited to autoimmune disorders (e.g., rheumatoid arthritis, inflammatory bowel disease or psoriasis); cardiac disorders (e.g., atherosclerosis or blood vessel restenosis); retinopathies (e.g., proliferative retinopathies generally, diabetic retinopathy, age-related macular degeneration, or neovascular glaucoma, acute macular degeneration), renal disease (e.g., diabetic nephropathy, malignant nephrosclerosis, thrombotic microangiopathy syndromes; transplant rejection; inflammatory renal disease; glomerulonephritis; mesangioproliferative glomerulonephritis; haemolyticuraemic syndrome; and hypertensive nephrosclerosis); hemangioblastoma; hemangiomas; thyroid hyperplasias; tissue transplantations; chronic inflammation; Meigs's syndrome; pericardial effusion; pleural effusion; autoimmune diseases; diabetes; endometriosis; chronic asthma; undesirable fibrosis (particularly hepatic fibrosis) and cancer, as well as complications arising from cancer, such as pleural effusion and ascites. Preferably, the VEGF-binding peptides or mimetics thereof of the invention can be used for the treatment of prevention of hyperproliferative diseases or cancer and the metastatic spread of cancers. Non-limiting examples of cancers include bladder, blood, bone, brain, breast, cartilage, colon kidney, liver, lung, lymph node, nervous tissue, ovary, pancreatic, prostate, skeletal muscle, skin, spinal cord, spleen, stomach, testes, thymus, thyroid, trachea, urogenital tract, ureter, urethra, uterus, or vaginal cancer. Additional treatable conditions can be found in U.S. Pat. No. 6,524,583, incorporated herein by reference.

As described herein, VEGF modulated diseases and angiogenesis-associated diseases include, but are not limited to, angiogenesis-dependent cancer, including, for example, solid tumors, blood born tumors such as leukemias, and tumor metastases; benign tumors, for example hemangiomas, acoustic neuromas, neurofibromas, trachomas, and pyogenic granulomas; inflammatory disorders such as immune and non-immune inflammation; chronic articular rheumatism and psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; and wound granulation and wound healing; telangiectasia psoriasis scleroderma, pyogenic granuloma, cororany collaterals, ischemic limb angiogenesis, corneal diseases, rubeosis, arthritis, diabetic neovascularization, fractures, vasculogenesis, hematopoiesis.

A VEGF binding peptide can be administered alone or in combination with one or more additional therapies such as chemotherapy, radiotherapy, immunotherapy, surgical intervention, or any combination of these. Long-term therapy is equally possible as is adjuvant therapy in the context of other treatment strategies, as described above.

In certain embodiments of such methods, one or more peptides or mimetics thereof of the invention can be administered, together (simultaneously) or at different times (sequentially). In addition, peptide therapeutic agents can be administered with another type of compounds for treating cancer or for inhibiting angiogenesis.

In certain embodiments, the subject therapeutic agents of the invention (e.g., a peptide or mimetic thereof of the invention) can be used alone. Alternatively, the subject agents may be used in combination with other conventional therapeutic approaches directed to treatment or prevention of proliferative disorders (e.g., tumor). For example, such methods can be used in prophylactic cancer prevention, prevention of cancer recurrence and metastases after surgery, and as an adjuvant of other conventional cancer therapy. The present invention recognizes that the effectiveness of conventional cancer therapies (e.g., chemotherapy, radiation therapy, phototherapy, immunotherapy, and surgery) can be enhanced through the use of a subject peptide or mimetic thereof of the invention.

A wide array of conventional compounds have been shown to have anti-neoplastic activities. These compounds have been used as pharmaceutical agents in chemotherapy to shrink solid tumors, prevent metastases and further growth, or decrease the number of malignant cells in leukemic or bone marrow malignancies.

Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

When a peptide therapeutic agent of the present invention is administered in combination with another conventional anti-neoplastic agent, either concomitantly or sequentially, such therapeutic agent may be found to enhance the therapeutic effect of the anti-neoplastic agent or overcome cellular resistance to such anti-neoplastic agent. This allows decrease of dosage of an anti-neoplastic agent, thereby reducing the undesirable side effects, or restores the effectiveness of an anti-neoplastic agent in resistant cells. Pharmaceutical compounds that may be used for combinatory anti-tumor therapy include, merely to illustrate: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine. Certain chemotherapeutic anti-tumor compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (e.g., VEGF inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In certain embodiments, pharmaceutical compounds that may be used for combinatory anti-angiogenesis therapy include: (1) inhibitors of release of “angiogenic molecules,” such as bFGF (basic fibroblast growth factor); (2) neutralizers of angiogenic molecules, such as an anti-j8bFGF antibodies; and (3) inhibitors of endothelial cell response to angiogenic stimuli, including collagenase inhibitor, basement membrane turnover inhibitors, angiostatic steroids, fungal-derived angiogenesis inhibitors, platelet factor 4, thrombospondin, arthritis drugs such as D-penicillamine and gold thiomalate, vitamin D3 analogs, alpha-interferon, and the like. For additional proposed inhibitors of angiogenesis, see Blood et al., Bioch. Biophys. Acta., 1032: 89-118 (1990), Moses et al., Science, 248: 1408-1410 (1990), Ingber et al., Lab. Invest., 59: 44-51 (1988), and U.S. Pat. Nos. 5,092,885, 5,112,946, 5,192,744, 5,202,352, and 6,573,256. In addition, there are a wide variety of compounds that can be used to inhibit angiogenesis, for example, endostatin protein or derivatives, lysine binding fragments of angiostatin, melanin or melanin-promoting compounds, plasminogen fragments (e.g., Kringles 1-3 of plasminogen), tropoin subunits, antagonists of vitronectin u, 03, peptides derived from Saposin B, antibiotics or analogs (e.g., tetracycline, or neomycin), dienogest-containing compositions, compounds comprising a MetAP-2 inhibitory core coupled to a peptide, the compound EM-138, chalcone and its analogs, and naaladase inhibitors. See, for example, U.S. Pat. Nos. 6,395,718; 6,462,075; 6,465,431; 6,475,784; 6,482,802; 6,482,810; 6,500,431; 6,500,924; 6,518,298; 6,521,439; 6,525,019; 6,538,103; 6,544,758; 6,544,947; 6,548,477; 6,559,126; and 6,569,845.

In particular, the peptides or mimetics thereof of the invention may be used in combination with Macugen, or any of the following antibodies: Amevivie, Avastin, Orthoclone OKT3, Raptiva, ReoPro, Rituxan, Simulect, Synagis, Remicade, Herceptin, Mylotarg, Campath, Zevalin, Humira, Erbitux, Xolair, CeaVac, MDX-210, Mitumomab, Afelimomab, ABX-CBL, Adalimumab, and Epratuzumab.

Depending on the nature of the combinatory therapy, administration of the peptide therapeutic agents of the invention may be continued while the other therapy is being administered and/or thereafter. Administration of the peptide or mimetic thereof of the invention may be made in a single dose, or in multiple doses. In some instances, administration of the polypeptide therapeutic agents is commenced at least several days prior to the conventional therapy, while in other instances, administration is begun either immediately before or at the time of the administration of the conventional therapy.

The VEGF binding peptides described herein can also be detectably labeled and used to visualize VEGF for imaging applications or diagnostic applications. For diagnostic purposes, a peptide or mimetic thereof of the invention is preferably immobilized on a solid support. Preferred solid supports include columns (for example, affinity columns, such as agarose-based affinity columns), microchips, or beads.

In one example of a diagnostic application, a biological sample, such as serum or a tissue biopsy, from a patient suspected of having a condition characterized by inappropriate angiogenesis is contacted with a detectably labeled peptide or mimetic thereof of the invention to detect levels of VEGF. The levels of VEGF detected are then compared to levels of VEGF detected in a normal sample also contacted with the labeled peptide. An increase of at least 10%, 20%, 30%, 40%, 50%, 60%, 70%, 80%, or 90% in the levels of the VEGF may be considered a diagnostic indicator of a condition characterized by inappropriate angiogenesis.

In certain embodiments, the VEGF binding peptides of the invention are further attached to a label that is able to be detected (e.g., the label can be a radioisotope, fluorescent compound, enzyme or enzyme co-factor). The active moiety may be a radioactive agent, such as: radioactive heavy metals such as iron chelates, radioactive chelates of gadolinium or manganese, positron emitters of oxygen, nitrogen, iron, carbon, or gallium, ⁴³K, ⁵²Fe, ⁵⁷Co, ⁶⁷Cu, ⁶⁷Ga, ⁶⁸Ga, ¹²³I, ¹²⁵I, ¹³¹I, ¹³²I, or ⁹⁹Tc. A binding agent affixed to such a moiety may be used as an imaging agent and is administered in an amount effective for diagnostic use in a mammal such as a human and the localization and accumulation of the imaging agent is then detected. The localization and accumulation of the imaging agent may be detected by radioscintigraphy, nuclear magnetic resonance imaging, computed tomography or positron emission tomography. Immunoscintigraphy using VEGF binding peptides or mimetics thereof of the invention directed at VEGF may be used to detect and/or diagnose cancers and vasculature. For example, any of the binding peptides against VEGF may be labeled with ⁹⁹Technetium, or ¹²⁵Iodine and effectively used for such imaging. As will be evident to the skilled artisan, the amount of radioisotope to be administered is dependent upon the radioisotope. Those having ordinary skill in the art can readily formulate the amount of the imaging agent to be administered based upon the specific activity and energy of a given radionuclide used as the active moiety. Imaging may be of particular use in cancers since small peptides will be more easily able to penetrate the tumor, allowing superior visualization and monitoring of tumor progression.

Typically 0.1-100 millicuries per dose of imaging agent, preferably 1-10 millicuries, most often 2-5 millicuries are administered. Thus, compositions according to the present invention useful as imaging agents comprising a targeting moiety conjugated to a radioactive moiety comprise 0.1-100 millicuries, in some embodiments preferably 1-10 millicuries, in some embodiments preferably 2-5 millicuries, in some embodiments more preferably 1-5 millicuries.

As such VEGF modulated diseases and angiogenesis related diseases, e.g., macular degeneration or cancer, may be monitored using the peptides or mimetics thereof of the invention. For example, tissue samples or biopsies may be collected from a cancer patient and the amount of VEGF in the samples may be monitored to assess the progress of the disease.

The VEGF binding peptides or mimetics thereof of the present invention may, in some embodiments, bind VEGF without inhibiting VEGF biological activity. In such embodiments, VEGF, and the bound peptide of the invention, may bind to the VEGF receptor and be internalized. Accordingly the VEGF binding peptides or mimetics thereof of the present invention can be used to deliver additional therapeutic agents (including but not limited to drug compounds, chemotherapeutic compounds, and radiotherapeutic compounds) to a cell or tissue expressing VEGF receptor. In one example, a VEGF binding peptide which is fused to a chemotherapeutic agent may bind VEGF for targeted delivery of the chemotherapeutic agent to a tumor cell or tissue expressing a VEGF receptor.

The VEGF binding polypeptides of the present invention are useful in a variety of applications, including research, diagnostic and therapeutic applications. For instance, they can be used to isolate and/or purify a receptor or portions thereof, and to study receptor structure (e.g., conformation) and function.

In certain embodiments, the binding polypeptides of fragments thereof can be labeled or unlabeled for diagnostic purposes. Typically, diagnostic assays entail detecting the formation of a complex resulting from the binding of a binding peptide to VEGF or by detecting the formation of a complex resulting from peptide-bound VEGF binding to a VEGF receptor. The binding peptides or fragments can be directly labeled, similar to antibodies. A variety of labels can be employed, including, but not limited to, radionuclides, fluorescers, enzymes, enzyme substrates, enzyme cofactors, enzyme inhibitors and ligands (e.g., biotin, haptens). Numerous appropriate immunoassays are known to the skilled artisan (see, for example, U.S. Pat. Nos. 3,817,827; 3,850,752; 3,901,654; and 4,098,876 which are incorporated herein by reference). When unlabeled, the binding polypeptides can be used in assays, such as agglutination assays. Unlabeled binding polypeptides can also be used in combination with another (one or more) suitable reagent which can be used to detect the binding polypeptide, such as a labeled antibody reactive with the binding polypeptide or other suitable reagent (e.g., labeled protein A).

In certain aspects, kits for use in detecting the presence of a VEGF protein in a biological sample can also be prepared. Such kits will include an VEGF binding peptide or mimetic thereof of the invention which binds to a VEGF protein, as well as one or more ancillary reagents suitable for detecting the presence of a complex between the binding peptide and VEGF. The peptide compositions of the present invention can be provided in lyophilized form, either alone or in combination with additional antibodies specific for other epitopes.

The binding peptides or mimetics thereof, which can be labeled or unlabeled, can be included in the kits with adjunct ingredients (e.g., buffers, such as Tris, phosphate and carbonate, stabilizers, excipients, biocides and/or inert proteins, e.g., bovine serum albumin). For example, the binding peptides can be provided as a lyophilized mixture with the adjunct ingredients, or the adjunct ingredients can be separately provided for combination by the user. Generally these adjunct materials will be present in less than about 5% weight based on the amount of active binding peptide or mimetic thereof, and usually will be present in a total amount of at least about 0.001% weight based on peptide concentration. Where a second antibody capable of binding to the peptide or mimetic thereof is employed, such antibody can be provided in the kit, for instance in a separate vial or container. The second antibody, if present, is typically labeled, and can be formulated in an analogous manner with the antibody formulations described above.

The present invention also relates to a method of detecting the susceptibility of a mammal to certain diseases. To illustrate, the method may be used to detect the susceptibility of a mammal to diseases which progress based on the amount of VEGF present in serum or tissues. In this embodiment, a sample to be tested is contacted with a binding polypeptide which binds to a VEGF or portion thereof under conditions appropriate for binding thereto, wherein the sample comprises cells which express VEGF in normal individuals. The binding and/or amount of binding is detected, which indicates the susceptibility of the individual to a disease, wherein higher levels of VEGF correlate with increased susceptibility of the individual to said disease.

The present invention is further illustrated by the following examples, which should not be construed as further limiting. The contents of all figures and all references, patents and published patent applications cited throughout this application, as well as the Figures, are expressly incorporated herein by reference in their entirety.

### EXAMPLES

### Example 1

### Synthesis and Characterization of D-VEGF

VEGF-1 exists in at least four isoforms generated by splicing at the nucleic acid level with 121, 165, 189 and 206 amino acids. All of the isoforms are capable of binding to and activating VEGFR-1 and VEGFR-2, but differ in their binding to cell-surface heparin sulfates and the extracellular matrix (ECM). VEGF121 is a freely diffusible protein, while the larger isoforms appear to become immobilized by heparin and ECM binding in vivo. All VEGF-1 isoforms are homodimers covalently joined by intermolecular disulfide bonds. Furthermore, all VEGF-1 isoforms appear to share a common receptor binding cysteine-knot domain which is contained within residues 8-109. This domain has been structurally characterized by both NMR and X-ray crystallographic methods. A number of avenues to inhibition of VEGF signaling are being pursued, including decoy receptors (VEGF-Trap, Regeneron), modified nucleic acid aptamers (Macugen, Eyetech/Pfizer) antibodies directed against VEGF receptors, antisense induced downregulation of VEGF and its receptors and antiangiogenic ribozymes (Angiozyme, Ribozyme Pharmaceuticals) but the best characterized system remains direct blockade of VEGF signaling by monoclonal antibody (Avastin, Genentech).

Since all naturally occurring VEGF-1 isoforms contain the core receptor binding domain (RBD) (residues 8-109), this motif was initially chosen for synthesis. This choice streamlined the synthetic steps, since the smaller protein required fewer synthetic peptides (2) and ligation steps (1). It is also be beneficial in the peptide selection stages, since VEGF RBD lacks the heparin and ECM-affinity domains present in the larger isoforms, there is no risk of selecting inactive peptides that target these regions.

The 101-residue sequence of VEGF RBD was broken into two large peptides. The C-terminal peptide had a free acid C-terminus and an unprotected Cys residue at its amino terminus. The amino terminal peptide had a C-terminal thioester group.

TABLE 1

| | | SEQ |
|---|---|---|
| | | ID |
| Peptide | Sequence | NO |
| VEGF (8-50)-thioester | GQNHHEV VKFMDVYQRS | 32 |
| | YCHPIETLVD IFQEYPDEIE | |
| | YIFKPS | |
| VEGF (51-101)-thioester | CVPL MRCGGCCNDE | 33 |
| | GLECVPTEES NITMQIMRIK | |
| | PHQGQHIGEM | |
| | SFLQHNKCEC RPKKD | |

### Methods

A. Synthesis. Peptide building blocks for use in protein synthesis were prepared on polymer supports using solid-phase peptide synthesis (SPPS) protocols. Free acid peptides were prepared using commercially available Boc-amino acid-OCH₂-PAM DVB resins. Peptide thioesters were made on a DVB resin modified with a proprietary thioester linker. It was found that a variation of SPPS cycles based on the well-established Boc/benzyl protection strategy utilizing an in-situ neutralization procedure are an effective route to long peptides and peptide thioesters. Run on automated peptide synthesizers (ABI 433A), the SPPS cycles have enabled rapid, high yield assembly of many L-peptide sequences. First an L-VEGF was synthesized and then functionally tested, as an analogy to D-VEGF since such testing is not possible on the D-isomer version. This also allows the process could be fine tuned and optimized for production of the D-VEGF. SPPS of peptides using D-amino acids should consistently yield equivalent results to peptides made with L-amino acids. In practice, it was found that subtle differences can strongly influence product quality. This was addressed by analyzing and tightly controlling the purity of Boc-protected D-amino acids used in peptide synthesis. After completion of chain assembly by automated SPPS, the peptides were cleaved from the resin by treatment with anhydrous HF in the presence of 10% p-cresol as a scavenger (1 hr, 0° C.). Lyophilized crude peptides were then purified by HPLC. The first choice for purification of crude D-peptides was reversed phase chromatography with a C4 stationary phase and aqueous acetonitrile with 0.1% TFA as the mobile phase. Fractions from HPLC purification were analyzed by ES-MS and analytical HPLC. Fractions containing the target peptide at the desired purity were pooled and lyophilized before use in ligation reactions. Peptide ligation takes place between a peptide that has a mildly activated C-terminal thioester group and a second peptide that typically has an N-terminal Cys residue. The two peptides appear to react initially in a reversible manner by transthioesterification, followed by an irreversible rearrangement reaction that gives the amide-linked ligation product. This reaction takes place in aqueous buffer and is complete after stirring overnight at room temperature. Denaturants (guanadine hydrochloride or urea) were used to ensure solubility of the peptide segments at high concentrations (1-5 mM) to facilitate ligation. 0.5% thiophenol was added as a catalyst.

B. Folding. Following completion of the peptide ligation reactions, the ligation product was purified by RP-HPLC, and the purified material folded into the desired three-dimensional structure. There is no universal procedure for folding proteins. However, the approach applied here employed a mildly denaturing solution and a redox buffer to enable shuffling of any disulfide bonds that may be present. After finding general conditions that appeared suitable, the conditions were then optimized to maximize recovery of folded protein.

It was found that D-VEGF appears to prefer folding from redox buffered solutions with a slightly more oxidizing redox potential compared to many other proteins. It was further discovered that overnight dialysis of linear protein dissolved in 6 M guanidine hydrochloride against a solution of 0.5 M guanidine hydrochloride with 100 mM TRIS pH 8.5, 0.5 mM cysteine, 1 mM cystine 500:1 dilution) gave excellent results. Analysis of the folding reactions at short times (e.g., t=1 hr) revealed a complex mixture of species present, likely corresponding to a mixture of partially folded chains with different disulfide connectivity. After overnight dialysis, however, this mixture collapses to a single predominant peak corresponding to the properly folded protein domain. Folding yields ranged from 12-17% and the DRx team eventually obtained 10 mg of highly purified D-VEGF for characterization.

D-VEGF is a 24 kDa homodimer, larger than the synthetic interleukins our group has previously prepared (8.6 kDa). This posed significant challenges, and some difficulties were encountered in preparing the long D-peptides required for the peptide ligation approach. In early syntheses, deletion of Cys residues was observed, evident in mass spectra analysis of the purified material. This difficulty was overcome by modifying the synthetic procedures to eliminate this deletion by double coupling Cys residues.

While there has been success folding proteins in the chemokine family such as IL8, VEGF belongs to a structurally distinct class of proteins known as the cysteine knots. Severe difficulties were encountered when folding was attempted on another member of this class of proteins, TGF-b. The structure of the core cysteine knot motif is intricate, with a disulfide bond interpenetrating a larger ring formed by two other disulfide bonds. The large number of free sulfydryl groups in the synthetic protein could lead to the formation of many misfolded forms of the protein with incorrect disulfide crosslinking. It was expected that misfolded VEGF peptides would be difficult to purify out and/or detect. However, folding of VEGF was less difficult than anticipated, and optimized conditions were found more quickly than expected.

### Example 2

### Characterization of the Synthetic Peptide

Several techniques were employed to characterize the synthesized VEGF peptides. First, using Nuclear Magnetic Resonance (NMR) “Fingerprinting,” the spectrum of biologically active L-protein was directly compared with that of its enantiomer for structural confirmation without the lengthy process of assigning resonances to specific protein hydrogens. One advantage of this technique is that samples characterized by NMR are not destroyed and may still be used in screening experiments. The NMR fingerprint of synthetic L-VEGF was acquired and it was established that it is active in an in vitro cell based assay. High-field magnet time is available on a contract basis from several possible vendors, one of which was chosen for the experiments described herein. High-resolution data from NMR experiments on VEGF are available in the literature and a full assignment is available from MagRes Bank, which will facilitate NMR fingerprinting of D-VEGF. Both proteins (L- and D-VEGF) exhibited well-dispersed amide proton regions in the spectra and a single amide resonance shifted to extremely low field (>12 ppm) indicating a folded structure. A large resonance observed at approximately 3.7 ppm was due to a buffer impurity and not related to the protein signal. Spectra were collected at the 500 MHz magnet at UC Davis.

The signal intensity of the L-VEGF sample was higher than that of the D-VEGF sample, due to differences in sample concentration. However, both 1 and 2-D data indicated that both proteins were folded and moreover that they had indistinguishable folds. An analysis of the proton NOESY fingerprint region showed that the L-VEGF spectrum had 88 defined cross peaks while the D-VEGF spectrum had 74. Each of the 74 cross peaks in the D-VEGF sample had a very close chemical shift correlation to the corresponding peak in the L-VEGF sample. All of the cross peaks in the fingerprint region of the spectrum from the D-VEGF sample are therefore accounted for.

A second technique used to characterize the peptide was Mass Spectrometry. Although several different conditions were investigated, it was found that folded VEGF's do not appear to ionize efficiently in electrospray mode. The reasons for this are unclear. The unfolded protein does ionize, and can be detected by ES-MS instruments, and there is no obvious reason why the folded protein does not. In any case, the mass of the folded protein was determined by matrix-assisted laser desorption mass spectrometry (MALDI-MS). Data acquisition was carried out at M-Scan, Inc. Notwithstanding the foregoing, a molecular weight of 23,854 Da was found, in excellent agreement with the theoretical mass (23,858). A smaller peak, the double charged ion, was also observed. There is some evidence for the presence of some monomeric protein in the smaller peak, however quantitation is not available.

A further analysis was performed using Circular Dichroism. Circular Dichroism (CD) is sensitive to the nature of protein secondary structure. Since this technique is based on circularly polarized light, it is capable of distinguishing D- and L-protein enantiomers. As expected, mirror image CD spectra from correctly folded VEGF enantiomers was observed in experimentally obtained spectra. Both spectra are consistent with a largely beta-sheet conformation, and opposite amplitudes were observed, indicating that mirror-image secondary structure is present.

VEGF was also analyzed by analytical HPLC. Purified folded D-VEGF was analyzed using a Vydac C4 column and a water/acetonitrile mobile phase. At an absorbance of 214 nm, a peak was observed corresponding to a retention time of approximately 9.5 minutes.

Size Exclusion Chromatography was employed to further analyze D-VEGF. The protein was analyzed on an Akta FPLC system using a Superdex 75 column The majority of the protein elutes in a single peak near 11 mins (absorbance of 280 nm), consistent with the expected molecular weight of approximately 24 kDa. A smaller peak was also detected at the void volume, near 7 mins, which may be due to a small amount of aggregated protein.

Finally, chemical shift analysis of D- and L-VEGF's was performed and the chemical shift positions of L and D-VEGF amide fingerprint cross peaks were plotted against each other. Ideally, for mirror image proteins, the peaks should fall along a line with slope 1 passing through the origin. In the current results produced a line equation of y=−0.0079801+1.0011x (experimental, R=1, y intercept=−0.008 ppm), which is close to the ideal case.

### Example 3

### Combinatorial Peptide Selection

D-VEGF was used with enhanced combinatorial libraries to discover L-peptides that bind. By symmetry, the corresponding D-peptides will bind to native L-VEGF targets with identical affinities. Two, state-of-the-art, self-replicating libraries, gene-shuffled phage display and nucleic acid-peptide fusion libraries, have been evaluated. It should be noted that other library methods may also be used in accordance with the methods of the invention. At the outset of the project it was unclear which of these two methods would allow the most thorough exploration of sequence space and maximize the odds of finding rare high affinity VEGF binding sequences. In order to be competitive with antibody-based therapeutics, peptide ligands were required to bind selectively with maximum dissociation constants in the nanomolar range. Peptide selection experiments commenced immediately following the completed synthesis and characterization of the protein VEGF enantiomers. Peptides with dissociation constants in the low nanomolar range were discovered and advanced forward to the next task, in-vitro evaluation. In practice, it was found that nucleic-acid peptide fusion libraries yielded greater numbers of tighter binding peptide ligands for evaluation by in vitro assays. Therefore nucleic acid-peptide fusion libraries were focused on for the remainder of this project.

### Experimental Design

Site-specifically biotinylated D-VEGF was immobilized onto streptavidin beads at two different concentrations (100 nM as a starting point and 1 nM for late rounds of selections) and exposed to an mRNA-peptide fusion library, pp27 with a 27 residue variable region. Various sizes of variable regions may be used in accordance with the methods of the present invention, e.g., 5, 10, 12, 15, 20, 25, 30, 35, 40, or more residues may be in a variable region. Indeed, this may include any single length of variable region within the range of 4-40, e.g., 9 or 13, etc.

Fusions with affinity for the immobilized protein become associated with the beads, while non-binders are washed away. The nucleic acid portion of the fusions that survive the selection is then amplified using PCR. At this point this new pool of DNA can either be analyzed to determine the sequences of the fusions present, or it can be used to generate a new library of mRNA-peptide fusions for further rounds of selection.

### Results

pp27 vs. 100 nM D-VEGF:

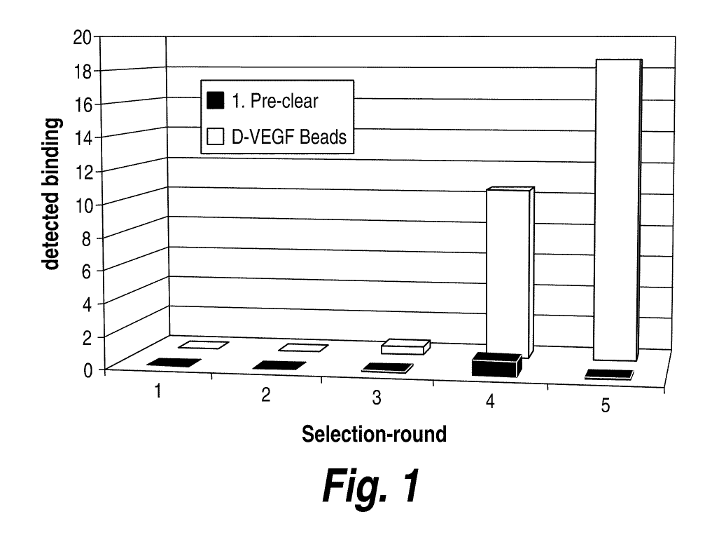

Enrichment of the mRNA-fusion pool became apparent after selection round 3. The percentage of pool binding increased in rounds 4 and 5 to over 18%. RNA-fusion binding to D-VEGF is shown graphically in FIG. 1. Prior to selection against D-VEGF, pools are depleted of biotin binding sequences by a “pre-clear” experiment in which the pool is exposed to streptavidin beads loaded with biotin only. The amount of RNA-fusions binding in the pre-clear is shown as a control in blue. 95 clones were sequenced from the DNA after pools 3, 4 and 5. A shift in sequence populations was observed, with two sequences dominating in the later pools. These two sequences contained a similar motif. Each peptide sequence was assigned a unique number for identification of biotinylated and non-biotinylated forms. The numbers are included below for future reference (lower numbers in the pairs correspond to non-biotinylated peptides). Biotinylated peptides were used for ELISA assays, and the non-biotinylated ones for in vitro assays which were subsequently initiated. Among the 95 peptides were found several peptides that shared a common motif, of which particular note was taken. Sequences based on G2211/G2226 were found to bind to VEGF165 selectively by a variety of methods (see below). Sequences derived from the other clones were not found to bind or bound in a non-specific manner and were not pursued further in this set of experiments.

| | |
|---|---|
| GVQEDVSSTLGSWVLLPFHRGTRLSVWVT | (SEQ ID NO: 28) |
| G2211/2226 | |
| GAGLWWGFCTDQHCIFKSPTLSSFVIVDT | (SEQ ID NO: 29) |
| G2212/2227 | |

pp27 vs. 1 nM D-VEGF

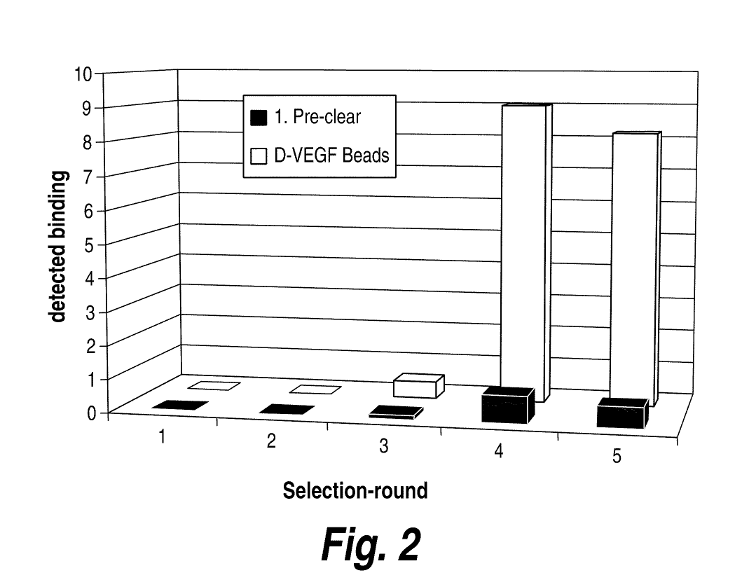

In an effort to find peptide ligands with higher affinity for D-VEGF, the pp 27 RNA-peptide library was used in a selection against 1 nM immobilized D-VEGF. As shown in FIG. 2 (see the lighter bars toward the back of the 3-D plot), pool binding was detected after round 3 of enrichment and increased to 9% of the total pool after 4 rounds of selection. At round 5, no increase in binding was found, so the selection was stopped after round 5. Bars in the foreground of the 3D plot indicate the binding to pre-clears against biotin loaded streptavidin beads.

95 DNA clones from each pool following rounds 3 through 5 were sequenced. In addition to the dominant sequences found in the selection against 100 nM D-VEGF, two additional sequences were found in relatively high abundance in rounds 4 and 5 of the new selection against 1 nM protein. Designator numbers and sequences are shown below for two peptides from this pool that were given particular scrutiny. There were indications from equilibrium bead binding, BIACORE, receptor ELISA and cell based assay experiments that sequences based on G2257/2258 were of particular interest.

| | |
|---|---|
| GGFEGLSQARKDQLWLFLMQHIRSYRTIT | (SEQ ID NO: 26) |
| G2255/2256 | |
| GNALHWVCASNICWRPPWAGRLWGLVRLT | (SEQ ID NO: 30) |
| G2257/2258 (07-D60) | |

It is understood that peptide selection experiments may not yield bioactive peptides and extensive searching of the libraries may not yield peptides with the requisite affinity or selectivity. As expected, the majority of peptides tested do not appear to be bioactive, however sequence G2211/2226 appears to bind tightly to VEGF165 and 07-D60 appears to inhibit HUVEC cell growth in a cell based assay. Therefore, despite the difficulty of identifying bioactive peptides, the experimental procedure was successful in discovering several peptides which bind VEGF with low nanomolar affinities and do so in a selective manner.

One concern at the start of these experiments was that isolated peptides may not exhibit the same behavior as the same sequences encoded onto viral coat proteins or as peptide-nucleic acid fusions, as present in the libraries. This appeared to be a more pronounced issue in the enhanced phage display experiments compared to the nucleic acid-peptide libraries. This may be due in part to the relatively large size of the phage particles, or perhaps due to small numbers of multivalent phage particles affecting the outcome of selection experiments. Several strategies were employed to mitigate these effects. The possibility of RNA-based binding in the nucleic acid-peptide fusion libraries was reduced by carrying out selection experiments in the presence of salmon sperm DNA. Highly ionic nucleic acids may also aid in solubilizing hydrophobic peptide sequences. Polymer modifications will continue to be used to minimize activity differences derived from lack of solubility.

### Example 4

### Synthesis of D-Peptides

The D-enantiomers of the peptide ligands that were enriched in the selection experiments were chemically synthesized. Since the peptides of interest were typically relatively small in this set of experiments (variable regions˜15-27 residues in length) it was straightforward to obtain these materials in milligram quantities for initial biological screening. Peptide synthesis cycles essentially identical to the ones described above for the synthesis of peptides were used for assembly of synthetic proteins. It was found to be useful to add short polymeric groups to most peptide sequences to improve their solubility in common buffers. Peptides were purified by RP-HPLC and characterized by ES-MS and analytical HPLC before use in in vitro assays in Example 5.

Further peptides may be synthesized in a similar manner using various subcontractors, or the peptide facility at the Cosmix Molecular Biologicals site in Braunschweig. Promising sequences may later be subjected to a routine “format analysis” in which various polymer and ionic groups are used to determine which provide optimal solubility and activity in in vitro assays.

It was observed previously that polymer modification can significantly influence peptide binding properties. To minimize this effect in the future, small groups of structurally related peptides in which the polymer and ionic modifications are systematically varied will be prepared for testing.

### Example 5

### In Vitro Evaluation

The binding properties of the selected D-peptides were characterized on native VEGF 165 with equilibrium binding, ELISA, and BIACORE experiments. D-peptides that exhibit low nanomolar binding were evaluated most extensively. A factor-dependent cell proliferation assay was used to evaluate anti-VEGF activity, and HUVEC-based assays will continue to be used to test the efficacy of peptides. This experiment has identified one sequence that appears to be active in inhibiting HUVEC cell growth in this assay.

Peptide synthesis cycles were employed that were essentially identical to the ones described above for the synthesis of peptides for assembly of synthetic proteins. Dissociation constants will be confirmed by ELISA and biosensor methods carried out at Cosmix. The bioactivity of D-peptides may be probed later using cell-based assays. Peptide leads that exhibit in vitro bioactivity will then be tested with in vivo animal models to select pre-clinical development candidates.

D-peptide binding characteristics were initially investigated and their potential for disruption of cytokine-receptor binding was assessed. Binding isotherms resulting from BIACORE experiments with immobilized D-peptides and L-proteins in the flow solution were used to confirm dissociation constants obtained from ELISA experiments on the L-peptides. NMR fingerprinting techniques may further be used to probe the location and binding specificity of the D-peptide sequences. To accomplish this, HSQC fingerprints of the amide NH region may be collected in the absence of D-peptide. These fingerprints may then be compared to spectra collected in the presence of increasing quantities of D-peptide.

A cell-based assay for anti-VEGF activity was carried out using a factor-dependent cell line whose proliferation is sensitive to VEGF. Holash and coworkers describe the use of transfected NIH 3T3 cells with a chimeric receptor featuring the extracellular portion of VEGFR-2 fused to the cytosolic domain of TrkB, which drives cellular proliferation upon activation. Serial dilutions of candidate anti-VEGF D-peptides will be assayed in parallel with a neutralizing anti-VEGF antibody (R & D Systems) as a positive control a blank buffer sample and a sample of a D-peptide with a scrambled sequence as negative controls. After incubation for 72 hrs, growth was either measured colorimetrically or by scintillation counting. Various methods may be used. For example, in one radiometric method inhibition constants (ki's) were determined from a four parameter fit of cell counts as a function of D-peptide concentration.

### ELISA Assay

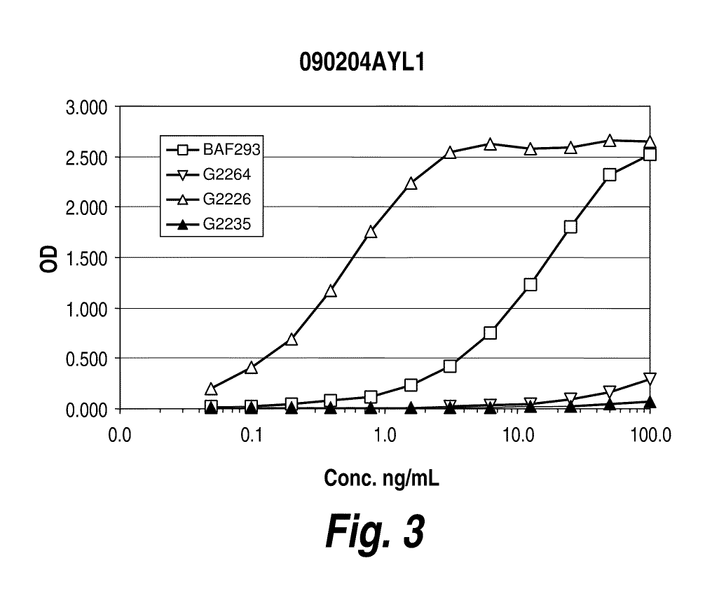

An ELISA method was developed in which commercially available VEGF165 was coated into ELISA plate wells (MaxiSorb). Biotin-tagged D-peptides (or an anti-VEGF monoclonal antibody, BAF293, R & D Systems) were then added. After washing binding of peptides or antibody to VEGF was determined by treatment with a streptavidin/HRP conjugate which allows colorimetric detection of the presence of the biotin tagged peptides or antibody (see FIG. 3).

This experiment indicates that a peptide selected against the D-VEGF receptor-binding domain (residues 8-109), G2226 appears to bind tightly to VEGF165, the most commonly occurring natural isoform. This assay may be used in the future to investigate the binding properties of peptides sequences resulting from lead optimization efforts (such as in Example 6).

### Equilibrium Bead-Binding Assay

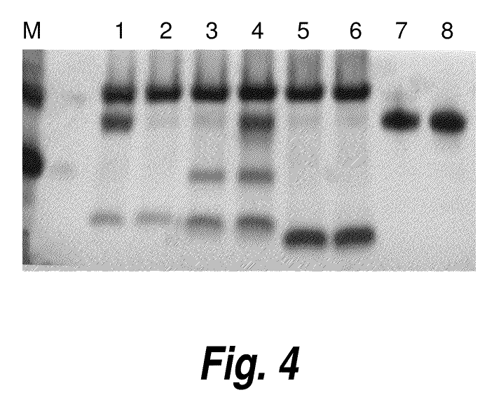

A quick, qualitative binding assay was devised in which biotin-tagged peptides are immobilized onto streptavidin-coated magnetic beads. The bead-bound peptides are then treated with VEGF (either synthetic or commercially available VEGF 165). After washing the beads are then boiled in denaturing buffer to release bound VEGF which is then detected by silver-stained SDS-PAGE gel (see FIG. 4).

This test was used to evaluate the stereoselectivity of G2226 binding. G2226 was found to bind to synthetic L-VEGF as well as commercially available VEGF 165, however it was found to have no detectable affinity for D-VEGF. These experiments demonstrate that G2226 binds to L-VEGF in a stereoselective manner. Peptide G2248 was also found by the bead binding assay to have considerable affinity for L-VEGF, however it also appeared to bind D-VEGF, indicating a lack of stereospecificity. This assay may be used to survey the binding properties of peptides sequences resulting from our lead optimization efforts (Example 6).

### BIACORE Measurements

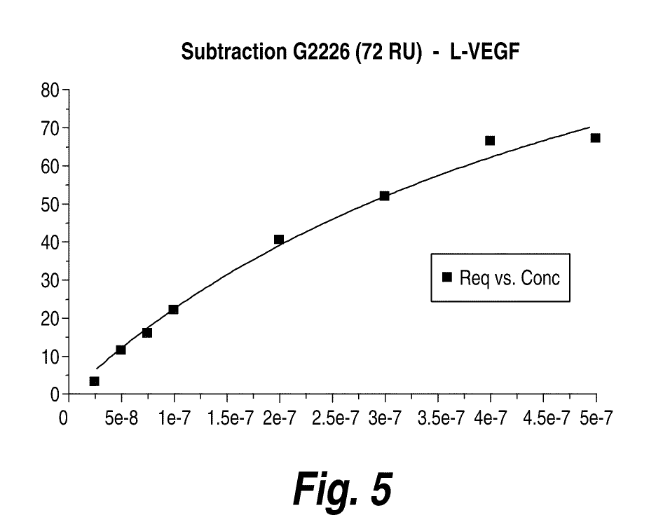

Biosensor experiments were carried out using a BIACORE instrument. D-peptides were immobilized onto a streptavidin coated chip. VEGF containing solutions were flowed over the surface of the chip using the BIACORE's microfluidics system, and binding events were detected by measuring the surface plasmon resonance effect. Dissociation constants can be calculated from the apparent on and off rates obtained from the BIACORE data. Different data sets gave varied results for G2226 dissociation constants, however, it is likely below 20 nM (see FIG. 5)

Because of the lower inherent throughput of BIACORE measurements compared to the bead-binding and ELISA format experiments, we will limit the use of BIACORE to further characterize peptide sequences that appear promising by other ELISA and bead binding methods.

### Receptor ELISA

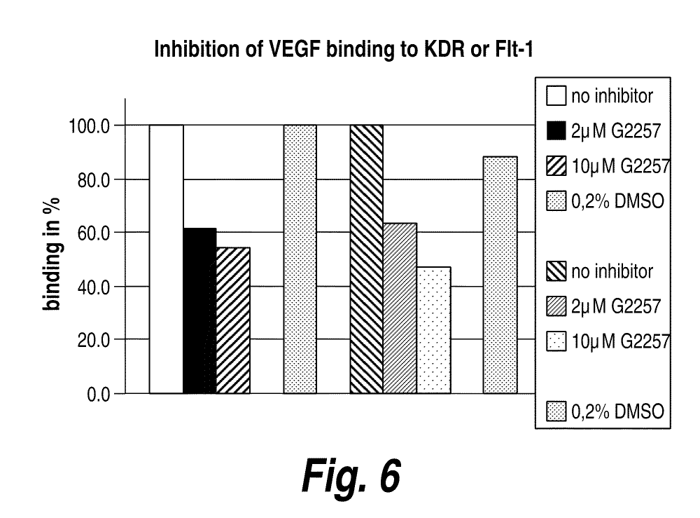

VEGF acts on two distinct receptors in-vivo, Flt-1 (VEGFR1) and KDR (VEGFR2). The majority of pro-angiogenic signaling appears to be mediated by KDR. Flt-1 binds to VEGF isoforms with a higher affinity than KDR and may serve a largely regulatory role in vivo. Certain D-peptide sequences (e.g., G2257) have now been shown to be capable of inhibiting VEGF binding to KDR. These studies have been extended using ELISA assays on VEGF Flt-1 binding. In this experiment a soluble fusion of Flt-1 was coated onto Costar ELISA plates. After blocking (2% BSA), solutions of rh-VEGF165 containing serial dilutions of peptide test articles were added. Detection was accomplished using a biotinylated anti-VEGF antibody in combination with a streptavidin-HRP conjugate. The results of the Flt-1 assay are shown in FIG. 6 along with previous results from the analogous KDR assay for comparison.

The results of the ELISA show that G2257 inhibits binding of VEGF165 to both receptors. This is consistent with an effect resulting from peptide binding to VEGF as opposed to possible non-specific binding of the peptide to the surface of the receptors. While the regions of VEGF that are responsible for binding Flt-1 and KDR are distinct, they have considerable overlap. From the ELISA data it is likely that G2257 binds an epitope on VEGF that includes both Flt-1 and KDR binding regions.

Due to the relatively high cost of this ELISA format, we will limit its use to characterize peptide sequences that appear promising by antibody ELISA, bead-binding and BIACORE experiments.

### HUVEC Cell-Based Assay

While ELISA-type assays are valuable for determining which peptides are capable of inhibiting VEGF binding to its cognate receptors and useful for obtaining thermodynamic parameters related to binding, they do not directly evaluate the in vivo (i.e., cellular based) biological activity of their test articles. Initial investigation into the biological activity of the peptides involved use of a HUVEC cell-proliferation assay. A contractor (ReliaTech, Braunschweig, Germany) with considerable experience in this assay was employed to carry it out. ReliaTech's assay employed a radiation-based system to detect cell growth by monitoring DNA synthesis using tritium-labeled thymidine.

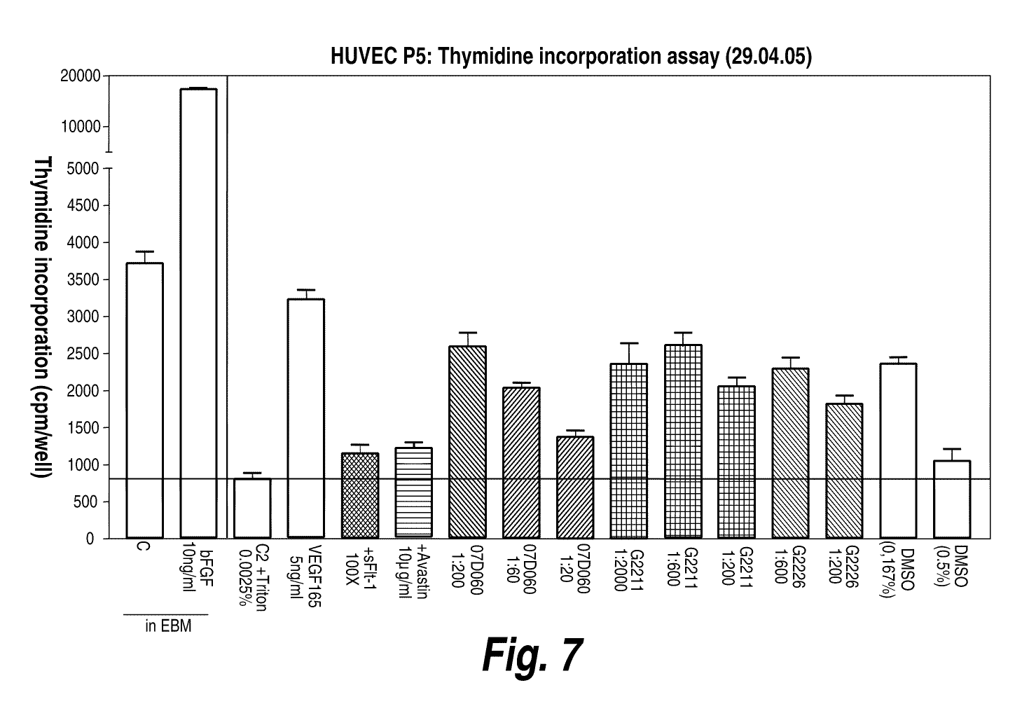

In this assay, pregrown, starved HUVEC cells proliferate in response to exposure to VEGF or bFGF. Inhibition of VEGF activity will result in a measurable decrease in cell proliferation. Peptide 07-D60 (a modification of G2257) appears to block HUVEC growth in a dose-dependent manner. Avastin (Genentech's anti-VEGF antibody) was used as a positive control and showed strong inhibition of HUVEC growth. The effects seen for other peptide sequences were attributed to the DMSO content of their stock solutions (DMSO was required to solubilize all peptides in this assay except for 07-D60). At 10 micromolar concentration, 07-D60's inhibition approaches that for 67 nM Avastin. The results are shown in FIG. 7.

Several difficulties were overcome while performing the experiments described above. First, it was originally thought that colorimetric cell-based assays may not be precise enough to discriminate between similar D-peptide inhibitors. As expected, the colorimetric assays were not as effective as the scintillation-based assays. The additional cost of the scintillation assays was found to be worth the added reliability and precision.

It was also considered that some of the peptide sequences may not be readily soluble in the absence of viral coat protein or nucleic acids. Indeed, it was found that many of the peptide sequences found from nucleic acid-peptide library selections were quite hydrophobic and quite insoluble in many common buffers. To counteract this, a short polymer tag (defined length polyethylene glycol) was added to the core peptide sequences. In addition, experiments were undertaken to find buffer conditions compatible with our assays that improved solubility of the peptides.

A further concern was that measured dissociation constants for peptide binding may not necessarily correlate with inhibition constants. For example, sequence G2226 had the best affinity for VEGF165 and bound in a selective manner, however it does not appear to be active in cell-based assays and moreover does not appear to block VEGF binding in the receptor ELISA. We cannot, a priori, predict whether observed inhibition constants will correlate with the dissociation constants, however, we will attempt to optimize the activity of lead peptides that do show inhibitory effects by affinity maturation (see below). Peptides which show little or no inhibitory activity may possibly be used in diagnostic assays or for delivery of secondary molecules/therapeutics

### Example 6

### Lead Optimization

Although some identified peptides may not initially exhibit the desired binding properties or inhibitory activity, it is possible to alter or mutagenize the peptides to produce more favorable attributes. One method is to use a soft mutagenesis approach to peptides found to inhibit HUVEC cell growth (for example, peptide 07-D60) and evaluate the properties of the mutant progeny. Mutants with improved properties are selected and subject to further rounds of soft mutagenesis until target properties fall within a predetermined range (e.g., having a low nanomolar dissociation constant and <100 nM inhibition constant). In doing so, a constrained search is being carried out of peptide sequence space near a sequence with known activity. By examining related sequences the bioactivity will be refined by locating nearby sequences that represent global thermodynamic minima for target binding. It is anticipated that such an optimization may take approximately 3 months on a candidate peptide such as 07-D60.

A candidate, e.g. a peptide such as 07-D60 that is able to block HUVEC growth in a dose-dependant manner, should be chosen for optimization. The peptide should to be mutagenized in a such a way that the affinity for VEGF is improved but not at the expense of losing its ability to block HUVEC growth by binding to another site on the VEGF molecule. Therefore, a moderate mutagenesis approach is required. By creating a new library derived from a peptide, e.g., 07-D60, in a way where each amino acid position is mutated once, on average, the chances to improve the affinity is high without losing the original binding site required for the blockade of VEGF signalling. Furthermore, methods such as those in U.S. Pat. Nos. 5,798,208; 5,830,650; 6,649,340; and U.S. patent application Ser. No. 10/877,467 (which are incorporated herein by reference) could potentially be employed.

To achieve this initial goal a moderate PCR approach as well as a mutagenesis kit from Invitrogen was used, corresponding clones are sequenced and analyzed in order to find the most suitable way to generate a peptide 07-D60 derived library having one or two point mutations compared to the original clone. Such a library will serve then as a starting point to perform several selection cycles under more stringent conditions like increased washing times (overnight), small target concentrations (1-2 nM), and an excess of non-immobilized target to reduce rebinding effects to a minimum. Enriched pools after five more rounds of selections are cloned, sequenced, and analyzed and should yield sequences with only a moderate number of mutations when compared to the original clone. The identified sequences are then synthesized as free peptides and analyzed by VEGF-binding assays, competition assay, BIAcore analysis, and finally with a HUVEC cell growth assay. It is expected that these new sequences have improved affinities compared to the original peptide, and that a 10-20 fold improvement in affinity is possible.

### Introduction to Examples 7-10

One promising candidate peptide, G2306, was chosen as a lead in the following examples since it had proven to be an excellent candidate in terms of affinity and biological activity as it has been shown in various assays like ELISA, receptor competition, bead binding, and other assays. In addition, peptide G2306 showed an effective dose-dependent inhibition in a VEGF-dependent cell growth assay. As described in the following examples, G2306, was subjected to a controlled mutagenesis. The aim of this mutagenesis was to apply defined moderate conditions to allow for one or two mutations to occur within the overall sequence.

The rationale to keep the mutation rate very low in some examples was that it is desired to improve the affinity towards VEGF but not to loose the actual binding site of the peptide on VEGF since the peptide binding site appeared, in this case, to be either identical or very close to the natural binding site of VEGF to its receptor. One of skill in the art will understand that various mutagenesis strategies may be employed, and that higher or lower rates of mutagenesis may be desired in other embodiments.

For the mutagenesis described in this example, various parameters were examined in regard to the controlled mutagenesis by PCR. Such parameters are the polymerases to be used, temperature, template concentration, primer concentrations, presence of divalent cations, and the base composition of the template. A working pool of mutated sequences derived from the original clone G2306, and in general exerting not more than one or two mutations within the original sequence, has been sized up as starting material to undergo additional selection rounds under more stringent conditions by mRNA display to improve the affinity towards VEGF. Using this starting material an independent selection was performed with the following measures to increase the affinity of resulting peptide binders towards D-VEGF as target. This is referred to as Selection 1 and is described in greater detail in Example 8.

The amount of target during the selection was lowered to 5 pmol. The stringency of the overall selection was significantly increased by washing the samples 10 times with one wash lasting overnight on a tumbler. Non-immobilized D-VEGF (at least 10-fold excess over immobilized target) was used to compete out low affinity binders.

The application of these conditions resulted in a significant drop of the binding signal back to almost background level during the first round of the selection, as discussed below. This was expected since the original sequence had been mutated and the stringency during the selection was significantly increased by overnight washings. By monitoring the binding signal during consecutive rounds of the selection the binding signal not only came back, moreover, it showed a significant increase of about 4% net binding after round 4 of the selection.

As a back up for the selection 1 another selection was performed, as described further in Example 9, where the same measures as in selection 1 were performed except for one major difference: In each selection round the library pool was subjected to a mutational PCR (in the presence of 2.5 nM Mn2+) with non-stringent and mutating conditions. By monitoring the binding signal during consecutive rounds of the selection the binding signals obtained during this selection were highly comparable to those found in selection 1 with a significant increase of about 8% net binding after round 4 of the selection.

For both selections, pools of resulting peptide binders were recovered, cloned, sequenced and analyzed. The sequence analysis revealed that almost most of the resulting matured sequences share high homology between selections 1 and 2, and they contain three different hot spot mutations when compared to the starting sequence of G2306.

For initial analyses, two variants of the resulting sequences from selection 1 were subjected to chemical synthesis as L-peptides and analyzed in an ELISA format with the original sequences of G2306 as a control. As judged from the ELISA analysis at least one of the two variants tested, 07-072, a variant carrying three hot spot mutations, shows a vast improvement towards the affinity of D-VEGF when compared to the corresponding starting sequence of G2306.

ELISA assays have been extended and been found to confirm the initial results. The synthesis of various L-peptides to be used as controls during the animal assays has also been completed and they reveal that all different formats chosen are readily soluble which is a good prerequisite for their use in animal says.

It should be understood by the skilled artisan that the mutagenesis, selection, and affinity maturation strategies described in examples 7-10 are only particular embodiments. mRNA display technologies are well known in the art and various mRNA display methods may be used in accordance with the present invention, e.g., the display methods referenced in the present description.

### Example 7

### Affinity Maturation and Library Preparation

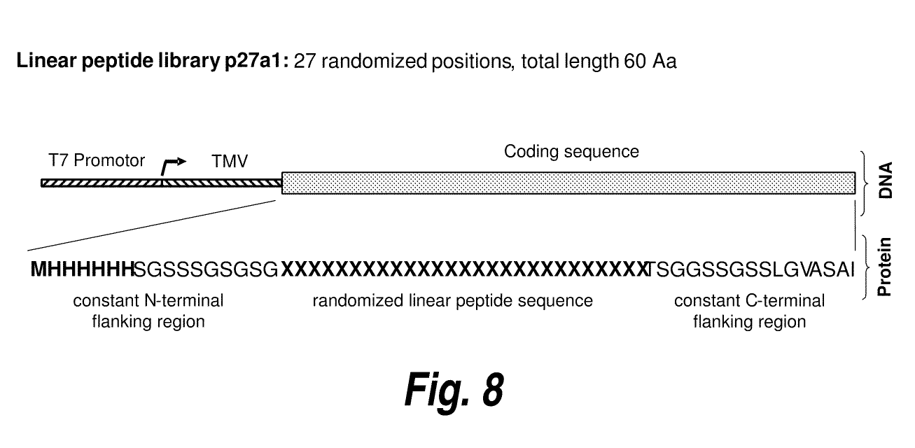

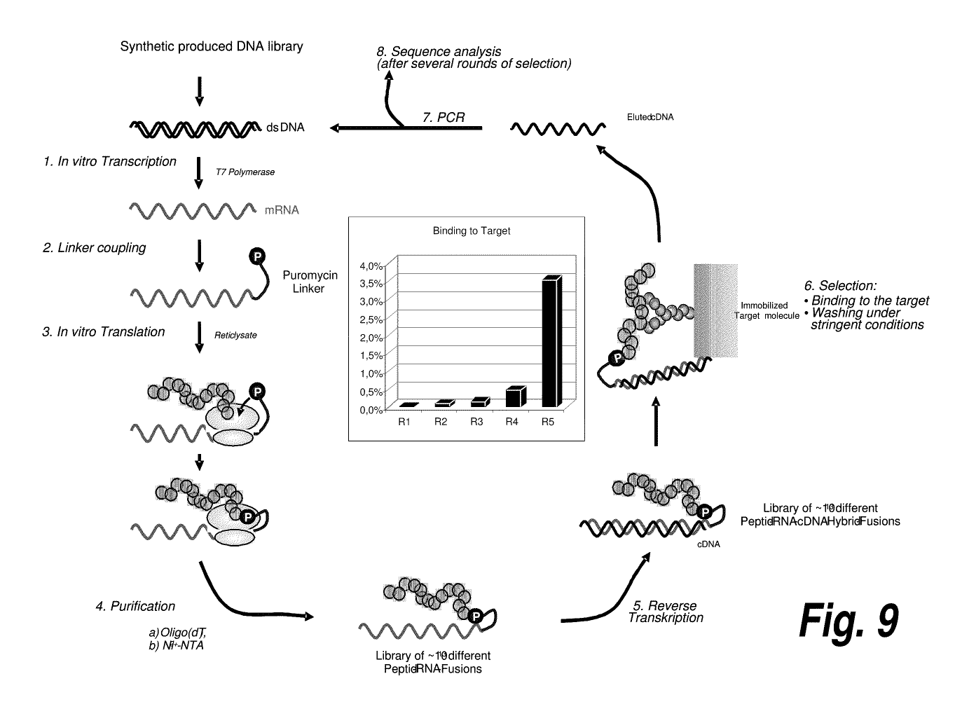

As starting material for this mRNA display selection program mutated binder pools were derived from the original peptide G2306 from a linear peptide library containing 27 randomized amino acid positions. Structure of the originally randomized library is given in FIG. 8. An overview of mRNA display selection technology is shown in FIG. 9 and described below in more detail:

As starting material for this mRNA display selection highly purified p27a1 RNA from the mutated pool of G2306 derived-L-candidates was used.

The RNA was modified by attachment of a Puromycin-like linker molecule to the 3′-end (covalent coupling achieved by irradiation with UV-light) and translated in vitro by means of a rabbit reticulocyte lysate in the presence of radioactive ³⁵S-Methionine.

During this step covalent fusions between translated peptides and coding RNAs were simultaneously formed. Fusion molecules were purified from the translation reaction mixtures on magnetic oligo(dT) beads, reverse transcribed and finally purified by Ni²⁺-metal-affinity chromatography and could then be used for selection steps. About 1.6 pmol of peptide-RNA-cDNA-fusions were used as input for the first contact with target material (biotinylated D-VEGF immobilized on Streptavidin beads) during selection round 1.

### Example 8

### Selection—Binding to Target Protein and Washing

The selection process was performed at 4° C. after dilution of purified peptide-RNA-cDNA-fusions in HNT buffer containing 1 mg/ml BSA and 0.1 mg/ml sheared salmon sperm DNA and structured into three steps:

Preclearing: Preclear/removal of undesired binders by repetitive incubations during all selection with unloaded M280-Streptavidin beads or Biotin-saturated M280-Strepatvidin beads.
Binding reaction: Enrichment of desired binders by incubation with biotinylated D-VEGF immobilized on magnetic M280-Streptavidin beads in the presence of a 10-fold excess of non-immobilized D-VEGF to compete out low affinity binders.
Washing procedure: Removal of unspecific or weak binders by applying various washing steps (10×) including washes to target-loaded beads overnight in a tumbler.

After washing selection beads were resuspended in 50 μl of water and could directly be transferred into PCR to allow amplification of cDNA of enriched binder variants. After PCR amplification, the selected cDNA was analyzed and cloned (e.g., by transformation into E. coli host cells). Colonies positive for transformation of the chosen vector were then be selected for sequencing. The peptides were then synthesized according to the Fmoc/But strategy by SHEPPARD and further purified.

### Example 9

### Results and Analysis of Affinity Maturation Selection of Peptide Binders Against D-VEGF

During all rounds of mRNA display selections biotinylated D-VEGF immobilized on magnetic Streptavidin beads was used as target protein. In order to minimize the simultaneous enrichment of bead- and/or Streptavidin-specific binders the pool of binder candidates was intensively cleared by repetitive incubations with target-free Streptavidin beads before contacting with D-VEGF-loaded beads during every round of selection.

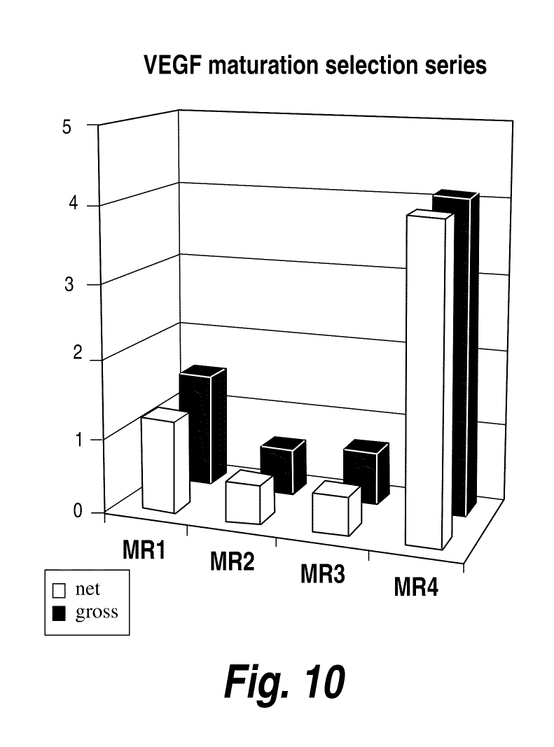

The incorporated radioactive labeled Methionine in peptide-RNA-cDNA-fusions did allow a direct monitoring of fusions binding to target-free as well as to target-loaded beads by scintillation measurement. Up to the third round of selection we observed a decrease in the binding signal. During selection rounds MR2, MR3 and MR4 we implemented a competition with a five-fold excess of non-immobilized D-VEGF over target concentration to reduce low affinity binders (see FIG. 10). Moreover, the washing conditions were significantly raised in comparison to our standard selection by repeating the washing 10 times during each selection round and include one washing overnight by rotating the sample on a tumbler.

Therefore this selection pressure should especially favor the enrichment of high affinity binders to D-VEGF while binders of moderate affinity are expected to be lost. At the same time the applied selection pressure did dramatically favor the enrichment of high affinity D-VEGF specific binding variants.

cDNAs of enriched binder pools from the 4^{th} round of selection were cloned after PCR amplification and gel purification and subsequently identified by sequence analysis (see FIG. 11).

As shown in Table 2 nearly all enriched binder sequences share some hot spot mutations compared to the starting sequence of G2306.

TABLE 2

| | | |
|---|---|---|
| wt | NALHWVCASNICWRSPWAGRLWGLVRL | (SEQ ID NO: 20) |
| 37x | NALHWVCASNICWRTPWAGRLWGLVRL | (SEQ ID NO: 21) |
| 29x | NALHWVCASNICWRTPWAGQLWGLVRL | (SEQ ID NO: 22) |
| 14x | NALHWVCASNICWRTPWAGRLWRLVRL | (SEQ ID NO: 23) |
| 8x | NALHWVCASNICWRTPWAGRLWELVRL | (SEQ ID NO: 24) |

Table 2: Frequency of mutation occurrence within peptide-RNA-cDNA fusions after selection on D-VEGF After PCR-amplification, ligation into plasmid pSTBlue-1 and cloning in E. coli the encoding cDNAs of enriched binder pools after selection round 4 was subjected to sequence analysis. The frequency of occurrence of the corresponding Amino acid changes within the sequences are listed in the table. Analysis has been based on a total number of 59 clones.

### Example 10

### Back Up—Affinity Maturation Selection of Peptide Binders Against D-VEGF

As a back up for the selection 1 another selection was performed where wherein the following measures from selection 1 were maintained:

The amount of target during the selection was lowered to 5 pmol.

The stringency of the overall selection was significantly increased by washing the samples 10 times with one wash lasting overnight on a tumbler. Non-immobilized D-VEGF (at least 10-fold excess over immobilized target) was used to compete out low affinity binders.

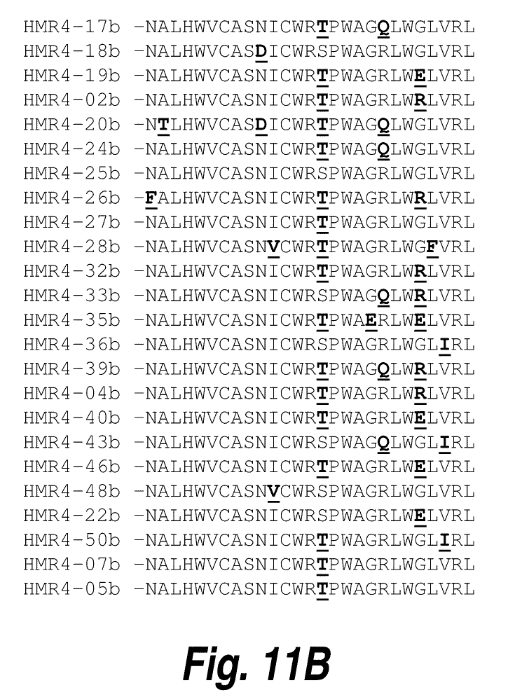

The major difference when compared to selection 1 was that in each selection round the library pool was subjected to a mutational PCR with non-stringent and mutating conditions. The presence of 2.5 nM Mn²⁺-ions during the PCR reaction is known to drive the mutation rate of the resulting DNA fragments. The application of these conditions resulted in a significant drop of the binding signal back to almost background level during the first round of the selection. This was expected due to the high stringency during the selection like overnight washings and drop of target concentration. By monitoring the binding signal during consecutive rounds of the selection the binding signals obtained during this selection were highly comparable to those found in selection 1 with a significant increase of about 8% net binding after round 4 of the selection. Consequently, this pool of resulting peptide binders was recovered, cloned, sequenced and analyzed. The sequence analysis revealed that almost all resulting matured sequences share high homology to the sequences found during selection 1 with three different hot spot mutations when compared to the starting sequence of G2306. See FIG. 12, which depicts the enrichment of D-VEGF binders over several rounds of display, and see FIG. 13 for many of the enriched binder sequences found. FIG. 14 depicts the most common variations seen in the selected binders.

As it can be seen from the sequence data in FIG. 13 and FIG. 14, mutations appear more frequently throughout the sequenced colonies although the same hot spot mutations as found during selection 1 dominate clearly the pool of various sequences identified.

One of skill in the art will appreciate that any mutational procedure known in the art would be compatible with the described selection methods. Examples of mutational strategies may be found in U.S. Pat. Nos. 5,932,419; 5,789,166; 6,132,970; 5,556,747; 6,153,410; 6,180,406; 5,466,591; 5,108,892; 6,303,344; 5,223,408; 5,830,721; 5,512,463; 6,171,820; 4,959,312; 5,798,208; 5,885,827; 5,830,650; and U.S. patent application Ser. Nos. 10/943,511; 10/877,467; 10/491,620; 11/762,580; 11/736,803; 11/562,849; and 10/573,639 which are all incorporated herein by reference.

### Example 11

### Synthesis of L-Peptide Variants and ELISA Assays

Based on Table 2 two variants were subjected to chemical synthesis as L-peptides for an initial analysis to find out whether the affinity maturation has been successful or not.

TABLE 3

| Pep- | |
|---|---|
| tide | Sequence |
| 07-007 | Ac-GNALHWVCASNICWRSPWAGRLWGLVRLT-PEG-Bio |
| | (SEQ ID NO: 11) |
| 07-071 | H₂N-SGSSSGSGSGNTLHWVCASDICWRTPWAGQLWGLVRLT- |
| | PEG-Bio |
| | (SEQ ID NO: 12) |
| 07-072 | H₂N-SGSSSGSGSGNALHWVCASNICWRTPWAGQLWRLVRLT- |
| | PEG-Bio |
| | (SEQ ID NO: 16) |

Table 3: Peptide 07-007 resembles the original starting sequence of G2306 whereas peptides 07-071 and 07-072 carry mutational hot spots as found in the sequence pool of the final round of the selection. All peptides synthesized were in the L-form and had been synthesized on a small scale basis. All peptides were readily soluble.

ELISA plates were coated for 60 minutes at 37° C. with hu IgG 280 ng/well in PBS, L-VEGF 20 pmol/well in PBS, D-VEGF 20 pmol/well in PBS, 2% milk in PBS respectively and plates were consecutively blocked with 2% Milk in HBS for 30 minutes at RT. Then the plates were incubated with 25 nM of peptides 07-007, 07-071 and 07-072 respectively in HBS buffer for one hour at room temperature followed by 4× washes with HBS buffer. FIG. 12 shows the results from the ELISA analysis after detection with Streptavidin-Peroxidase stained with o-Phenylendiamin and H₂O₂ for 3 minutes.

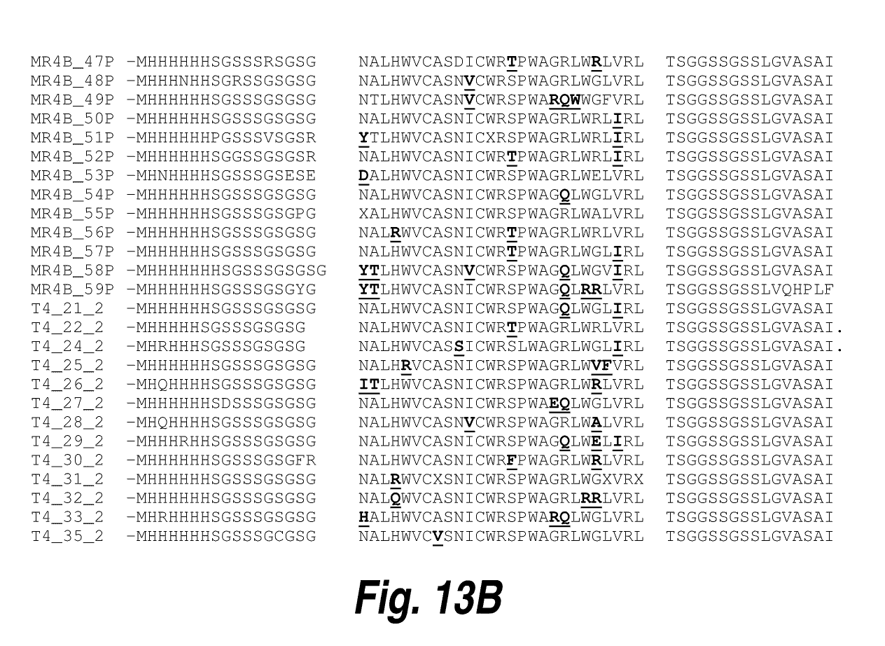

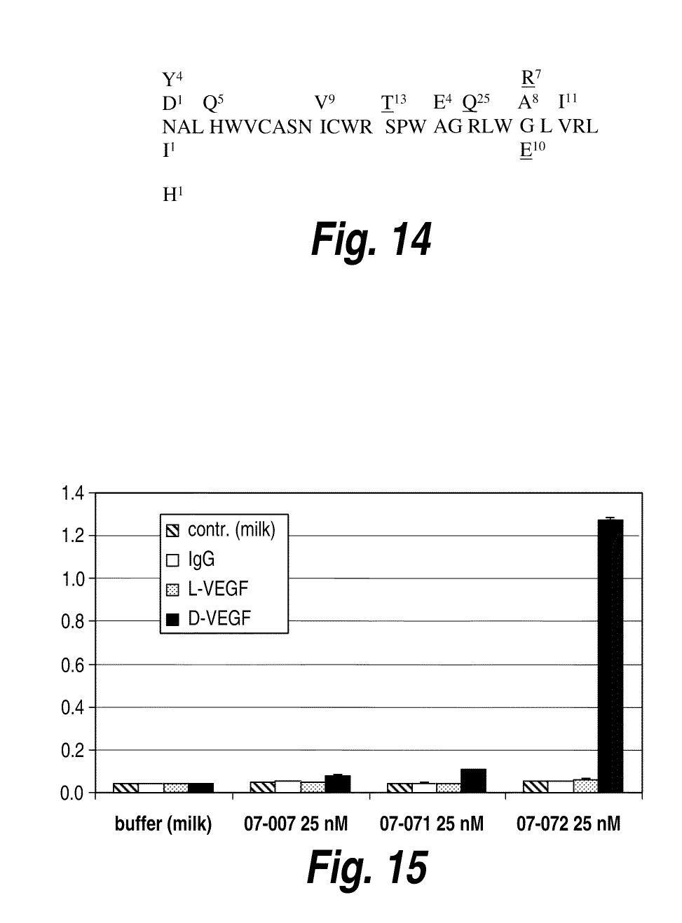

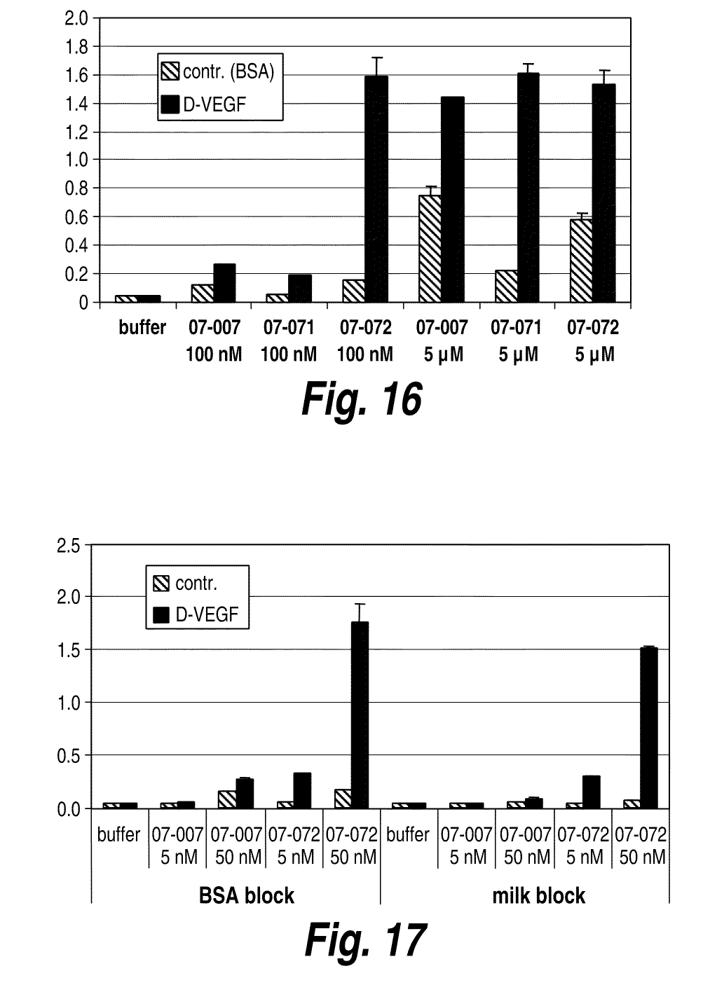

As shown in FIG. 15, peptide 07-072 carrying all three hot spot mutations within the matured sequence shows a vast improvement of the affinity towards D-VEGF in comparison to the non-matured sequence deriving from G2306. This result was verified by ELISA assays at different concentration ranges at 5M, 100 nM, 50 nM and 5 nM of identified L-peptides, washed and developed as shown in FIG. 16 (5M and 100 nM) and in FIG. 17 (50 nM and 5 nM). In all these ELISA assays the variant carrying all three hot spot mutations has a significantly increased signal when compared to the non-mutated sequence deriving from G2306. As judged from these ELISA analyses there is approximately at least a 10-fold improvement of affinity, reaching the picomolar range, with our peptides which is very promising in regard to therapeutic potential.

Based on the outcome of the selections as described above, peptide variants having the variant formats shown in Table 4 will be synthesized and analyzed further:

TABLE 4
Peptide Formats

| | | | PEG₂ | |
|---|---|---|---|---|
| Formats | n-terminus | aa-sequence | (Spacer) | c-terminus |
| 1 | H₂N— | L-X₃₇ | —NH-PEG₂- | —NH₂ |
| | | | CO— | |
| 2 | H₂N— | D-X₃₇ | —NH-PEG₂- | —NH₂ |
| | | | CO— | |
| 3 | H₂N— | D-X₃₇ | —NH-PEG₂- | —N-biotinyl-L-K—CO—NH₂ |
| | | | CO— | |
| 4 | H₂N-PEG₅₀₀₀- | D-X₃₇ | —NH-PEG₂- | —NH₂ |
| | CO— | | CO— | |
| 5 | H₂N-PEG₅₀₀₀- | D-X₃₇ | —NH-PEG₂- | —N-biotinyl-L-K—CO—NH₂ |
| | CO— | | CO— | |

The rationale behind the formats is the following. The L-versions which are used as control peptides should not exert any effects. The pegylation using two PEG units at the C-terminus will help with the solubility of the peptides. The Lysine (K)—versions will be used for an easy detection of the peptide in blood samples necessary to perform some of the planned studies. In addition to a normal format we will introduce a heavy PEG unit at the N-terminus of the peptides; this will prevent a rapid clearance of the peptides through the kidney since the cut off is known to be around 12 kDa. See Table X below for depiction of Fmoc-NH-PEGₙ-COOH.

TABLE 5
Structure of Fmoc-NH-PEGₙ-COOH

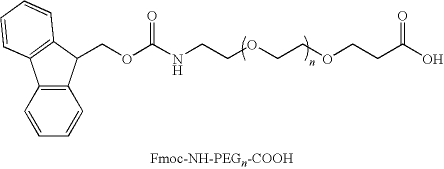

### EQUIVALENTS

Those skilled in the art will recognize, or be able to ascertain using no more that routine experimentation, many equivalents to the specific embodiments of the invention described herein. Such equivalents are intended to be encompassed by the following claims.

1. An isolated peptide or mimetic thereof which specifically binds to VEGF wherein the isolated peptide or mimetic thereof is between 4 and 90 amino acids in length and wherein the amino acids are D type optical isomers.

2. An isolated peptide or mimetic thereof, the peptide comprising the following formula (SEQ ID NO: 34):
F₁-Z₁-G-X₁-X₂-L-X₃-X₄-V-C-X₅-X₆-X₇-X₈-C-W-X₉-X₁₀-X₁₁-W-A-X₁₂-X₁₃-X₁₄-X₁₅-X₁₆-X₁₇-X₁₈-X₁₉-L-Z₂-F₂
wherein
X₁ is chosen from the group consisting of the amino acids N, Y, F, D, I, and H;
X₂ is chosen from the group consisting of the amino acids A, T, and V;
X₃ is chosen from the group consisting of the amino acids H, Q, and R;
X₄ is chosen from the group consisting of the amino acids W and R;
X₅ is chosen from the group consisting of the amino acids A and V;
X₆ is chosen from the group consisting of the amino acids S and L;
X₇ is chosen from the group consisting of the amino acids N, S, and D;
X₈ is chosen from the group consisting of the amino acids I, V, and H;
X₉ is chosen from the group consisting of the amino acids R and M;
X₁₀ is chosen from the group consisting of the amino acids S, T, P, and F;
X₁₁ is chosen from the group consisting of the amino acids P and L;
X₁₂ is chosen from the group consisting of the amino acids G, E, R, A, and V;
X₁₃ is chosen from the group consisting of the amino acids R, and Q;
X₁₄ is chosen from the group consisting of the amino acids L and W;
X₁₅ is chosen from the group consisting of the amino acids W and R;
X₁₆ is chosen from the group consisting of the amino acids G, R, E, A, V, and W;
X₁₇ is chosen from the group consisting of the amino acids L, F, M, W, and Y;
X₁₈ is chosen from the group consisting of the amino acids V and I;
X₁₉ is chosen from the group consisting of the amino acids R, L, Q, and H;
wherein the formula may encompass conservative amino acid modifications at any position;
wherein, optionally, 1 to 10 amino acids are inserted or deleted;
wherein the amino acids between Z₁ and Z₂ are D type optical isomers;
and further wherein
Zᵢ is absent or is a peptide of length 1 to 25 composed of any amino acids;
Z₂ is absent or is a peptide of length 1 to 25 composed of any amino acids;
wherein one or more optional polyoxyalkelene spacer moieties are covalently bound to the peptide or mimetic thereof, to one or both of the strings Zᵢ and Z₂, or to a functional combination thereof; and,
F₁ and F₂, are each independently absent or are independently chosen chemical groups covalently bound to the peptide or mimetic thereof, to one or both of the strings Z₁ and Z₂, to the one or more polyoxyalkelene moieties, or to a functional combination thereof; and,
wherein the chemical groups are chosen independently from the group comprising NH₂, —N-biotinyl-K—CO—NH₂, wherein K is the D or L type optical isomer of Lysine, and —NH-(PEG)ₙ-COOH, wherein n is any integer from 1 to 10,000, and a detectable label.

3. The isolated peptide or mimetic of claim 2 wherein the peptide or mimetic specifically binds to VEGF.

4. The isolated peptide or mimetic of claim 2 wherein the polyoxyalkelene is polyetheleneglycol.

5. The isolated peptide or mimetic of claim 4 wherein the polyetheleneglycol spacer has the structure —NH-PEG₂-CO—.

6. The isolated peptide or mimetic of claim 2 wherein (SEQ ID NO: 35)
X₁ is N;
X₂ is T;
X₃ is H;
X₄ is W;
X₅ is A;
X₆ is S;
X₇ is D;
X₈ is I;
X₉ is R;
X₁₀ is T;
X₁₁ is P;
X₁₂ is G;
X₁₃ is Q;
X₁₄ is L;
X₁₅ is W;
X₁₆ is G;
X₁₇ is L;
X₁₈ is V;
X₁₉ is R;
and conservative amino acid modifications at any position within the peptide.

7. An isolated peptide or mimetic thereof which is at least 90% identical to the isolated peptide or mimetic of claim 6.

8. The isolated peptide or mimetic of claim 2 wherein (SEQ ID NO: 36)
X₁ is N;
X₂ is A;
X₃ is H;
X₄ is W;
X₅ is A;
X₆ is S;
X₇ is N;
X₈ is I;
X₉ is R;
X₁₀ is T;
X₁₁ is P;
X₁₂ is G;
X₁₃ is Q;
X₁₄ is L;
X₁₅ is W;
X₁₆ is R;
X₁₇ is L;
X₁₈ is V;
X₁₉ is R;
and conservative amino acid modifications at any position within the peptide.

9. An isolated peptide or mimetic thereof which is at least 90% identical to the isolated peptide or mimetic of claim 8.

10. The isolated peptide or mimetic of claim 2 wherein (SEQ ID NO: 37)
X₁ is N;
X₂ is A;
X₃ is H;
X₄ is W;
X₅ is A;
X₆ is S;
X₇ is N;
X₈ is I;
X₉ is R;
X₁₀ is chosen from the group consisting of the amino acids T and S;
X₁₁ is P;
X₁₂ is G;
X₁₃ is chosen from the group consisting of the amino acids R, and Q;
X₁₄ is L;
X₁₅ is W;
X₁₆ is chosen from the group consisting of the amino acids G, R, E;
X₁₇ is L;
X₁₈ is V;
X₁₉ is R;
and conservative amino acid modifications at any position within the peptide.

11. An isolated peptide or mimetic thereof which is at least 90% identical to the isolated peptide or mimetic of claim 10.

12. The isolated peptide or mimetic of claim 10 wherein (SEQ ID NO: 38)
X₁₀ is S;
X₁₃ is R;
X₁₆ is G;
and conservative amino acid modifications at any position within the peptide.

13. An isolated peptide or mimetic thereof which is at least 90% identical to the isolated peptide or mimetic of claim 12.

14. The isolated peptide or mimetic of claim 10 wherein (SEQ ID NO: 39)
X₁₀ is T;
X₁₃ is R;
X₁₆ is G;
and conservative amino acid modifications at any position within the peptide.

15. An isolated peptide or mimetic thereof which is at least 90% identical to the isolated peptide or mimetic of claim 14.

16. The isolated peptide or mimetic of claim 10 wherein (SEQ ID NO: 40)
X₁₀ is T;
X₁₃ is Q;
X₁₆ is G;
and conservative amino acid modifications at any position within the peptide.

17. An isolated peptide or mimetic thereof which is at least 90% identical to the isolated peptide or mimetic of claim 16.

18. The isolated peptide or mimetic of claim 10 wherein (SEQ ID NO: 41)
X₁₀ is T;
X₁₃ is R;
X₁₆ is R;
and conservative amino acid modifications at any position within the peptide.

19. An isolated peptide or mimetic thereof which is at least 90% identical to the isolated peptide or mimetic of claim 18.

20. The isolated peptide or mimetic of claim 10 wherein (SEQ ID NO: 42)
X₁₀ is T;
X₁₃ is R;
X₁₆ is E;
and conservative amino acid modifications at any position within the peptide.

21. An isolated peptide or mimetic thereof which is at least 90% identical to the isolated peptide or mimetic of claim 20.

22. An isolated peptide or mimetic thereof, the peptide being composed of D-type optical isomers and comprising the amino acid sequence
F₁-Z₁-VQEDVSSTLGSWVLLPFHRGTRLSVWVT-Z₂-F₂
or the amino acid sequence
F₁-Z₁-GGFEGLSQARKDQLWLFLMQHIRSYRTIT-Z₂-F₂
wherein
Z₁ is absent or is a peptide of length 1 to 25 composed of any amino acids;
Z₂ is absent or is a peptide of length 1 to 25 composed of any amino acids;
wherein one or more optional polyoxyalkelene spacer moieties are covalently bound to the peptide or mimetic thereof, to one or both of the strings Z₁ and Z₂, or to a functional combination thereof; and,
F₁ and F₂, are each independently absent or are independently chosen chemical groups covalently bound to the peptide or mimetic thereof, to one or both of the strings Z₁ and Z₂, to the one or more polyoxyalkelene moieties, or to a functional combination thereof; and,
wherein the chemical groups are chosen independently from the group comprising NH₂, —N-biotinyl-K—CO—NH₂, wherein K is the D or L type optical isomer of Lysine, —NH-(PEG)ₙ-COOH, wherein n is any integer from 1 to 10,000, and a detectable label.

23. An isolated peptide or mimetic thereof which binds to VEGF, said peptide comprising a formula (SEQ ID NO: 43):
F₁-Z₁-S-X₁-T-L-X₂-S-X₃-V-X₅-Z₂-F₂
X₁ is any amino acid;
X₂ is any amino acid;
X₃ is chosen from the group consisting of the amino acids W and F;
X₅ is chosen from the group consisting of the amino acids L and I;
wherein the formula may encompass conservative amino acid modifications at any position;
wherein, optionally, 1 to 10 amino acids are inserted or deleted;
wherein the amino acids between Z₁ and Z₂ are D type optical isomers;
and further wherein
Z₁ is absent or is a peptide of length 1 to 25 composed of any amino acids;
Z₂ is absent or is a peptide of length 1 to 25 composed of any amino acids;
wherein one or more optional polyoxyalkelene spacer moieties are covalently bound to the peptide or mimetic thereof, to one or both of the strings Z₁ and Z₂, or to a functional combination thereof; and,
F₁ and F₂, are each independently absent or are independently chosen chemical groups covalently bound to the peptide or mimetic thereof, to one or both of the strings Z₁ and Z₂, to the one or more polyoxyalkelene moieties, or to a functional combination thereof; and,
wherein the chemical groups are chosen independently from the group comprising —NH₂, —N-biotinyl-K—CO—NH₂, wherein K is the D or L type optical isomer of Lysine, —NH-(PEG)ₙ-COOH, wherein n is any integer from 1 to 10,000, and a detectable label.

24. The isolated peptide or mimetic thereof of claim 23, wherein (SEQ ID NO: 44)
X₁ is S;
X₂ is G;
X₃ is W; and,
X₅ is L;

25. The isolated peptide or mimetic thereof of claim 24, wherein (SEQ ID NO: 45)
Z₁ comprises GVQEDV; and,
Z₂ comprises LPFHRGTRLSVWVT

26. The isolated peptide or mimetic thereof of claim 23, wherein (SEQ ID NO: 46)
X₁ is P;
X₂ is S;
X₃ is F; and
X₅ is I

27. The isolated peptide or mimetic thereof of claim 26, wherein (SEQ ID NO: 47)
Z₁ comprises GAGLWWGFCTDQHCIFK; and
Z₂ comprises T.

28. The isolated peptide or mimetic of claim 2, wherein Z₁ comprises GSGS (SEQ ID NO: 2) or SGSSSGSGS (SEQ ID NO: 3).

29. (canceled)

30. The isolated peptide or mimetic of claim 2 wherein the polyoxyalkelene spacer moiety is —NH-PEGₙ-CO—; and
wherein n is an integer between 1 and 100.

31. The isolated peptide or mimetic of claim 30 wherein the amino acids are D type optical isomers.

32. The isolated peptide or mimetic of claim 30 wherein said peptide is covalently bound to the chemical group F₁ is
—N-biotinyl-K—CO—NH₂, wherein K is the D or L type optical isomer of Lysine;

33. The isolated peptide or mimetic of claim 30 wherein the chemical group F₁ is
H₂N-PEGₓ-CO—
and wherein x is an integer between 1 and 10,000.

34. The isolated peptide or mimetic of claim 30 wherein
the chemical group F₂ is —NH₂.

35. The isolated peptide or mimetic of claim 30 wherein the chemical group F₁ is H₂N-PEG₅₀₀₀-CO—,
and the polyoxyalkelene spacer moiety is
—NH-PEG₂-CO—.

36. An isolated peptide or mimetic thereof, the peptide or mimetic being composed of D-type optical isomers and comprising an amino acid sequence selected from the group consisting of:
(i) the amino acid sequence NALHWVCASNICWRSPWAGRLWGLVRL (G2306);
(ii) the amino acid sequence GNALHWVCASNICWRTPWAGQLWRLVRL; and
(iii) an amino acid sequence at least 70% identical to the sequence GNALHWVCASNICWRTPWAGQLWRLVRL.

37. (canceled)

38. (canceled)

39. An isolated peptide or mimetic thereof which specifically binds to VEGF wherein the amino acid sequence is chosen from the group consisting of
(SEQ ID NO: 11)
GNALHWVCASNICWRSPWAGRLWGLVRLT;
(SEQ ID NO: 12)
SGSSSGSGSGNTLHWVCASDICWRTPWAGQLWGLVRLT;
(SEQ ID NO: 13)
NTLHWVCASDICWRTPWAGQLWGLVRLT;
(SEQ ID NO: 14)
SGSSSGSGSGNTLHWVCASDICWRTPWAGQLWGLVRL;
(SEQ ID NO: 15)
NTLHWVCASDICWRTPWAGQLWGLVRL;
(SEQ ID NO: 16)
SGSSSGSGSGNALHWVCASNICWRTPWAGQLWRLVRLT;
(SEQ ID NO: 17)
NALHWVCASNICWRTPWAGQLWRLVRL;
(SEQ ID NO: 18)
NALHWVCASNICWRTPWAGQLWRLVRLT;
(SEQ ID NO: 19)
SGSSSGSGSGNALHWVCASNICWRTPWAGQLWRLVRL;
(SEQ ID NO: 20)
NALHWVCASNICWRSPWAGRLWGLVRL;
(SEQ ID NO: 21)
NALHWVCASNICWRSPWAGRLWGLVRL;
(SEQ ID NO: 22)
NALHWVCASNICWRTPWAGRLWGLVRL;
(SEQ ID NO: 23)
NALHWVCASNICWRTPWAGQLWGLVRL;
(SEQ ID NO: 24)
NALHWVCASNICWRTPWAGRLWRLVRL;
(SEQ ID NO: 25)
NALHWVCASNICWRTPWAGRLWELVRL;
(SEQ ID NO: 26)
VQEDVSSTLGSWVLLPFHRGTRLSVWVT;
(SEQ ID NO: 27)
GGFEGLSQARKDQLWLFLMQHIRSYRTIT;
(SEQ ID NO: 28)
GVQEDVSSTLGSWVLLPFHRGTRLSVWVT;
(SEQ ID NO: 29)
GAGLWWGFCTDQHCIFKSPTLSSFVIVDT;
(SEQ ID NO: 26)
GGFEGLSQARKDQLWLFLMQHIRSYRTIT;
(SEQ ID NO: 30)
GNALHWVCASNICWRPPWAGRLWGLVRLT;
the sequences shown in FIG. 11 and FIG. 13;
and fragments thereof,
and conservative amino acid modifications at any position within the peptide,
and wherein the peptide is composed of D type optical isomers.

40. An isolated peptide or mimetic thereof which is at least 90% identical to the isolated peptide or mimetic of claim 39.

41. An isolated peptide or mimetic thereof which specifically binds to VEGF, the peptide being composed of D type optical isomers and wherein the peptide's amino acid sequence is selected from the group comprising
GNALHWVCASNICWRTPWAGQLWRLVRL, and NALHWVOASNICWRSPWACRLWGLVRL
or variants thereof having conservative amino acid additions, deletions, or substitutions,
wherein the variants contain between 4 and 90 amino acids;
wherein the variants are at least 70% identical to at least one of SEQ ID NO X-Y; and
wherein the variants consist of D type optical isomers.

42. The isolated peptide or mimetic of claim 36 further comprising a chemical group attached to the N-terminal amino acid, the chemical group having the structure H₂N-PEGx-CO— wherein x is an integer between 1 and 10,000;
further comprising a chemical group attached the C-terminal amino acid, the chemical group having the structure NH-PEGₙ-CO—NH₂, wherein n is an integer from 1 to 100.

43. The isolated peptide or mimetic of claim 42, wherein x is 2 and n is 5000.

44. A pharmaceutical composition comprising the isolated peptide or mimetic of claim 1 and a pharmaceutically acceptable carrier.

45. The isolated peptide or mimetic thereof of claim 1, wherein the isolated peptide or mimetic thereof, specifically binds to VEGF with a KD selected from the group consisting of 1×10⁻⁶ M or less.

46. The isolated peptide or mimetic thereof of claim 1, wherein the isolated peptide or mimetic thereof is a cyclic peptide.

47. The isolated peptide or mimetic thereof of claim 1, wherein the isolated peptide or mimetic thereof contains an intramolecular disulfide bond.

48. A method of treating a VEGF modulated disease in a subject, comprising administering to the subject an effective amount isolated peptide or mimetic of claim 1, thereby treating the VEGF modulated disease.

49.-51. (canceled)

52. A method for detecting VEGF in a biological sample, comprising:
(a) incubating a biological sample with a peptide or mimetic thereof which specifically binds to VEGF wherein the amino acids in said peptide or mimetic thereof are D type optical isomers and wherein said incubation allows the formation of a complex between VEGF and said peptide or mimetic thereof; and
(b) detecting VEGF bound to the immobilized capture reagent.

53.-56. (canceled)


SEQUENCE LISTING (129 sequences)

SEQ ID NO: 1 - protein, 78 aa, Artificial Sequence
  Description of Artificial Sequence: Synthetic polypeptide
  MOD_RES (1)..(25): Any amino acid or absent
  MOD_RES (27)..(27): Asn, Tyr, Phe, Asp, Ile or His
  MOD_RES (28)..(28): Ala, Thr or Val
  MOD_RES (30)..(30): His, Gln or Arg
  MOD_RES (31)..(31): Trp or Arg
  MOD_RES (34)..(34): Ala or Val
  MOD_RES (35)..(35): Ser or Leu
  MOD_RES (36)..(36): Asn, Ser or Asp
  MOD_RES (37)..(37): Ile, Val or His
  MOD_RES (40)..(40): Arg or Met
  MOD_RES (41)..(41): Ser, Thr, Pro or Phe
  MOD_RES (42)..(42): Pro or Leu
  MOD_RES (45)..(45): Gly, Glu, Arg, Ala or Val
  MOD_RES (46)..(46): Arg or Gln
  MOD_RES (47)..(47): Leu or Trp
  MOD_RES (48)..(48): Trp or Arg
  MOD_RES (49)..(49): Gly, Arg, Glu, Ala, Val or Trp
  MOD_RES (50)..(50): Leu, Phe, Met, Trp or Tyr
  MOD_RES (51)..(51): Val or Ile
  MOD_RES (52)..(52): Arg, Leu, Gln or His
  MOD_RES (54)..(78): Any amino acid or absent
  see specification as filed for detailed description of substitutions and preferred embodiments
XXXXXXXXXXXXXXXXXXXXXXXXXGXXLXXVCXXXXCWXXXWAXXXXXXXXLXXXXXXXXXXXXXXXXXXXXXXXXX

SEQ ID NO: 2 - protein, 4 aa, Artificial Sequence
  Description of Artificial Sequence: Synthetic peptide
GSGS

SEQ ID NO: 3 - protein, 9 aa, Artificial Sequence
  Description of Artificial Sequence: Synthetic peptide
SGSSSGSGS

SEQ ID NO: 4 - protein, 9 aa, Artificial Sequence
  Description of Artificial Sequence: Synthetic peptide
TSGGSSGSS

SEQ ID NO: 5 - protein, 16 aa, Artificial Sequence
  Description of Artificial Sequence: Synthetic peptide
TSGGSSGSSLGVASAI

SEQ ID NO: 6 - protein, 17 aa, Artificial Sequence
  Description of Artificial Sequence: Synthetic peptide
MHHHHHHSGSSSGSGSG

SEQ ID NO: 7 - protein, 10 aa, Artificial Sequence
  Description of Artificial Sequence: Synthetic peptide
SGRSSGSGSG

SEQ ID NO: 8 - protein, 16 aa, Artificial Sequence
  Description of Artificial Sequence: Synthetic peptide
TSGGSSGSSLVQHPLF

SEQ ID NO: 9 - protein, 10 aa, Artificial Sequence
  Description of Artificial Sequence: Synthetic peptide
SGSSSGSGFR

SEQ ID NO: 10 - protein, 10 aa, Artificial Sequence
  Description of Artificial Sequence: Synthetic peptide
SDSSSGSGSG

SEQ ID NO: 11 - protein, 29 aa, Artificial Sequence
  Description of Artificial Sequence: Synthetic peptide
GNALHWVCASNICWRSPWAGRLWGLVRLT

SEQ ID NO: 12 - protein, 38 aa, Artificial Sequence
  Description of Artificial Sequence: Synthetic polypeptide
SGSSSGSGSGNTLHWVCASDICWRTPWAGQLWGLVRLT

SEQ ID NO: 13 - protein, 28 aa, Artificial Sequence
  Description of Artificial Sequence: Synthetic peptide
NTLHWVCASDICWRTPWAGQLWGLVRLT

SEQ ID NO: 14 - protein, 37 aa, Artificial Sequence
  Description of Artificial Sequence: Synthetic polypeptide
SGSSSGSGSGNTLHWVCASDICWRTPWAGQLWGLVRL

SEQ ID NO: 15 - protein, 27 aa, Artificial Sequence
  Description of Artificial Sequence: Synthetic peptide
NTLHWVCASDICWRTPWAGQLWGLVRL

SEQ ID NO: 16 - protein, 38 aa, Artificial Sequence
  Description of Artificial Sequence: Synthetic polypeptide
SGSSSGSGSGNALHWVCASNICWRTPWAGQLWRLVRLT

SEQ ID NO: 17 - protein, 27 aa, Artificial Sequence
  Description of Artificial Sequence: Synthetic peptide
NALHWVCASNICWRTPWAGQLWRLVRL

SEQ ID NO: 18 - protein, 28 aa, Artificial Sequence
  Description of Artificial Sequence: Synthetic peptide
NALHWVCASNICWRTPWAGQLWRLVRLT

SEQ ID NO: 19 - protein, 37 aa, Artificial Sequence
  Description of Artificial Sequence: Synthetic polypeptide
SGSSSGSGSGNALHWVCASNICWRTPWAGQLWRLVRL

SEQ ID NO: 20 - protein, 27 aa, Artificial Sequence
  Description of Artificial Sequence: Synthetic peptide
NALHWVCASNICWRSPWAGRLWGLVRL

SEQ ID NO: 21 - protein, 27 aa, Artificial Sequence
  Description of Artificial Sequence: Synthetic peptide
NALHWVCASNICWRTPWAGRLWGLVRL

SEQ ID NO: 22 - protein, 27 aa, Artificial Sequence
  Description of Artificial Sequence: Synthetic peptide
NALHWVCASNICWRTPWAGQLWGLVRL

SEQ ID NO: 23 - protein, 27 aa, Artificial Sequence
  Description of Artificial Sequence: Synthetic peptide
NALHWVCASNICWRTPWAGRLWRLVRL

SEQ ID NO: 24 - protein, 27 aa, Artificial Sequence
  Description of Artificial Sequence: Synthetic peptide
NALHWVCASNICWRTPWAGRLWELVRL

SEQ ID NO: 25 - protein, 28 aa, Artificial Sequence
  Description of Artificial Sequence: Synthetic peptide
VQEDVSSTLGSWVLLPFHRGTRLSVWVT

SEQ ID NO: 26 - protein, 29 aa, Artificial Sequence
  Description of Artificial Sequence: Synthetic peptide
GGFEGLSQARKDQLWLFLMQHIRSYRTIT

SEQ ID NO: 27 - protein, 9 aa, Artificial Sequence
  Description of Artificial Sequence: Synthetic peptide
  MOD_RES (2)..(2): Any amino acid
  MOD_RES (5)..(5): Any amino acid
  MOD_RES (7)..(7): Trp or Phe
  MOD_RES (9)..(9): Leu or Ile
  see specification as filed for detailed description of substitutions and preferred embodiments
SXTLXSXVX

SEQ ID NO: 28 - protein, 29 aa, Artificial Sequence
  Description of Artificial Sequence: Synthetic peptide
GVQEDVSSTLGSWVLLPFHRGTRLSVWVT

SEQ ID NO: 29 - protein, 29 aa, Artificial Sequence
  Description of Artificial Sequence: Synthetic peptide
GAGLWWGFCTDQHCIFKSPTLSSFVIVDT

SEQ ID NO: 30 - protein, 29 aa, Artificial Sequence
  Description of Artificial Sequence: Synthetic peptide
GNALHWVCASNICWRPPWAGRLWGLVRLT

SEQ ID NO: 31 - protein, 28 aa, Artificial Sequence
  Description of Artificial Sequence: Synthetic peptide
  MOD_RES (2)..(2): Asn, Tyr, Phe, Asp, Ile or His
  MOD_RES (3)..(3): Ala, Thr or Val
  MOD_RES (5)..(5): His, Gln or Arg
  MOD_RES (6)..(6): Trp or Arg
  MOD_RES (9)..(9): Ala or Val
  MOD_RES (10)..(10): Ser or Leu
  MOD_RES (11)..(11): Asn, Ser or Asp
  MOD_RES (12)..(12): Ile, Val or His
  MOD_RES (15)..(15): Arg or Met
  MOD_RES (16)..(16): Ser, Thr, Pro or Phe
  MOD_RES (17)..(17): Pro of Leu
  MOD_RES (20)..(20): Gly, Glu, Arg, Ala or Val
  MOD_RES (21)..(21): Arg or Gln
  MOD_RES (22)..(22): Leu or Trp
  MOD_RES (23)..(23): Trp or Arg
  MOD_RES (24)..(24): Gly, Arg, Glu, Ala, Val or Trp
  MOD_RES (25)..(25): Leu, Phe, Met, Trp or Tyr
  MOD_RES (26)..(26): Val or Ile
  MOD_RES (27)..(27): Arg, Leu, Gln or His
  see specification as filed for detailed description of substitutions and preferred embodiments
GXXLXXVCXXXXCWXXXWAXXXXXXXXL

SEQ ID NO: 32 - protein, 43 aa, Artificial Sequence
  Description of Artificial Sequence: Synthetic polypeptide
GQNHHEVVKFMDVYQRSYCHPIETLVDIFQEYPDEIEYIFKPS

SEQ ID NO: 33 - protein, 59 aa, Artificial Sequence
  Description of Artificial Sequence: Synthetic polypeptide
CVPLMRCGGCCNDEGLECVPTEESNITMQIMRIKPHQGQHIGEMSFLQHNKCECRPKKD

SEQ ID NO: 34 - protein, 78 aa, Artificial Sequence; identical to SEQ ID NO: 1
  Description of Artificial Sequence: Synthetic polypeptide
  MOD_RES (1)..(25): Any amino acid or absent
  MOD_RES (27)..(27): Asn, Tyr, Phe, Asp, Ile or His
  MOD_RES (28)..(28): Ala, Thr or Val
  MOD_RES (30)..(30): His, Gln or Arg
  MOD_RES (31)..(31): Trp or Arg
  MOD_RES (34)..(34): Ala or Val
  MOD_RES (35)..(35): Ser or Leu
  MOD_RES (36)..(36): Asn, Ser or Asp
  MOD_RES (37)..(37): Ile, Val or His
  MOD_RES (40)..(40): Arg or Met
  MOD_RES (41)..(41): Ser, Thr, Pro or Phe
  MOD_RES (42)..(42): Pro or Leu
  MOD_RES (45)..(45): Gly, Gly, Arg, Ala or Val
  MOD_RES (46)..(46): Arg or Gln
  MOD_RES (47)..(47): Leu or Trp
  MOD_RES (48)..(48): Trp or Arg
  MOD_RES (49)..(49): Gly, Arg, Glu, Ala, Val or Trp
  MOD_RES (50)..(50): Leu, Phe, Met, Trp or Tyr
  MOD_RES (51)..(51): Val or Ile
  MOD_RES (52)..(52): Arg, Leu, Gln or His
  MOD_RES (54)..(78): Any amino acid or absent
  see specification as filed for detailed description of substitutions and preferred embodiments

SEQ ID NO: 35 - protein, 78 aa, Artificial Sequence
  Description of Artificial Sequence: Synthetic polypeptide
  MOD_RES (1)..(25): Any amino acid or absent
  MOD_RES (54)..(78): Any amino acid or absent
XXXXXXXXXXXXXXXXXXXXXXXXXGNTLHWVCASDICWRTPWAGQLWGLVRLXXXXXXXXXXXXXXXXXXXXXXXXX

SEQ ID NO: 36 - protein, 78 aa, Artificial Sequence
  Description of Artificial Sequence: Synthetic polypeptide
  MOD_RES (1)..(25): Any amino acid or absent
  MOD_RES (54)..(78): Any amino acid or absent
XXXXXXXXXXXXXXXXXXXXXXXXXGNALHWVCASNICWRTPWAGQLWRLVRLXXXXXXXXXXXXXXXXXXXXXXXXX

SEQ ID NO: 37 - protein, 78 aa, Artificial Sequence
  Description of Artificial Sequence: Synthetic polypeptide
  MOD_RES (1)..(25): Any amino acid or absent
  MOD_RES (41)..(41): Thr or Ser
  MOD_RES (46)..(46): Arg or Gln
  MOD_RES (49)..(49): Gly, Arg or Glu
  MOD_RES (54)..(78): Any amino acid or absent
XXXXXXXXXXXXXXXXXXXXXXXXXGNALHWVCASNICWRXPWAGXLWXLVRLXXXXXXXXXXXXXXXXXXXXXXXXX

SEQ ID NO: 38 - protein, 78 aa, Artificial Sequence
  Description of Artificial Sequence: Synthetic polypeptide
  MOD_RES (1)..(25): Any amino acid or absent
  MOD_RES (54)..(78): Any amino acid or absent
XXXXXXXXXXXXXXXXXXXXXXXXXGNALHWVCASNICWRSPWAGRLWGLVRLXXXXXXXXXXXXXXXXXXXXXXXXX

SEQ ID NO: 39 - protein, 78 aa, Artificial Sequence
  Description of Artificial Sequence: Synthetic polypeptide
  MOD_RES (1)..(25): Any amino acid or absent
  MOD_RES (54)..(78): Any amino acid or absent
XXXXXXXXXXXXXXXXXXXXXXXXXGNALHWVCASNICWRTPWAGRLWGLVRLXXXXXXXXXXXXXXXXXXXXXXXXX

SEQ ID NO: 40 - protein, 78 aa, Artificial Sequence
  Description of Artificial Sequence: Synthetic polypeptide
  MOD_RES (1)..(25): Any amino acid or absent
  MOD_RES (54)..(78): Any amino acid or absent
XXXXXXXXXXXXXXXXXXXXXXXXXGNALHWVCASNICWRTPWAGQLWGLVRLXXXXXXXXXXXXXXXXXXXXXXXXX

SEQ ID NO: 41 - protein, 78 aa, Artificial Sequence
  Description of Artificial Sequence: Synthetic polypeptide
  MOD_RES (1)..(25): Any amino acid or absent
  MOD_RES (54)..(78): Any amino acid or absent
XXXXXXXXXXXXXXXXXXXXXXXXXGNALHWVCASNICWRTPWAGRLWRLVRLXXXXXXXXXXXXXXXXXXXXXXXXX

SEQ ID NO: 42 - protein, 78 aa, Artificial Sequence
  Description of Artificial Sequence: Synthetic polypeptide
  MOD_RES (1)..(25): Xaa can be any naturally occurring amino acid
  MOD_RES (54)..(78): Xaa can be any naturally occurring amino acid
XXXXXXXXXXXXXXXXXXXXXXXXXGNALHWVCASNICWRTPWAGRLWELVRLXXXXXXXXXXXXXXXXXXXXXXXXX

SEQ ID NO: 43 - protein, 59 aa, Artificial Sequence
  Description of Artificial Sequence: Synthetic polypeptide
  MOD_RES (1)..(25): Any amino acid or absent
  MOD_RES (27)..(27): Any amino acid
  MOD_RES (30)..(30): Any amino acid
  MOD_RES (32)..(32): Trp or Phe
  MOD_RES (34)..(34): Leu or Ile
  MOD_RES (35)..(59): Any amino acid or absent
  see specification as filed for detailed description of substitutions and preferred embodiments
XXXXXXXXXXXXXXXXXXXXXXXXXSXTLXSXVXXXXXXXXXXXXXXXXXXXXXXXXXX

SEQ ID NO: 44 - protein, 59 aa, Artificial Sequence
  Description of Artificial Sequence: Synthetic polypeptide
  MOD_RES (1)..(25): Any amino acid or absent
  MOD_RES (35)..(59): Any amino acid or absent
XXXXXXXXXXXXXXXXXXXXXXXXXSSTLGSWVLXXXXXXXXXXXXXXXXXXXXXXXXX

SEQ ID NO: 45 - protein, 29 aa, Artificial Sequence
  Description of Artificial Sequence: Synthetic peptide
GVQEDVSSTLGSWVLLPFHRGTRLSVWVT

SEQ ID NO: 46 - protein, 59 aa, Artificial Sequence
  Description of Artificial Sequence: Synthetic polypeptide
  MOD_RES (1)..(25): Any amino acid or absent
  MOD_RES (35)..(59): Any amino acid or absent
XXXXXXXXXXXXXXXXXXXXXXXXXSPTLSSFVIXXXXXXXXXXXXXXXXXXXXXXXXX

SEQ ID NO: 47 - protein, 27 aa, Artificial Sequence
  Description of Artificial Sequence: Synthetic peptide
GAGLWWGFCTDQHCIFKSPTLSSFVIT

SEQ ID NO: 48 - protein, 60 aa, Artificial Sequence
  Description of Artificial Sequence: Synthetic polypeptide
  MOD_RES (18)..(44): Any amino acid
MHHHHHHSGSSSGSGSGXXXXXXXXXXXXXXXXXXXXXXXXXXXTSGGSSGSSLGVASAI

SEQ ID NO: 49 - protein, 27 aa, Artificial Sequence
  Description of Artificial Sequence: Synthetic peptide
NALHWVCASNICWRTPWAEQLWGLVRL

SEQ ID NO: 50 - protein, 27 aa, Artificial Sequence
  Description of Artificial Sequence: Synthetic peptide
NALHWVCASNICWRTPWAGQLWGLIRL

SEQ ID NO: 51 - protein, 27 aa, Artificial Sequence
  Description of Artificial Sequence: Synthetic peptide
NTLHWVCASNICWRSPWAGQLWGLVRL

SEQ ID NO: 52 - protein, 27 aa, Artificial Sequence
  Description of Artificial Sequence: Synthetic peptide
NALHWVCASNHCWRSPWAGRLWELVRL

SEQ ID NO: 53 - protein, 27 aa, Artificial Sequence
  Description of Artificial Sequence: Synthetic peptide
NALHWVCASNICWRTPWAGRLWGLIRL

SEQ ID NO: 54 - protein, 27 aa, Artificial Sequence
  Description of Artificial Sequence: Synthetic peptide
NTLHWVCASNICWRTPWAGRLWRLVRL

SEQ ID NO: 55 - protein, 27 aa, Artificial Sequence
  Description of Artificial Sequence: Synthetic peptide
NALHWVCASNICWRSPWAGRLWGYVRL

SEQ ID NO: 56 - protein, 27 aa, Artificial Sequence
  Description of Artificial Sequence: Synthetic peptide
NALHWVCASNICWRSPWAGQLWGLVRL

SEQ ID NO: 57 - protein, 27 aa, Artificial Sequence
  Description of Artificial Sequence: Synthetic peptide
NALHWVCASNICWRSPWAGQLWGLIRL

SEQ ID NO: 58 - protein, 27 aa, Artificial Sequence
  Description of Artificial Sequence: Synthetic peptide
NALHWVCASNICWRSPWAGQLWRLIRL

SEQ ID NO: 59 - protein, 27 aa, Artificial Sequence
  Description of Artificial Sequence: Synthetic peptide
NALHWVCASNICWRSPWAEQLWGLVRL

SEQ ID NO: 60 - protein, 27 aa, Artificial Sequence
  Description of Artificial Sequence: Synthetic peptide
YALHWVCALNICWRTPWAGQLWGLIRL

SEQ ID NO: 61 - protein, 27 aa, Artificial Sequence
  Description of Artificial Sequence: Synthetic peptide
NALHWVCASNICWRSPWARQLWRLVQL

SEQ ID NO: 62 - protein, 27 aa, Artificial Sequence
  Description of Artificial Sequence: Synthetic peptide
NALHWVCASNICWRSPWARQLWGFVRL

SEQ ID NO: 63 - protein, 27 aa, Artificial Sequence
  Description of Artificial Sequence: Synthetic peptide
NTLHWVCASNICWRTPWAGRLWGLIRL

SEQ ID NO: 64 - protein, 27 aa, Artificial Sequence
  Description of Artificial Sequence: Synthetic peptide
NALHWVCASNICWRSPWAGQLRWFVRL

SEQ ID NO: 65 - protein, 27 aa, Artificial Sequence
  Description of Artificial Sequence: Synthetic peptide
NALHWVCASNICWRTPWAGRLWWLVRL

SEQ ID NO: 66 - protein, 26 aa, Artificial Sequence
  Description of Artificial Sequence: Synthetic peptide
NALHWVCANICWRTPWAGRLWRLVRL

SEQ ID NO: 67 - protein, 26 aa, Artificial Sequence
  Description of Artificial Sequence: Synthetic peptide
YALHWVCANICWRTLWAGQLWGLVRL

SEQ ID NO: 68 - protein, 27 aa, Artificial Sequence
  Description of Artificial Sequence: Synthetic peptide
NALHWVCASDICWRSPWAGRLWGLVRL

SEQ ID NO: 69 - protein, 27 aa, Artificial Sequence
  Description of Artificial Sequence: Synthetic peptide
FALHWVCASNICWRTPWAGRLWRLVRL

SEQ ID NO: 70 - protein, 27 aa, Artificial Sequence
  Description of Artificial Sequence: Synthetic peptide
NALHWVCASNVCWRTPWAGRLWGFVRL

SEQ ID NO: 71 - protein, 27 aa, Artificial Sequence
  Description of Artificial Sequence: Synthetic peptide
NALHWVCASNICWRTPWAERLWELVRL

SEQ ID NO: 72 - protein, 27 aa, Artificial Sequence
  Description of Artificial Sequence: Synthetic peptide
NALHWVCASNICWRSPWAGRLWGLIRL

SEQ ID NO: 73 - protein, 27 aa, Artificial Sequence
  Description of Artificial Sequence: Synthetic peptide
NALHWVCASNICWRSPWAGQLWGLIRL

SEQ ID NO: 74 - protein, 27 aa, Artificial Sequence
  Description of Artificial Sequence: Synthetic peptide
NALHWVCASNVCWRSPWAGRLWGLVRL

SEQ ID NO: 75 - protein, 27 aa, Artificial Sequence
  Description of Artificial Sequence: Synthetic peptide
NALHWVCASNICWRSPWAGRLWELVRL

SEQ ID NO: 76 - protein, 60 aa, Artificial Sequence
  Description of Artificial Sequence: Synthetic polypeptide
MHHHHHHSGSSSGSGSGNALHWVCASNICWRSPWAGRLWALIRLTSGGSSGSSLGVASAI

SEQ ID NO: 77 - protein, 60 aa, Artificial Sequence
  Description of Artificial Sequence: Synthetic polypeptide
MHHHHHHSGSSSGLGSGNALHWVCASNICWRSPWAEQLWELVRLTSGGSSGSSLGVASAI

SEQ ID NO: 78 - protein, 60 aa, Artificial Sequence
  Description of Artificial Sequence: Synthetic polypeptide
  MOD_RES (27)..(27): Any amino acid
MHHHHHHSGSSSRSGSGYTLHWVCASXICWRSPWAGQLWEMIHLTSGGSSGSSLGVASAI

SEQ ID NO: 79 - protein, 60 aa, Artificial Sequence
  Description of Artificial Sequence: Synthetic polypeptide
  MOD_RES (27)..(27): Any amino acid
MHHHHHHSGISSGRGSGNTLHWVCASXICWRSPWAGRLWRLIRLTSGGSSGSSLGVASAI

SEQ ID NO: 80 - protein, 61 aa, Artificial Sequence
  Description of Artificial Sequence: Synthetic polypeptide
MHHHHHHHSGGSSGSGSGNALQWVCASNVCWRSPWAERLWELVRLTSGGSSGSSLGVASAI

SEQ ID NO: 81 - protein, 60 aa, Artificial Sequence
  Description of Artificial Sequence: Synthetic polypeptide
MHRHHHHSGSSSGSGSGNALHWVCASNICWRSPWAGQLWRLVRLTSGGSSGSSLGVASAI

SEQ ID NO: 82 - protein, 60 aa, Artificial Sequence
  Description of Artificial Sequence: Synthetic polypeptide
MHHHHHHSGSSSGSGSGNVLHWVCASNVCWRSPWAAQLWGFVRLTSGGSSGSSLGVASAI

SEQ ID NO: 83 - protein, 60 aa, Artificial Sequence
  Description of Artificial Sequence: Synthetic polypeptide
MRHHLHHSSSSSGSGSGNALHWVCASNICWMTPWAGRLWGLILLTSGGSSGSSLGVASAI

SEQ ID NO: 84 - protein, 59 aa, Artificial Sequence
  Description of Artificial Sequence: Synthetic polypeptide
MHLHHHSGSSSGSGSGNALHWVCASNVCWRTPWAGQLWELVRLTSGGSSGSSLGVASAI

SEQ ID NO: 85 - protein, 60 aa, Artificial Sequence
  Description of Artificial Sequence: Synthetic polypeptide
MHHHHHHTGSSSGSGSGNALHWVCASNICWRTPWAEQLWELVRLTSGGSSGSSLGVASAI

SEQ ID NO: 86 - protein, 60 aa, Artificial Sequence
  Description of Artificial Sequence: Synthetic polypeptide
MHHHHLHSASSSGSGSGSALQWVCASNVCWRTPWAGRLWALVRLTSGGSSGSSLGVASAI

SEQ ID NO: 87 - protein, 59 aa, Artificial Sequence
  Description of Artificial Sequence: Synthetic polypeptide
MHHHHHSGSSSGSGSGNALHWVCASNICWRSPWAGQLWGLIRLTSGGSSGSSLGVASAI

SEQ ID NO: 88 - protein, 60 aa, Artificial Sequence
  Description of Artificial Sequence: Synthetic polypeptide
MHHHHHHSGSSSGSGSENALHWVCASNICWRSPWARQLWRLIRLTSGGSSGSSLGVASAI

SEQ ID NO: 89 - protein, 60 aa, Artificial Sequence
  Description of Artificial Sequence: Synthetic polypeptide
MHHHHHHSGSSSGSGSGNALHWVCASNICWRTPWAGQLWAWVRFTSGGSSGSSLGVASAI

SEQ ID NO: 90 - protein, 60 aa, Artificial Sequence
  Description of Artificial Sequence: Synthetic polypeptide
MHHHHHHSGSSSGSGSGNALHWVCASNICWRSPWAGRLWALIQLTSGGSSGSSLGVASAI

SEQ ID NO: 91 - protein, 60 aa, Artificial Sequence
  Description of Artificial Sequence: Synthetic polypeptide
  MOD_RES (27)..(27): Any amino acid
MHRHHHHSGSSAGSGSGNALQWVCASXICWRSPWAGQLWELVRLTSGGSSGSSLGVASAI

SEQ ID NO: 92 - protein, 59 aa, Artificial Sequence
  Description of Artificial Sequence: Synthetic polypeptide
MHHHHHSGSSSGSGSGNALHWVCASSICWRSPWAEQLWGLIRLTSGGSSGSSLGVASAI

SEQ ID NO: 93 - protein, 60 aa, Artificial Sequence
  Description of Artificial Sequence: Synthetic polypeptide
MYQHHHHSGSSSESGSENALHWVCASNICWRTPWAGRLWELVRLTSGGSSGSSLGVASAI

SEQ ID NO: 94 - protein, 60 aa, Artificial Sequence
  Description of Artificial Sequence: Synthetic polypeptide
MHHHHHHSGSSSRSGSGNALHWVCASNICWRSPWAGRLWALVRLTSGGSSGSSLGVASAI

SEQ ID NO: 95 - protein, 60 aa, Artificial Sequence
  Description of Artificial Sequence: Synthetic polypeptide
MHHHHHHSGSSSGSGSGNALHWVCASNICWRSPWAGQLWALIRLTSGGSSGSSLGVASAI

SEQ ID NO: 96 - protein, 60 aa, Artificial Sequence
  Description of Artificial Sequence: Synthetic polypeptide
  MOD_RES (30)..(30): Any amino acid
MHHHHHHSGSSSGSGSGNALHWVCASNICXRSPWAGQLWGLIRLTSGGSSGSSLGVASAI

SEQ ID NO: 97 - protein, 60 aa, Artificial Sequence
  Description of Artificial Sequence: Synthetic polypeptide
MHHHHHHSGSSSGSGSGNALHWVCASNICWRTPWAGQLWGLIRLTSGGSSGSSLGVASAI

SEQ ID NO: 98 - protein, 60 aa, Artificial Sequence
  Description of Artificial Sequence: Synthetic polypeptide
  MOD_RES (27)..(27): Any amino acid
MHHHHHHSGSSSGSGSGNTLHWVCASXICWRSPWAVQLWELVRLTSGGSSGSSLGVASAI

SEQ ID NO: 99 - protein, 60 aa, Artificial Sequence
  Description of Artificial Sequence: Synthetic polypeptide
  MOD_RES (27)..(27): Any amino acid
MHHHHHHSGSSSGSGSRNALHWVCASXICWRSPWAGQLWALIRLTSGVAAVLVLVHPLFN

SEQ ID NO: 100 - protein, 60 aa, Artificial Sequence
  Description of Artificial Sequence: Synthetic polypeptide
MHHHHHHSGSSSGSGSGNALHWVCASNICWRTPWAGQLWAFVRLTSGGSSGSSLGVASAI

SEQ ID NO: 101 - protein, 60 aa, Artificial Sequence
  Description of Artificial Sequence: Synthetic polypeptide
MHHHHHHSGSSSVSGSGNALQWVCASNVCWRSPWAGRLWWYVRLTSGGSSGSSLGVASAI

SEQ ID NO: 102 - protein, 60 aa, Artificial Sequence
  Description of Artificial Sequence: Synthetic polypeptide
MHHLHHHSGSSSGSGSGNALHWVCASNICWRSPWAGRLWELVRLTSGGSSGSSLGVASAI

SEQ ID NO: 103 - protein, 60 aa, Artificial Sequence
  Description of Artificial Sequence: Synthetic polypeptide
MHHHHHHSGSSSRSGSGNALHWVCASDICWRTPWAGRLWRLVRLTSGGSSGSSLGVASAI

SEQ ID NO: 104 - protein, 60 aa, Artificial Sequence
  Description of Artificial Sequence: Synthetic polypeptide
MHHHNHHSGRSSGSGSGNALHWVCASNVCWRSPWAGRLWGLVRLTSGGSSGSSLGVASAI

SEQ ID NO: 105 - protein, 60 aa, Artificial Sequence
  Description of Artificial Sequence: Synthetic polypeptide
MHHHHHHSGSSSGSGSGNTLHWVCASNVCWRSPWARQWWGFVRLTSGGSSGSSLGVASAI

SEQ ID NO: 106 - protein, 60 aa, Artificial Sequence
  Description of Artificial Sequence: Synthetic polypeptide
MHHHHHHSGSSSGSGSGNALHWVCASNICWRSPWAGRLWRLIRLTSGGSSGSSLGVASAI

SEQ ID NO: 107 - protein, 60 aa, Artificial Sequence
  Description of Artificial Sequence: Synthetic polypeptide
  MOD_RES (30)..(30): Xaa can be any naturally occurring amino acid
MHHHHHHPGSSSVSGSRYTLHWVCASNICXRSPWAGRLWRLIRLTSGGSSGSSLGVASAI

SEQ ID NO: 108 - protein, 60 aa, Artificial Sequence
  Description of Artificial Sequence: Synthetic polypeptide
MHHHHHHSGGSSGSGSRNALHWVCASNICWRTPWAGRLWRLIRLTSGGSSGSSLGVASAI

SEQ ID NO: 109 - protein, 60 aa, Artificial Sequence
  Description of Artificial Sequence: Synthetic polypeptide
MHNHHHHSGSSSGSESEDALHWVCASNICWRSPWAGRLWELVRLTSGGSSGSSLGVASAI

SEQ ID NO: 110 - protein, 60 aa, Artificial Sequence
  Description of Artificial Sequence: Synthetic polypeptide
MHHHHHHSGSSSGSGSGNALHWVCASNICWRSPWAGQLWGLVRLTSGGSSGSSLGVASAI

SEQ ID NO: 111 - protein, 60 aa, Artificial Sequence
  Description of Artificial Sequence: Synthetic polypeptide
  MOD_RES (18)..(18): Any amino acid
MHHHHHHSGSSSGSGPGXALHWVCASNICWRSPWAGRLWALVRLTSGGSSGSSLGVASAI

SEQ ID NO: 112 - protein, 60 aa, Artificial Sequence
  Description of Artificial Sequence: Synthetic polypeptide
MHHHHHHSGSSSGSGSGNALRWVCASNICWRTPWAGRLWRLVRLTSGGSSGSSLGVASAI

SEQ ID NO: 113 - protein, 60 aa, Artificial Sequence
  Description of Artificial Sequence: Synthetic polypeptide
MHHHHHHSGSSSGSGSGNALHWVCASNICWRTPWAGRLWGLIRLTSGGSSGSSLGVASAI

SEQ ID NO: 114 - protein, 61 aa, Artificial Sequence
  Description of Artificial Sequence: Synthetic polypeptide
MHHHHHHHSGSSSGSGSGYTLHWVCASNVCWRSPWAGQLWGVIRLTSGGSSGSSLGVASAI

SEQ ID NO: 115 - protein, 60 aa, Artificial Sequence
  Description of Artificial Sequence: Synthetic polypeptide
MHHHHHHSGSSSGSGYGYTLHWVCASNICWRSPWAGQLRRLVRLTSGGSSGSSLVQHPLF

SEQ ID NO: 116 - protein, 60 aa, Artificial Sequence
  Description of Artificial Sequence: Synthetic polypeptide
MHHHHHHSGSSSGSGSGNALHWVCASNICWRSPWAGQLWGLIRLTSGGSSGSSLGVASAI

SEQ ID NO: 117 - protein, 59 aa, Artificial Sequence
  Description of Artificial Sequence: Synthetic polypeptide
MHHHHHSGSSSGSGSGNALHWVCASNICWRTPWAGRLWRLVRLTSGGSSGSSLGVASAI

SEQ ID NO: 118 - protein, 59 aa, Artificial Sequence
  Description of Artificial Sequence: Synthetic polypeptide
MHRHHHSGSSSGSGSGNALHWVCASSICWRSLWAGRLWGLIRLTSGGSSGSSLGVASAI

SEQ ID NO: 119 - protein, 60 aa, Artificial Sequence
  Description of Artificial Sequence: Synthetic polypeptide
MHHHHHHSGSSSGSGSGNALHRVCASNICWRSPWAGRLWVFVRLTSGGSSGSSLGVASAI

SEQ ID NO: 120 - protein, 60 aa, Artificial Sequence
  Description of Artificial Sequence: Synthetic polypeptide
MHQHHHHSGSSSGSGSGITLHWVCASNICWRSPWAGRLWRLVRLTSGGSSGSSLGVASAI

SEQ ID NO: 121 - protein, 60 aa, Artificial Sequence
  Description of Artificial Sequence: Synthetic polypeptide
MHHHHHHSDSSSGSGSGNALHWVCASNICWRSPWAEQLWGLVRLTSGGSSGSSLGVASAI

SEQ ID NO: 122 - protein, 60 aa, Artificial Sequence
  Description of Artificial Sequence: Synthetic polypeptide
MHQHHHHSGSSSGSGSGNALHWVCASNVCWRSPWAGRLWALVRLTSGGSSGSSLGVASAI

SEQ ID NO: 123 - protein, 60 aa, Artificial Sequence
  Description of Artificial Sequence: Synthetic polypeptide
MHHHRHHSGSSSGSGSGNALHWVCASNICWRSPWAGQLWELIRLTSGGSSGSSLGVASAI

SEQ ID NO: 124 - protein, 60 aa, Artificial Sequence
  Description of Artificial Sequence: Synthetic polypeptide
MHHHHHHSGSSSGSGFRNALHWVCASNICWRFPWAGRLWRLVRLTSGGSSGSSLGVASAI

SEQ ID NO: 125 - protein, 60 aa, Artificial Sequence
  Description of Artificial Sequence: Synthetic polypeptide
  MOD_RES (25)..(25): Any amino acid
  MOD_RES (41)..(41): Any amino acid
  MOD_RES (44)..(44): Any amino acid
MHHHHHHSGSSSGSGSGNALRWVCXSNICWRSPWAGRLWGXVRXTSGGSSGSSLGVASAI

SEQ ID NO: 126 - protein, 60 aa, Artificial Sequence
  Description of Artificial Sequence: Synthetic polypeptide
MHHHHHHSGSSSGSGSGNALQWVCASNICWRSPWAGRLRRLVRLTSGGSSGSSLGVASAI

SEQ ID NO: 127 - protein, 60 aa, Artificial Sequence
  Description of Artificial Sequence: Synthetic polypeptide
MHRHHHHSGSSSGSGSGHALHWVCASNICWRSPWARQLWGLVRLTSGGSSGSSLGVASAI

SEQ ID NO: 128 - protein, 60 aa, Artificial Sequence
  Description of Artificial Sequence: Synthetic polypeptide
MHHHHHHSGSSSGCGSGNALHWVCVSNICWRSPWAGRLWGLVRLTSGGSSGSSLGVASAI

SEQ ID NO: 129 - protein, 27 aa, Artificial Sequence
  Description of Artificial Sequence: Synthetic polypeptide
  MOD_RES (1)..(1): Tyr, Asp, Asn, Ile or His
  MOD_RES (4)..(4): Gln or His
  MOD_RES (11)..(11): Val or Ile
  MOD_RES (15)..(15): Thr or Ser
  MOD_RES (18)..(18): Glu or Ala
  MOD_RES (20)..(20): Gln or Arg
  MOD_RES (23)..(23): Arg, Ala, Gly or Glu
  MOD_RES (25)..(25): Ile or Val
XALXWVCASNXCWRXPWXGXLWXLXRL